(19) United States
(12) Patent Application Publication
(10) Pub. No.: US 2018/0256551 A1
(43) Pub. Date: Sep. 13, 2018

(54) METHODS OF TREATING HEMATOLOGICAL MALIGNANCY USING NANOPARTICLE MTOR INHIBITOR COMBINATION THERAPY

(71) Applicant: Abraxis BioScience, LLC, Summit, NJ (US)

(72) Inventors: Neil P. DESAI, Pacific Palisades, CA (US); Mark ALLES, Dallas, PA (US)

(21) Appl. No.: 15/738,087

(22) PCT Filed: Jun. 29, 2016
(86) PCT No.: PCT/US2016/040201

§ 371 Date: Dec. 19, 2017

Related U.S. Application Data
(60) Provisional application No. 62/186,320, filed on Jun. 29, 2015.

(51) Int. Cl. A61K 31/436; A61K 9/51; A61P 35/02

(52) U.S. Cl. CPC A61K 31/436; A61K 9/5169; A61K 45/06; A61K 2300/00; A61P 35/02

(57) ABSTRACT

The present invention relates to methods and compositions for the treatment of hematological malignancy by administering compositions comprising nanoparticles that comprise an mTOR inhibitor (such as a limus drug, e.g., sirolimus or a derivative thereof) and an albumin in combination with compositions comprising a second therapeutic agent.

### CROSS-REFERENCE TO RELATED APPLICATIONS

This application claims priority to U.S. Provisional Application No. 62/186,320, filed on Jun. 29, 2015, which is hereby incorporated by reference in its entirety.

### FIELD OF THE INVENTION

This invention pertains to methods and compositions for the treatment of a hematological malignancy by administering compositions comprising nanoparticles that comprise an mTOR inhibitor (such as a limus drug, e.g., sirolimus or a derivative thereof) and an albumin in combination with a second therapeutic agent.

### BACKGROUND OF THE INVENTION

The mammalian target of rapamycin (mTOR) is a conserved serine/threonine kinase that serves as a central hub of signaling in the cell to integrate intracellular and extracellular signals and to regulate cellular growth and homeostasis. Activation of the mTOR pathway is associated with cell proliferation and survival, while inhibition of mTOR signaling leads to inflammation and cell death. Dysregulation of the mTOR signaling pathway has been implicated in an increasing number of human diseases, including cancer and autoimmune disorders. Consequently, mTOR inhibitors have found wide applications in treating diverse pathological conditions such as solid tumors, hematological malignancies, organ transplantation, restenosis, and rheumatoid arthritis.

Sirolimus (INN/USAN), also known as rapamycin, is an immunosuppressant drug used to prevent rejection in organ transplantation; it is especially useful in kidney transplants. Sirolimus-eluting stents were approved in the United States to treat coronary restenosis. Additionally, sirolimus has been demonstrated as an effective inhibitor of tumor growth in various cell lines and animal models. Other limus drugs, such as analogs of sirolimus, have been designed to improve the pharmacokinetic and pharmacodynamic properties of sirolimus. For example, Temsirolimus was approved in the United States and Europe for the treatment of renal cell carcinoma. Everolimus was approved in the U. S. for treatment of advanced breast cancer, pancreatic neuroendocrine tumors, advanced renal cell carcinoma, and subependymal giant cell astrocytoma (SEGA) associated with Tuberous Sclerosis. The mode of action of sirolimus is to bind the cytosolic protein FK-binding protein 12 (FKBP12), and the sirolimus-FKBP12 complex in turn inhibits the mTOR pathway by directly binding to the mTOR Complex 1 (mTORC1).

Albumin-based nanoparticle compositions have been developed as a drug delivery system for delivering substantially water insoluble drugs. See, for example, U.S. Pat. Nos. 5,916,596; 6,506,405; 6,749,868, and 6,537,579, 7,820,788, and 7,923,536. Abraxane®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as bladder cancer and melanoma. Albumin derived from human blood has been used for the manufacture of Abraxane® as well as various other albumin-based nanoparticle compositions.

The disclosures of all publications, patents, patent applications and published patent applications referred to herein are hereby incorporated herein by reference in their entirety.

### BRIEF SUMMARY OF THE INVENTION

The present invention provides methods of treating a hematological malignancy (such as lymphoma, leukemia, and myeloma) in an individual, comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor nanoparticle composition (such as sirolimus/albumin nanoparticle composition) and the second therapeutic agent act synergistically to inhibit cell proliferation. In some embodiments, the mTOR inhibitor is a limus drug. In some embodiments, the mTOR inhibitor is sirolimus or a derivative thereof. In some embodiments, the mTOR inhibitor is sirolimus. In some embodiments, the albumin is human albumin (such as human serum albumin). In some embodiments, the nanoparticles comprise sirolimus or a derivative thereof associated (e.g., coated) with albumin. In some embodiments, the nanoparticles comprise sirolimus or a derivative thereof coated with albumin. In some embodiments, the average particle size of the nanoparticles in the mTOR inhibitor nanoparticle composition (such as sirolimus/albumin nanoparticle composition) is no greater than about 150 nm (such as no greater than about 120 nm). In some embodiments, the average particle size of the nanoparticles in the mTOR inhibitor nanoparticle composition (such as sirolimus/albumin nanoparticle composition) is no more than about 120 nm. In some embodiments, the nanoparticles in the mTOR inhibitor nanoparticle composition (such as sirolimus/albumin nanoparticle composition) are sterile filterable. In some embodiments, the mTOR inhibitor nanoparticle composition comprises the albumin stabilized nanoparticle formulation of sirolimus (nab-sirolimus, a formulation of sirolimus stabilized by human albumin USP, where the weight ratio of human albumin and sirolimus is about 8:1 to about 9:1). In some embodiments, the mTOR inhibitor nanoparticle composition is nab-sirolimus. In some embodiments, the mTOR inhibitor nanoparticle composition is administered intravenously, intraarterially, intraperitoneally, intravesicularly, subcutaneously, intrathecally, intrapulmonarily, intramuscularly, intratracheally, intraocularly, transdermally, orally, or by inhalation. In some embodiments, the mTOR inhibitor nanoparticle composition is administered intravenously. In some embodiments, the mTOR inhibitor nanoparticle composition is administered subcutaneously. In some embodiments, the individual is a human.

In some embodiments, according to any of the methods described above, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared from a tumor cell or at least one tumor-associated antigen). In some embodiments, the second therapeutic agent is an immunomodulator. In some embodiments, the immunomodulator is an IMiDs® (small molecule immunomodulator, such as lenalidomide and pomalidomide). In some embodiments, the immunomodulator is small molecule or antibody-based IDO inhibitor. In some embodiments, the second therapeutic agent is an immunomodulator that stimulates the immune system (hereinafter also referred to as an “immunostimulator”). In some embodiments, the immunomodulator is an agonistic antibody that targets an activating receptor (including co-stimulatory receptors) on a T cell. In some embodiments, the immunomodulator is an immune checkpoint inhibitor. In some embodiments, the immune checkpoint inhibitor is an antagonistic antibody that targets an immune checkpoint protein. In some embodiments, the second therapeutic agent is an immunomodulator selected from the group consisting of pomalidomide and lenalidomide. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the second therapeutic agent is a kinase inhibitor. In some embodiments, the kinase inhibitor is selected from the group consisting of nilotinib and sorafenib. In some embodiments, the second therapeutic agent is a cancer vaccine. In some embodiments, the cancer vaccine is a vaccine prepared from a tumor cell or a vaccine prepared from at least one tumor-associated antigen.

In some embodiments, according to any of the methods described above, the hematological malignancy is selected from the group consisting of multiple myeloma, mantle cell lymphoma, T cell lymphoma, chronic myeloid leukemia, and acute myeloid leukemia. In some embodiments, the hematological malignancy is a relapsed hematological malignancy. In some embodiments, the hematological malignancy is refractory to a standard therapy for the hematological malignancy. In some embodiments, the second therapeutic agent is an immunomodulator (such as an immunostimulator or an immune checkpoint inhibitor), a histone deacetylase inhibitor, a kinase inhibitor (such as a tyrosine kinase inhibitor), or a cancer vaccine (such as a vaccine prepared using tumor cells or at least one tumor-associated antigen).

In some embodiments, according to any of the methods described above, the hematological malignancy is multiple myeloma, and the second therapeutic agent is pomalidomide. In some embodiments, the hematological malignancy is mantle cell lymphoma, and the second therapeutic agent is lenalidomide. In some embodiments, the hematological malignancy is multiple myeloma, and the second therapeutic agent is romidepsin. In some embodiments, the hematological malignancy is T cell lymphoma, and the second therapeutic agent is romidepsin. In some embodiments, the hematological malignancy is chronic myeloid leukemia, and the second therapeutic agent is nilotinib. In some embodiments, the hematological malignancy is acute myeloid leukemia, and the second therapeutic agent is sorafenib.

In some embodiments, according to any of the methods described above, the mTOR inhibitor nanoparticle composition (such as sirolimus/albumin nanoparticle composition) and the second therapeutic agent are administered simultaneously. In other embodiments, the mTOR inhibitor nanoparticle composition (such as sirolimus/albumin nanoparticle composition) and the second therapeutic agent are not administered simultaneously. In some embodiments, the mTOR inhibitor nanoparticle composition (such as sirolimus/albumin nanoparticle composition) and the second therapeutic agent are administered sequentially.

In some embodiments, according to any of the methods described above, the mTOR inhibitor nanoparticle composition (such as sirolimus/albumin nanoparticle composition) and the second therapeutic agent are present in amounts that produce a synergistic effect in the treatment of a hematological malignancy (such as lymphoma, leukemia, and myeloma) in an individual in need thereof.

In some embodiments, according to any of the methods described above, the method is carried out in a neoadjuvant setting. In some embodiments, the method is carried out in an adjuvant setting.

In some embodiments, according to any of the methods described above, the hematological malignancy is refractory to a standard therapy or recurrent after the standard therapy. In some embodiments, the treatment is first line treatment. In some embodiments, the treatment is second line treatment.

In some embodiments, according to any of the methods described above, the individual has progressed from an earlier therapy for a hematological malignancy. In some embodiments, the individual is refractory to an earlier therapy for a hematological malignancy. In some embodiments, the individual has recurrent hematological malignancy.

In some embodiments, according to any of the methods described above, the amount of the nanoparticles in the mTOR inhibitor nanoparticle composition (such as sirolimus/albumin nanoparticle composition) is about 10 mg/m² to about 200 mg/m² (such as about any of 10, 20, 30, 45, 75, 100, 150, or 200 mg/m², including any range between these values). In some embodiments, the amount of the nanoparticles in the mTOR inhibitor nanoparticle composition (such as sirolimus/albumin nanoparticle composition) is about 45 mg/m². In some embodiments, the amount of the nanoparticles in the mTOR inhibitor nanoparticle composition (such as sirolimus/albumin nanoparticle composition) is about 75 mg/m². In some embodiments, the amount of the nanoparticles in the mTOR inhibitor nanoparticle composition (such as sirolimus/albumin nanoparticle composition) is about 100 mg/m². In some embodiments, the mTOR inhibitor nanoparticle composition (such as sirolimus/albumin nanoparticle composition) is administered weekly (such as 3 out of 4 weeks, e.g., on days 1, 8, and 15 of a 28-day cycle). In some embodiments, the mTOR inhibitor nanoparticle composition (such as sirolimus/albumin nanoparticle composition) is administered at least twice (such as at least 2, 3, or 4 times) in a 28-day cycle for at least one (such at least 2, 3, 4, 5, 6, 7, 8, 9, 10, or more) cycle. In some embodiments, the mTOR inhibitor nanoparticle composition (such as sirolimus/albumin nanoparticle composition) is administered at least twice (such as at least 2, 3, or 4 times) at weekly intervals in a 28-day cycle (such as on days 1, 8, and 15 of the 28-day cycle) for at least one (such at least 2, 3, 4, 5, 6, 7, 8, 9, 10, or more) cycle. In some embodiments, the mTOR inhibitor nanoparticle composition (such as sirolimus/albumin nanoparticle composition) is administered three times in a 28-day cycle (such as on days 1, 8, and 15 of the 28-day cycle) for at least one (such at least 2, 3, 4, 5, 6, 7, 8, 9, 10, or more) cycle.

Also provided are methods of treating a hematological malignancy according to any of the methods described above, wherein the treatment is based on the level of at least one biomarker. In some embodiments, the method further comprises selecting the individual for treatment based on the presence of at least one mTOR-activating aberration. In some embodiments, the mTOR-activating aberration comprises a mutation in an mTOR-associated gene. In some embodiments, the mTOR-activating aberration is in at least one mTOR-associated gene selected from the group consisting of protein kinase B (PKB/Akt), fms-like tyrosine kinase 3 internal tandem duplication (FLT-3ITD), mechanistic target of rapamycin (mTOR), phosphoinositide 3-kinase (PI3K), TSC1, TSC2, RHEB, STK11, NF1, NF2, Kirsten rat sarcoma viral oncogene homolog (KRAS), neuroblastoma RAS viral (v-ras) oncogene homolog (NRAS) and PTEN. In some embodiments, the treatment is based on the presence of at least one genetic variant in a gene selected from the group consisting of drug metabolism genes, cancer genes, and drug target genes.

In some embodiments, according to any of the methods described above, wherein the method comprises administration of an immunomodulator, the method further comprised selecting the individual for treatment based on the presence of at least one biomarker indicative of favorable response to treatment with an immunomodulator. In some embodiments, the at least one biomarker comprises a mutation in an immunomodulator-associated gene.

In some embodiments, according to any of the methods described above, wherein the method comprises administration of a histone deacetylase inhibitor, the method further comprised selecting the individual for treatment based on the presence of at least one biomarker indicative of favorable response to treatment with a histone deacetylase inhibitor (HDACi). In some embodiments, the at least one biomarker comprises a mutation in an HDAC-associated gene.

In some embodiments, according to any of the methods described above, wherein the method comprises administration of a kinase inhibitor, the method further comprised selecting the individual for treatment based on the presence of at least one biomarker indicative of favorable response to treatment with a kinase inhibitor. In some embodiments, the at least one biomarker comprises a mutation in a kinase-associated gene.

In some embodiments, according to any of the methods described above, the method further comprises selecting the individual for treatment based on the presence of at least one biomarker indicative of favorable response to treatment with a cancer vaccine. In some embodiments, the at least one biomarker comprises a tumor-associated antigen (TAA) expressed in tumor cells in the individual, such as an aberrantly expressed protein or a neo-antigen.

The methods described herein can be used for any one or more of the following purposes: alleviating one or more symptoms of a hematological malignancy, delaying progressing of a hematological malignancy, shrinking tumor size in a hematological malignancy patient, inhibiting hematological malignancy tumor growth, prolonging overall survival, prolonging disease-free survival, prolonging time to hematological malignancy progression, preventing or delaying metastasis, reducing (such as eradicating) preexisting metastasis, reducing incidence or burden of preexisting metastasis, and preventing recurrence of hematological malignancy.

### DETAILED DESCRIPTION OF THE INVENTION

The present invention provides methods and compositions for treating a hematological malignancy (such as lymphoma, leukemia, and myeloma) in an individual by administering to the individual a composition comprising nanoparticles comprising an mTOR inhibitor (such as a limus drug, e.g., sirolimus or a derivative thereof) and an albumin (hereinafter also referred to as an “mTOR inhibitor nanoparticle composition”) in conjunction with a second therapeutic agent. The second therapeutic agent may be an immunomodulator (such as an immunostimulator or an immune checkpoint inhibitor), a histone deacetylase inhibitor, a kinase inhibitor (such as a tyrosine kinase inhibitor), or a cancer vaccine (such as a vaccine prepared from a tumor cell or a vaccine prepared from at least one tumor-associated antigen).

The present application thus provides methods of combination therapy. It is to be understood by a person of ordinary skill in the art that the combination therapy methods described herein requires that one agent or composition be administered in conjunction with another agent.

Also provided are compositions (such as pharmaceutical compositions), kits, and unit dosages useful for the methods described herein.

### Definitions

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, —CH═CH(CH₃), —CH(CH₃)₂, —C(CH₃)═H₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclylalkyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

A “halogen” is fluorine, chlorine, bromine or iodine.

A “hydroxyalkyl” group is an alkyl group as described above substituted with one or more hydroxy groups.

An “alkoxy” group is —O-(alkyl), wherein alkyl is defined above.

An “amino” group is a radical of the formula: —NH₂.

A “carboxy” group is a radical of the formula: —C(O)OH.

When the groups described herein, with the exception of alkyl group are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein “nab” stands for nanoparticle albumin-bound, and “nab-sirolimus” is an albumin stabilized nanoparticle formulation of sirolimus. nab-sirolimus is also known as nab-rapamycin, which has been previously described. See, for example, WO2008109163A1, WO2014151853, WO2008137148A2, and WO2012149451A1, each of which is incorporated herein by reference in their entirety.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In some embodiments, the treatment reduces the severity of one or more symptoms associated with cancer by at least about any of 10%, 20%, 30%, 40%, 50%, 60%, 70%, 80%, 90%, 95% or 100% compared to the corresponding symptom in the same subject prior to treatment or compared to the corresponding symptom in other subjects not receiving the treatment. Also encompassed by “treatment” is a reduction of pathological consequence of cancer. The methods of the invention contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer, which are described herein. An individual having one or more of these risk factors has a higher probability of developing cancer than an individual without these risk factor(s).

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT scan), Magnetic Resonance Imaging (MRI), ultrasound, clotting tests, arteriography, biopsy, urine cytology, and cystoscopy. Development may also refer to cancer progression that may be initially undetectable and includes occurrence, recurrence, and onset.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer.

As is understood in the art, an “effective amount” may be in one or more doses, i.e., a single dose or multiple doses may be required to achieve the desired treatment endpoint. An effective amount may be considered in the context of administering one or more therapeutic agents, and a nanoparticle composition (e.g., a composition including sirolimus and an albumin) may be considered to be given in an effective amount if, in conjunction with one or more other agents, a desirable or beneficial result may be or is achieved. The components (e.g., the first and second therapies) in a combination therapy of the invention may be administered sequentially, simultaneously, or concurrently using the same or different routes of administration for each component. Thus, an effective amount of a combination therapy includes an amount of the first therapy and an amount of the second therapy that when administered sequentially, simultaneously, or concurrently produces a desired outcome.

“In conjunction with” or “in combination with” refers to administration of one treatment modality in addition to another treatment modality, such as administration of a nanoparticle composition described herein in addition to administration of the other agent to the same individual under the same treatment plan. As such, “in conjunction with” or “in combination with” refers to administration of one treatment modality before, during or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, “specific”, “specificity”, or “selective” or “selectivity” as used when describing a compound as an inhibitor, means that the compound preferably interacts with (e.g., binds to, modulates, and inhibits) a particular target (e.g., a protein and an enzyme) than a non-target. For example, the compound has a higher affinity, a higher avidity, a higher binding coefficient, or a lower dissociation coefficient for a particular target. The specificity or selectivity of a compound for a particular target can be measured, determined, or assessed by using various methods well known in the art. For example, the specificity or selectivity can be measured, determined, or assessed by measuring the IC₅₀ of a compound for a target. A compound is specific or selective for a target when the IC₅₀ of the compound for the target is 2-fold, 4-fold, 6-fold, 8-fold, 10-fold, 20-fold, 50-fold, 100-fold, 500-fold, 1000-fold, or more lower than the IC₅₀ of the same compound for a non-target. For example, the IC₅₀ of a histone deacetylase inhibitor of the present invention for HDACs is 2-fold, 4-fold, 6-fold, 8-fold, 10-fold, 20-fold, 50-fold, 100-fold, 500-fold, 1000-fold, or more lower than the IC₅₀ of the same histone deacetylase inhibitor for non-HDACs. For example, the IC₅₀ of a histone deacetylase inhibitor of the present invention for class-I HDACs is 2-fold, 4-fold, 6-fold, 8-fold, 10-fold, 20-fold, 50-fold, 100-fold, 500-fold, 1000-fold, or more lower than the IC₅₀ of the same histone deacetylase inhibitor for other HDACs (e.g., class-II HDACs). For example, the IC₅₀ of a histone deacetylase inhibitor of the present invention for HDAC3 is 2-fold, 4-fold, 6-fold, 8-fold, 10-fold, 20-fold, 50-fold, 100-fold, 500-fold, 1000-fold, or more lower than the IC₅₀ of the same histone deacetylase inhibitor for other HDACs (e.g., HDAC1, 2, or 6). IC₅₀ can be determined by commonly known methods in the art.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U. S. Food and Drug administration.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “or,” and “the” include plural referents unless the context clearly dictates otherwise.

### Methods of Treating a Hematological Malignancy

The present invention provides methods of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, and wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), and wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared from a tumor cell or at least one tumor-associated antigen). In some embodiments, the second therapeutic agent is an immunomodulator (such as an immunostimulator or an immune checkpoint inhibitor). In some embodiments, the immunomodulator is an immunostimulator that directly stimulates the immune system. In some embodiments, the immunomodulator is an agonistic antibody that targets an activating receptor on a T cell. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide and pomalidomide). In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. In some embodiments, the hematological malignancy is selected from the group consisting of multiple myeloma, mantle cell lymphoma, T cell lymphoma, chronic myeloid leukemia, and acute myeloid leukemia. In some embodiments, the hematological malignancy is a relapsed or refractory hematological malignancy.

In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent, wherein the nanoparticle composition and the second therapeutic agent are administered concurrently. In some embodiments, the administrations of the nanoparticle composition and the second therapeutic agent are initiated at about the same time (for example, within any one of 1, 2, 3, 4, 5, 6, or 7 days). In some embodiments, the administrations of the nanoparticle composition and the second therapeutic agent are terminated at about the same time (for example, within any one of 1, 2, 3, 4, 5, 6, or 7 days). In some embodiments, the administration of the second therapeutic agent continues (for example for about any one of 0.5, 1, 2, 3, 4, 5, 6, 7, 8, 9, 10, 11, or 12 months) after the termination of the administration of the nanoparticle composition. In some embodiments, the administration of the second therapeutic agent is initiated after (for example after about any one of 0.5, 1, 2, 3, 4, 5, 6, 7, 8, 9, 10, 11, or 12 months) the initiation of the administration of the nanoparticle composition. In some embodiments, the administrations of the nanoparticle composition and the second therapeutic agent are initiated and terminated at about the same time. In some embodiments, the administrations of the nanoparticle composition and the second therapeutic agent are initiated at about the same time and the administration of the second therapeutic agent continues (for example for about any one of 0.5, 1, 2, 3, 4, 5, 6, 7, 8, 9, 10, 11, or 12 months) after the termination of the administration of the nanoparticle composition. In some embodiments, the administration of the nanoparticle composition and the second therapeutic agent stop at about the same time and the administration of the second therapeutic agent is initiated after (for example after about any one of 0.5, 1, 2, 3, 4, 5, 6, 7, 8, 9, 10, 11, or 12 months) the initiation of the administration of the nanoparticle composition. In some embodiments, the administration of the nanoparticle composition and the second therapeutic agent stop at about the same time and the administration of the nanoparticle composition is initiated after (for example after about any one of 0.5, 1, 2, 3, 4, 5, 6, 7, 8, 9, 10, 11, or 12 months) the initiation of the administration of the second therapeutic agent.

“mTOR inhibitor” used herein refers to inhibitors of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression. In some embodiments, the mTOR inhibitor is an mTOR kinase inhibitor. mTOR inhibitors described herein include, but are not limited to, BEZ235 (NVP-BEZ235), everolimus (also known as RAD001, Zortress, Certican, and Afinitor), rapamycin (also known as sirolimus or Rapamune), AZD8055,temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

In some embodiments, the mTOR inhibitor is a limus drug, which includes sirolimus and its analogues. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

Thus, for example, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin, wherein the mTOR inhibitor is selected from the group consisting of BEZ235 (NVP-BEZ235), everolimus (also known as RAD001, Zortress, Certican, and Afinitor), rapamycin (also known as sirolimus or Rapamune), AZD8055,temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus); and b) an effective amount of a second therapeutic agent.

In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin, wherein the mTOR inhibitor is a limus drug selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506); and b) an effective amount of a second therapeutic agent.

In some embodiments, the second therapeutic agent is an immunomodulator. In some embodiments, the immunomodulator is an immunostimulator. In some embodiments, the immunostimulator directly stimulates the immune system. In some embodiments, the immunomodulator is an IMiDs® (Celgene). IMiDs® compounds are proprietary small molecule, orally available compounds that modulate the immune system and other biological targets through multiple mechanisms of action. In some embodiments, the immunomodulator is small molecule or antibody-based IDO inhibitor. In some embodiments, the immunomodulator is selected from the group consisting of a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, and pomalidomide. In some embodiments, the immunomodulator is lenalidomide. In some embodiments, the immunomodulator is pomalidomide. In some embodiments, the immunomodulator is an agonistic antibody that targets an activating receptor (including co-stimulatory receptors) on a T cell. In some embodiments, the immunomodulator is an agonistic antibody selected from the group consisting of anti-CD28, anti-OX40 (such as MEDI6469), anti-ICOS (such as JTX-2011, Jounce Therapeutics), anti-GITR (such as TRX518), anti-4-1BB (such as BMS-663513 and PF-05082566), anti-CD27 (such as Varlilumab and hCD27.15), anti-CD40 (such as CP870,893), and anti-HVEM. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MEDI4736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-MR (such as Lirilumab and IPH2101), anti-A2aR, anti-CD52 (such as alemtuzumab), anti-IL-10, anti-IL-35, anti-FasL, and anti-TGF-β (such as Fresolumimab).

Thus, for example, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator. In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunostimulator. In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunostimulator that directly stimulates the immune system of the individual. In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an IMiDs® (small molecule immunomodulator, such as lenalidomide and pomalidomide). In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a small molecule or antibody-based IDO inhibitor.

In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as an immunostimulator) selected from the group consisting of a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, and pomalidomide. In some embodiments, the immunomodulator is lenalidomide. In some embodiments, the immunomodulator is pomalidomide.

In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an agonist of an activating receptor (including co-stimulatory receptors) on a T cell. In some embodiments, the agonist of an activating receptor (including co-stimulatory receptors) on a T cell is an agonistic antibody selected from the group consisting of anti-CD28, anti-OX40 (such as MEDI6469), anti-ICOS (such as JTX-2011, Jounce Therapeutics), anti-GITR (such as TRX518), anti-4-1BB (such as BMS-663513 and PF-05082566), anti-CD27 (such as Varlilumab and hCD27.15), anti-CD40 (such as CP870,893), and anti-HVEM.

In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immune checkpoint inhibitor. In some embodiments, the immune checkpoint inhibitor is an antagonistic antibody that targets an immune checkpoint protein. In some embodiments, the immune checkpoint inhibitor is an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MEDI4736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-IL-35, anti-FasL, and anti-TGF-β (such as Fresolumimab).

In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of vorinostat (SAHA), panobinostat (LBH589), belinostat (PXD101, CAS 414864-00-9), tacedinaline (N-acetyldinaline, CI-994), givinostat (gavinostat, ITF2357), FRM-0334 (EVP-0334), resveratrol (SRT501), CUDC-101, quisinostat (JNJ-26481585), abexinostat (PCI-24781), dacinostat (LAQ824, NVP-LAQ824), valproic acid, 4-(dimethylamino) N-[6-(hydroxyamino)-6-oxohexyl]-benzamide (HDAC1 inhibitor), 4-Iodo suberoylanilide hydroxamic acid (HDAC1 and HDAC6 inhibitor), romidepsin (a cyclic tetrapeptide with HDAC inhibitory activity primarily towards class-I HDACs), 1-naphthohydroxamic acid (HDAC1 and HDAC6 inhibitor), HDAC inhibitors based on amino-benzamide biasing elements (e.g., mocetinostat (MGCD103) and entinostat (MS275), which are highly selective for HDAC1, 2 and 3), AN-9 (CAS 122110-53-6), APHA Compound 8 (CAS 676599-90-9), apicidin (CAS 183506-66-3), BML-210 (CAS 537034-17-6), salermide (CAS 1105698-15-4), suberoyl bis-hydroxamic acid (CAS 38937-66-5) (HDAC1 and HDAC3 inhibitor), butyrylhydroxamic acid (CAS 4312-91-8), CAY10603 (CAS 1045792-66-2) (HDAC6 inhibitor), CBHA (CAS 174664-65-4), ricolinostat (ACY1215, rocilinostat), trichostatin-A, WT-161, tubacin, and Merck60. In some embodiments, the second therapeutic agent is the histone deacetylase inhibitor romidepsin.

Thus, for example, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is a hydroxamic acid, including, but not limited to, vorinostat (suberoylanilide hydroxamic acid or “SAHA”), trichostatin A (“TSA”), LBH589 (panobinostat), PXD101 (belinostat), oxamflatin, tubacin, seriptaid, NVP-LAQ824, cinnamic acid hydroxamic acid (CBHA), CBHA derivatives, and ITF2357. In some embodiments, the histone deacetylase inhibitor is a benzamide, including, but not limited to, mocetinostat (MGCD0103), benzamide M344, BML-210, entinostat (SNDX-275 or MS-275), pimelic diphenylamide 4b, pimelic diphenylamide 106, MS-994, CI-994 (acetyldinaline, PD 123654, and 4-acetylamino-N-(Uaminophenyl)-benzamide). In some embodiments, the histone deacetylase inhibitor is romidepsin.

In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib. In some embodiments, the second therapeutic agent is the kinase inhibitor nilotinib. In some embodiments, the second therapeutic agent is the kinase inhibitor sorafenib.

Thus, for example, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib. In some embodiments, the kinase inhibitor is nilotinib. In some embodiments, the kinase inhibitor is sorafenib.

In some embodiments, the second therapeutic agent is a cancer vaccine, such as a vaccine prepared using autologous or allogeneic tumor cells. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using at least one tumor-associated antigen (TAA).

Thus, for example, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using at least one tumor-associated antigen (TAA).

Reference to a second therapeutic agent herein applies to the second therapeutic agent or its derivatives and accordingly the invention contemplates and includes either of these embodiments (second therapeutic agent; second therapeutic agent or derivative(s) thereof). “Derivatives” or “analogs” of an agent or other chemical moiety include, but are not limited to, compounds that are structurally similar to the agent or moiety or are in the same general chemical class as the agent or moiety. In some embodiments, the derivative or analog of the second therapeutic agent or moiety retains similar chemical and/or physical property (including, for example, functionality) of the second therapeutic agent or moiety.

In some embodiments, according to any of the methods described herein, the method further comprises administering to the individual one or more additional therapeutic agents used in a standard combination therapy with the second therapeutic agent. Thus, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; b) an effective amount of a second therapeutic agent; and c) an effective amount of at least one therapeutic agent used in a standard combination therapy with the second therapeutic agent.

The methods provided herein can be used to treat an individual (e.g., human) who has been diagnosed with or is suspected of having a hematological malignancy. In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as multiple myeloma, mantle cell lymphoma, T cell lymphoma, chronic myeloid leukemia, and acute myeloid leukemia). In some embodiments, the individual has one or more risk factors associated with one or more diseases or disorders described herein.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In some embodiments, the efficacy of treatment is measured as the percentage tumor growth inhibition (% TGI), calculated using the equation 100−(T/C×100), where T is the mean relative tumor volume of the treated tumor, and C is the mean relative tumor volume of a non-treated tumor. In some embodiments, the % TGI is about 10%, about 20%, about 30%, about 40%, about 50%, about 60%, about 70%, about 80%, about 90%, about 91%, about 92%, about 93%, about 94%, about 95%, or more than 95%.

### Plasmacytoma

In some embodiments, there is provided a method of treating plasmacytoma (such as multiple myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor) and a histone deacetylase inhibitor. In some embodiments, the second therapeutic agent is an immunomodulator. In some embodiments, the immunomodulator is an immunostimulator that directly stimulates the immune system. In some embodiments, the immunomodulator is an agonistic antibody that targets an activating receptor on a T cell. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide and pomalidomide). In some embodiments, the immunomodulator is small molecule or antibody-based IDO inhibitor. In some embodiments, the immunomodulator is pomalidomide. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent is an anti-CD38 antibody (such as daratumumab). In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. Plasmacytoma includes, but is not limited to, myeloma. Myeloma includes, but is not limited to, an extramedullary plasmacytoma, a solitary myeloma, and multiple myeloma. In some embodiments, the plasmacytoma is multiple myeloma. In some embodiments, the multiple myeloma is relapsed or refractory to standard therapy.

In some embodiments, there is provided a method of treating multiple myeloma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent is an anti-CD38 antibody (such as daratumumab). In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as an immunostimulator, e.g., pomalidomide). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of pomalidomide. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of pomalidomide. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of pomalidomide. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of pomalidomide. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of pomalidomide. In some embodiments, the method further comprises administering to the individual at least one therapeutic agent used in a standard combination therapy with pomalidomide, such as dexamethasone. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of romidepsin. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of romidepsin. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of romidepsin. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of romidepsin. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of romidepsin. In some embodiments, the method further comprises administering to the individual at least one therapeutic agent used in a standard combination therapy with romidepsin. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an anti-CD38 antibody. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an anti-CD38 antibody. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an anti-CD38 antibody. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an anti-CD38 antibody. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an anti-CD38 antibody. In some embodiments, the method further comprises administering to the individual at least one therapeutic agent used in a standard combination therapy with an anti-CD38 antibody. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the anti-CD38 antibody is daratumumab. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system. In some embodiments, the immunomodulator is an agonistic antibody that targets an activating receptor on a T cell. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide and pomalidomide). In some embodiments, the immunomodulator is small molecule or antibody-based IDO inhibitor. In some embodiments, the immunomodulator is pomalidomide. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising sirolimus or a derivative thereof and an albumin; and b) an effective amount of pomalidomide. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of pomalidomide. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of pomalidomide. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of pomalidomide. In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of pomalidomide. In some embodiments, the method further comprises administering to the individual at least one therapeutic agent used in a standard combination therapy with pomalidomide, such as dexamethasone. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising sirolimus or a derivative thereof and an albumin, wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 1 to about 4 mg/day (including for example about any of 1, 1.5, 2, 2.5, 3, 3.5, or 4 mg/day) pomalidomide. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 1 to about 4 mg/day (including for example about any of 1, 1.5, 2, 2.5, 3, 3.5, or 4 mg/day) pomalidomide. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 1 to about 4 mg/day (including for example about any of 1, 1.5, 2, 2.5, 3, 3.5, or 4 mg/day) pomalidomide. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 1 to about 4 mg/day (including for example about any of 1, 1.5, 2, 2.5, 3, 3.5, or 4 mg/day) pomalidomide. In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 1 to about 4 mg/day (including for example about any of 1, 1.5, 2, 2.5, 3, 3.5, or 4 mg/day) pomalidomide. In some embodiments, the method further comprises administering to the individual at least one therapeutic agent used in a standard combination therapy with pomalidomide, such as, but not limited to, about 20 to about 40 (including for example about any of 20, 25, 30, 35, 40, and any ranges between these values) mg/week dexamethasone. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the pomalidomide is administered orally. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising sirolimus or a derivative thereof and an albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 4 mg/day pomalidomide; and c) about 40 mg/week dexamethasone. In some embodiments, the method comprises administering to the individual a) an effective amount of a composition comprising nanoparticles comprising sirolimus or a derivative thereof and an albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 4 mg/day pomalidomide; and c) about 40 mg/week dexamethasone. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 4 mg/day pomalidomide; and c) about 40 mg/week dexamethasone. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 4 mg/day pomalidomide; and c) about 40 mg/week dexamethasone. In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 4 mg/day pomalidomide; and c) about 40 mg/week dexamethasone. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the pomalidomide is administered orally. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising sirolimus or a derivative thereof and an albumin; and b) an effective amount of romidepsin. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of romidepsin. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of romidepsin. In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of romidepsin. In some embodiments, the method further comprises administering to the individual at least one therapeutic agent used in a standard combination therapy with romidepsin. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising sirolimus or a derivative thereof and an albumin, wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method further comprises administering to the individual at least one therapeutic agent used in a standard combination therapy with romidepsin. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the romidepsin is administered intravenously. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, there is provided a method of treating multiple myeloma in an individual (such as a human) comprising administering to the individual a) an effective amount of a composition comprising nanoparticles comprising sirolimus or a derivative thereof and an albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method further comprises administering to the individual at least one therapeutic agent used in a standard combination therapy with romidepsin. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the romidepsin is administered intravenously. In some embodiments, the multiple myeloma is recurrent multiple myeloma. In some embodiments, the multiple myeloma is refractory to one or more drugs used in a standard therapy for multiple myeloma, such as, but not limited to, bortezomib, dexamethasone (Dex), doxorubicin (Dox), and melphalan. In some embodiments, the multiple myeloma is selected from the group consisting of IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments, according to any of the methods of treating multiple myeloma in an individual described herein, the individual is a human who exhibits one or more symptoms associated with multiple myeloma. In some embodiments, the individual is at an early stage of multiple myeloma. In some embodiments, the individual is at an advanced stage of multiple myeloma. In some of embodiments, the individual is genetically or otherwise predisposed (e.g., having a risk factor) to developing multiple myeloma. Individuals at risk for multiple myeloma include, e.g., those having relatives who have experienced multiple myeloma, and those whose risk is determined by analysis of genetic or biochemical markers. In some embodiments, the individual may be a human who has a gene, genetic mutation, or polymorphism associated with multiple myeloma (e.g., ras, PTEN, RbI, MTS1/p16INK4A/CDKN2, MTS2/p15INK4B, and/or p53) or has one or more extra copies of a gene associated with multiple myeloma. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

Lymphoid Neoplasm

In some embodiments, there is provided a method of treating a lymphoid neoplasm in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared from a tumor cell or at least one tumor-associated antigen). In some embodiments, the second therapeutic agent is an immunomodulator. In some embodiments, the immunomodulator is an immunostimulator that directly stimulates the immune system. In some embodiments, the immunomodulator is an agonistic antibody that targets an activating receptor on a T cell. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide and pomalidomide). In some embodiments, the immunomodulator is lenalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. In some embodiments the lymphoid neoplasm (e.g., lymphoma or leukemia) is a B-cell neoplasm. In some embodiments, the lymphoid neoplasm (e.g., lymphoma or leukemia) is a T-cell and/or putative NK-cell neoplasm.

In some embodiments, according to any one of the methods of treating a lymphoid neoplasm in an individual described herein, the lymphoid neoplasm (e.g., lymphoma or leukemia) is a B-cell neoplasm. Examples of B-cell neoplasms include, but are not limited to, precursor B-cell neoplasms (e.g., precursor B-lymphoblastic leukemia/lymphoma) and peripheral B-cell neoplasms (e.g., B-cell chronic lymphocytic leukemia/prolymphocytic leukemia/small lymphocytic lymphoma (small lymphocytic (SL) NHL), lymphoplasmacytoid lymphoma/immunocytoma, mantel cell lymphoma, follicle center lymphoma, follicular lymphoma (cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (extranodal (MALT-type+/−monocytoid B cells) and/or Nodal (+/−monocytoid B cells)), splenic marginal zone lymphoma (+/−villous lymphocytes), Hairy cell leukemia, plasmacytoma/plasma cell myeloma (e.g., myeloma and multiple myeloma), diffuse large B-cell lymphoma (primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, and Waldenstrom's macroglobulinemia). In some embodiments, the lymphoid neoplasm is relapsed or refractory to standard therapy.

In some embodiments, according to any one of the methods of treating a lymphoid neoplasm in an individual described herein, the lymphoid neoplasm (e.g., lymphoma or leukemia) is a T-cell and/or putative NK-cell neoplasm. Examples of T-cell and/or putative NK-cell neoplasms include, but are not limited to, precursor T-cell neoplasm (precursor T-lymphoblastic lymphoma/leukemia) and peripheral T-cell and NK-cell neoplasms (T-cell chronic lymphocytic leukemia/prolymphocytic leukemia, large granular lymphocyte leukemia (LGL) (T-cell type and/or NK-cell type), cutaneous T-cell lymphoma (mycosis fungoides/Sezary syndrome), primary T-cell lymphomas unspecified (cytological categories: medium-sized cell, mixed medium and large cell, large cell, and lymphoepitheloid cell and/or subtype hepatosplenic γδ T-cell lymphoma, subcutaneous panniculitic T-cell lymphoma), angioimmunoblastic T-cell lymphoma (AILD), angiocentric lymphoma, intestinal T-cell lymphoma (+/−enteropathy associated), adult T-cell lymphoma/leukemia (ATL), anaplastic large cell lymphoma (ALCL) (CD30+, T- and null-cell types), anaplastic large-cell lymphoma, and Hodgkin's like).

In some embodiments, according to any one of the methods of treating a lymphoid neoplasm in an individual described herein, the lymphoid neoplasm (e.g., lymphoma or leukemia) is Hodgkin's disease. For example, the Hodgkin's disease may be lymphocyte predominance, nodular sclerosis, mixed cellularity, lymphocyte depletion, and/or lymphocyte-rich.

In some embodiments, according to any one of the methods of treating a lymphoid neoplasm in an individual described herein, the lymphoid neoplasm is leukemia, such as chronic leukemia. Examples of chronic leukemia include, but are not limited to, chronic myelocytic I (granulocytic) leukemia, chronic myeloid leukemia (CML), and chronic lymphocytic leukemia. In some embodiments, the leukemia is acute leukemia. Examples of acute leukemia include, but are not limited to, acute lymphoblastic leukemia, acute myeloid leukemia (AML), acute lymphocytic leukemia, and acute myelocytic leukemia (e.g., myeloblastic, promyelocytic, myelomonocytic, monocytic, and erythroleukemia).

### Mantle Cell Lymphoma

Thus, in some embodiments, there is provided a method of treating mantle cell lymphoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. In some embodiments, the mantle cell lymphoma is relapsed or refractory to standard therapy.

In some embodiments, there is provided a method of treating mantle cell lymphoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as an immunostimulator, e.g., lenalidomide). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as an immunostimulator, e.g., lenalidomide). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as an immunostimulator, e.g., lenalidomide). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as an immunostimulator, e.g., lenalidomide). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as an immunostimulator, e.g., lenalidomide). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system. In some embodiments, the immunomodulator is an agonistic antibody that targets an activating receptor on a T cell. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide and pomalidomide). In some embodiments, the immunomodulator is lenalidomide. In some embodiments, the immunomodulator is small molecule or antibody-based IDO inhibitor. In some embodiments, the mantle cell lymphoma is recurrent mantle cell lymphoma. In some embodiments, the mantle cell lymphoma is refractory to one or more drugs used in a standard therapy for mantle cell lymphoma, such as, but not limited to, rituximab, cyclophosphamide, doxorubicin, vincristine, prednisone, bortezomib, cytarabine, methotrexate, bendamustine, fludarabine, mitoxantrone, dexamethasone, and cisplatin.

In some embodiments, there is provided a method of treating mantle cell lymphoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of lenalidomide. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of lenalidomide. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of lenalidomide. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of lenalidomide. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of lenalidomide. In some embodiments, the method further comprises administering to the individual at least one therapeutic agent used in a standard combination therapy with lenalidomide. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the mantle cell lymphoma is recurrent mantle cell lymphoma. In some embodiments, the mantle cell lymphoma is refractory to one or more drugs used in a standard therapy for mantle cell lymphoma, such as, but not limited to, rituximab, cyclophosphamide, doxorubicin, vincristine, prednisone, bortezomib, cytarabine, methotrexate, bendamustine, fludarabine, mitoxantrone, dexamethasone, and cisplatin.

In some embodiments, there is provided a method of treating mantle cell lymphoma in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., lenalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., lenalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., lenalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., lenalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of an immunomodulator (such as an immunostimulator, e.g., lenalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system. In some embodiments, the immunomodulator is an agonistic antibody that targets an activating receptor on a T cell. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide and pomalidomide). In some embodiments, the immunomodulator is lenalidomide. In some embodiments, the immunomodulator is small molecule or antibody-based IDO inhibitor. In some embodiments, the mantle cell lymphoma is recurrent mantle cell lymphoma. In some embodiments, the mantle cell lymphoma is refractory to one or more drugs used in a standard therapy for mantle cell lymphoma, such as, but not limited to, rituximab, cyclophosphamide, doxorubicin, vincristine, prednisone, bortezomib, cytarabine, methotrexate, bendamustine, fludarabine, mitoxantrone, dexamethasone, and cisplatin.

In some embodiments, there is provided a method of treating mantle cell lymphoma in an individual (such as a human) comprising administering to the individual a) an effective amount of a composition comprising nanoparticles comprising sirolimus or a derivative thereof and an albumin; and b) an effective amount of lenalidomide. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of lenalidomide. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of lenalidomide. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of lenalidomide. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of lenalidomide. In some embodiments, the method further comprises administering to the individual at least one therapeutic agent used in a standard combination therapy with lenalidomide. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the mantle cell lymphoma is recurrent mantle cell lymphoma. In some embodiments, the mantle cell lymphoma is refractory to one or more drugs used in a standard therapy for mantle cell lymphoma, such as, but not limited to, rituximab, cyclophosphamide, doxorubicin, vincristine, prednisone, bortezomib, cytarabine, methotrexate, bendamustine, fludarabine, mitoxantrone, dexamethasone, and cisplatin.

In some embodiments, there is provided a method of treating mantle cell lymphoma in an individual (such as a human) comprising administering to the individual a) an effective amount of a composition comprising nanoparticles comprising sirolimus or a derivative thereof and an albumin, wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 15 to about 25 mg/day (including for example about any of 15, 16, 17, 18, 19, 20, 21, 22, 23, 24, or 25 mg/day) lenalidomide. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 15 to about 25 mg/day (including for example about any of 15, 16, 17, 18, 19, 20, 21, 22, 23, 24, or 25 mg/day) lenalidomide. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 15 to about 25 mg/day (including for example about any of 15, 16, 17, 18, 19, 20, 21, 22, 23, 24, or 25 mg/day) lenalidomide. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 15 to about 25 mg/day (including for example about any of 15, 16, 17, 18, 19, 20, 21, 22, 23, 24, or 25 mg/day) lenalidomide. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 15 to about 25 mg/day (including for example about any of 15, 16, 17, 18, 19, 20, 21, 22, 23, 24, or 25 mg/day) lenalidomide. In some embodiments, the method further comprises administering to the individual at least one therapeutic agent used in a standard combination therapy with lenalidomide. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the lenalidomide is administered orally. In some embodiments, the mantle cell lymphoma is recurrent mantle cell lymphoma. In some embodiments, the mantle cell lymphoma is refractory to one or more drugs used in a standard therapy for mantle cell lymphoma, such as, but not limited to, rituximab, cyclophosphamide, doxorubicin, vincristine, prednisone, bortezomib, cytarabine, methotrexate, bendamustine, fludarabine, mitoxantrone, dexamethasone, and cisplatin.

In some embodiments, according to any of the methods of treating mantle cell lymphoma in an individual described herein, the individual is a human who exhibits one or more symptoms associated with mantle cell lymphoma. In some embodiments, the individual is at an early stage of mantle cell lymphoma. In some embodiments, the individual is at an advanced stage of mantle cell lymphoma. In some of embodiments, the individual is genetically or otherwise predisposed (e.g., having a risk factor) to developing mantle cell lymphoma. Individuals at risk for mantle cell lymphoma include, e.g., those having relatives who have experienced mantle cell lymphoma, and those whose risk is determined by analysis of genetic or biochemical markers. In some embodiments, the individual may be a human who has a gene, genetic mutation, or polymorphism associated with mantle cell lymphoma (e.g., cyclin D1, cyclin D2, cyclin D3, β-2 microglobulin, t(11;14)) or has one or more extra copies of a gene associated with mantle cell lymphoma. In some embodiments, the individual has chromosomal translocation t(11;14) (such as t(11;14)(q13;q32)). In some embodiments, the cancer cells have increased expression of cyclin D1 compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

In some embodiments, there is provided a method of treating mantle cell lymphoma in an individual (such as a human) comprising administering to the individual a) an effective amount of a composition comprising nanoparticles comprising sirolimus or a derivative thereof and an albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 25 mg/day lenalidomide; and c) about 40 mg/week dexamethasone. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 25 mg/day lenalidomide; and c) about 40 mg/week dexamethasone. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 25 mg/day lenalidomide; and c) about 40 mg/week dexamethasone. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 25 mg/day lenalidomide; and c) about 40 mg/week dexamethasone. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; b) about 25 mg/day lenalidomide; and c) about 40 mg/week dexamethasone. In some embodiments, the method further comprises administering to the individual at least one therapeutic agent used in a standard combination therapy with lenalidomide. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the lenalidomide is administered orally. In some embodiments, the mantle cell lymphoma is recurrent mantle cell lymphoma. In some embodiments, the mantle cell lymphoma is refractory to one or more drugs used in a standard therapy for mantle cell lymphoma, such as, but not limited to, rituximab, cyclophosphamide, doxorubicin, vincristine, prednisone, bortezomib, cytarabine, methotrexate, bendamustine, fludarabine, mitoxantrone, dexamethasone, and cisplatin.

### T Cell Lymphoma

In some embodiments, there is provided a method of treating T cell lymphoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. T cell lymphoma includes, but is not limited to, cutaneous T cell lymphoma (such as mycosis fungoides and Sezary syndrome), angioimmunoblastic T cell lymphoma, extranodal NK/T cell lymphoma, nasal type, enteropathy-associated intestinal T cell lymphoma (EATL), and anaplastic large cell lymphoma (ALCL). In some embodiments, the T cell lymphoma is cutaneous T cell lymphoma. In some embodiments, the T cell lymphoma is angioimmunoblastic T cell lymphoma. In some embodiments, the T cell lymphoma is extranodal NK/T cell lymphoma, nasal type. In some embodiments, the T cell lymphoma is enteropathy-associated intestinal T cell lymphoma. In some embodiments, the T cell lymphoma is anaplastic large cell lymphoma. In some embodiments, the T cell lymphoma is relapsed or refractory to standard therapy.

In some embodiments, there is provided a method of treating T cell lymphoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the T cell lymphoma is recurrent T cell lymphoma. In some embodiments, the T cell lymphoma is refractory to one or more drugs used in a standard therapy for T cell lymphoma, such as, but not limited to, interferon, zidovudine, cyclophosphamide, doxorubicin, vincristine, prednisone, cisplatin, etoposide, ifosfamide, carboplatin, dexamethasone, methotrexate, brentuximab vedotin, pralatrexate, bortezomib, belinostat, alemtuzumab, denileukin diftitox, and romidepsin. In some embodiments, the T cell lymphoma is selected from the group consisting of cutaneous T cell lymphoma (such as mycosis fungoides and Sezary syndrome), angioimmunoblastic T cell lymphoma, extranodal NK/T cell lymphoma, nasal type, enteropathy-associated intestinal T cell lymphoma (EATL), and anaplastic large cell lymphoma (ALCL). In some embodiments, the T cell lymphoma is cutaneous T cell lymphoma. In some embodiments, the T cell lymphoma is angioimmunoblastic T cell lymphoma. In some embodiments, the T cell lymphoma is extranodal NK/T cell lymphoma, nasal type. In some embodiments, the T cell lymphoma is enteropathy-associated intestinal T cell lymphoma. In some embodiments, the T cell lymphoma is anaplastic large cell lymphoma.

In some embodiments, there is provided a method of treating T cell lymphoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of romidepsin. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of romidepsin. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of romidepsin. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of romidepsin. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of romidepsin. In some embodiments, the method further comprises administering to the individual at least one therapeutic agent used in a standard combination therapy with romidepsin. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the T cell lymphoma is recurrent T cell lymphoma. In some embodiments, the T cell lymphoma is refractory to one or more drugs used in a standard therapy for T cell lymphoma, such as, but not limited to, interferon, zidovudine, cyclophosphamide, doxorubicin, vincristine, prednisone, cisplatin, etoposide, ifosfamide, carboplatin, dexamethasone, methotrexate, brentuximab vedotin, pralatrexate, bortezomib, belinostat, alemtuzumab, denileukin diftitox, and romidepsin. In some embodiments, the T cell lymphoma is selected from the group consisting of cutaneous T cell lymphoma (such as mycosis fungoides and Sezary syndrome), angioimmunoblastic T cell lymphoma, extranodal NK/T cell lymphoma, nasal type, enteropathy-associated intestinal T cell lymphoma (EATL), and anaplastic large cell lymphoma (ALCL). In some embodiments, the T cell lymphoma is cutaneous T cell lymphoma. In some embodiments, the T cell lymphoma is angioimmunoblastic T cell lymphoma. In some embodiments, the T cell lymphoma is extranodal NK/T cell lymphoma, nasal type. In some embodiments, the T cell lymphoma is enteropathy-associated intestinal T cell lymphoma. In some embodiments, the T cell lymphoma is anaplastic large cell lymphoma.

In some embodiments, there is provided a method of treating T cell lymphoma in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the T cell lymphoma is recurrent T cell lymphoma. In some embodiments, the T cell lymphoma is refractory to one or more drugs used in a standard therapy for T cell lymphoma, such as, but not limited to, interferon, zidovudine, cyclophosphamide, doxorubicin, vincristine, prednisone, cisplatin, etoposide, ifosfamide, carboplatin, dexamethasone, methotrexate, brentuximab vedotin, pralatrexate, bortezomib, belinostat, alemtuzumab, denileukin diftitox, and romidepsin. In some embodiments, the T cell lymphoma is selected from the group consisting of cutaneous T cell lymphoma (such as mycosis fungoides and Sezary syndrome), angioimmunoblastic T cell lymphoma, extranodal NK/T cell lymphoma, nasal type, enteropathy-associated intestinal T cell lymphoma (EATL), and anaplastic large cell lymphoma (ALCL). In some embodiments, the T cell lymphoma is cutaneous T cell lymphoma. In some embodiments, the T cell lymphoma is angioimmunoblastic T cell lymphoma. In some embodiments, the T cell lymphoma is extranodal NK/T cell lymphoma, nasal type. In some embodiments, the T cell lymphoma is enteropathy-associated intestinal T cell lymphoma. In some embodiments, the T cell lymphoma is anaplastic large cell lymphoma.

In some embodiments, there is provided a method of treating T cell lymphoma in an individual (such as a human) comprising administering to the individual a) an effective amount of a composition comprising nanoparticles comprising sirolimus or a derivative thereof and an albumin; and b) an effective amount of romidepsin. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of romidepsin. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of romidepsin. In some embodiments, the method further comprises administering to the individual at least one therapeutic agent used in a standard combination therapy with romidepsin. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the T cell lymphoma is recurrent T cell lymphoma. In some embodiments, the T cell lymphoma is refractory to one or more drugs used in a standard therapy for T cell lymphoma, such as, but not limited to, interferon, zidovudine, cyclophosphamide, doxorubicin, vincristine, prednisone, cisplatin, etoposide, ifosfamide, carboplatin, dexamethasone, methotrexate, brentuximab vedotin, pralatrexate, bortezomib, belinostat, alemtuzumab, denileukin diftitox, and romidepsin. In some embodiments, the T cell lymphoma is selected from the group consisting of cutaneous T cell lymphoma (such as mycosis fungoides and Sezary syndrome), angioimmunoblastic T cell lymphoma, extranodal NK/T cell lymphoma, nasal type, enteropathy-associated intestinal T cell lymphoma (EATL), and anaplastic large cell lymphoma (ALCL). In some embodiments, the T cell lymphoma is cutaneous T cell lymphoma. In some embodiments, the T cell lymphoma is angioimmunoblastic T cell lymphoma. In some embodiments, the T cell lymphoma is extranodal NK/T cell lymphoma, nasal type. In some embodiments, the T cell lymphoma is enteropathy-associated intestinal T cell lymphoma. In some embodiments, the T cell lymphoma is anaplastic large cell lymphoma.

In some embodiments, there is provided a method of treating T cell lymphoma in an individual (such as a human) comprising administering to the individual a) an effective amount of a composition comprising nanoparticles comprising sirolimus or a derivative thereof and an albumin, wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 5 to about 14 mg/m² (including for example about any of 5, 6, 7, 8, 9, 10, 11, 12, 13, or 14 mg/m²) romidepsin. In some embodiments, the method further comprises administering to the individual at least one therapeutic agent used in a standard combination therapy with romidepsin. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the romidepsin is administered intravenously. In some embodiments, the T cell lymphoma is recurrent T cell lymphoma. In some embodiments, the T cell lymphoma is refractory to one or more drugs used in a standard therapy for T cell lymphoma, such as, but not limited to, interferon, zidovudine, cyclophosphamide, doxorubicin, vincristine, prednisone, cisplatin, etoposide, ifosfamide, carboplatin, dexamethasone, methotrexate, brentuximab vedotin, pralatrexate, bortezomib, belinostat, alemtuzumab, denileukin diftitox, and romidepsin. In some embodiments, the T cell lymphoma is selected from the group consisting of cutaneous T cell lymphoma (such as mycosis fungoides and Sezary syndrome), angioimmunoblastic T cell lymphoma, extranodal NK/T cell lymphoma, nasal type, enteropathy-associated intestinal T cell lymphoma (EATL), and anaplastic large cell lymphoma (ALCL). In some embodiments, the T cell lymphoma is cutaneous T cell lymphoma. In some embodiments, the T cell lymphoma is angioimmunoblastic T cell lymphoma. In some embodiments, the T cell lymphoma is extranodal NK/T cell lymphoma, nasal type. In some embodiments, the T cell lymphoma is enteropathy-associated intestinal T cell lymphoma. In some embodiments, the T cell lymphoma is anaplastic large cell lymphoma.

In some embodiments, there is provided a method of treating T cell lymphoma in an individual (such as a human) comprising administering to the individual a) an effective amount of a composition comprising nanoparticles comprising sirolimus or a derivative thereof and an albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 14 mg/m² romidepsin. In some embodiments, the method further comprises administering to the individual at least one therapeutic agent used in a standard combination therapy with romidepsin. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the romidepsin is administered intravenously. In some embodiments, the T cell lymphoma is recurrent T cell lymphoma. In some embodiments, the T cell lymphoma is refractory to one or more drugs used in a standard therapy for T cell lymphoma, such as, but not limited to, interferon, zidovudine, cyclophosphamide, doxorubicin, vincristine, prednisone, cisplatin, etoposide, ifosfamide, carboplatin, dexamethasone, methotrexate, brentuximab vedotin, pralatrexate, bortezomib, belinostat, alemtuzumab, denileukin diftitox, and romidepsin. In some embodiments, the T cell lymphoma is selected from the group consisting of cutaneous T cell lymphoma (such as mycosis fungoides and Sezary syndrome), angioimmunoblastic T cell lymphoma, extranodal NK/T cell lymphoma, nasal type, enteropathy-associated intestinal T cell lymphoma (EATL), and anaplastic large cell lymphoma (ALCL). In some embodiments, the T cell lymphoma is cutaneous T cell lymphoma. In some embodiments, the T cell lymphoma is angioimmunoblastic T cell lymphoma. In some embodiments, the T cell lymphoma is extranodal NK/T cell lymphoma, nasal type. In some embodiments, the T cell lymphoma is enteropathy-associated intestinal T cell lymphoma. In some embodiments, the T cell lymphoma is anaplastic large cell lymphoma.

In some embodiments, according to any of the methods of treating T cell lymphoma in an individual described herein, the individual is a human who exhibits one or more symptoms associated with T cell lymphoma. In some embodiments, the individual is at an early stage of T cell lymphoma. In some embodiments, the individual is at an advanced stage of T cell lymphoma. In some of embodiments, the individual is genetically or otherwise predisposed (e.g., having a risk factor) to developing T cell lymphoma. Individuals at risk for T cell lymphoma include, e.g., those having relatives who have experienced T cell lymphoma, and those whose risk is determined by analysis of genetic or biochemical markers. In some embodiments, the individual may be a human who has a gene, genetic mutation, or polymorphism associated with T cell lymphoma (e.g., NPM1, ALK, t(2;5)) or has one or more extra copies of a gene associated with T cell lymphoma. In some embodiments, the individual has chromosomal translocation t(2;5) (such as t(2;5)(p23;q35)). In some embodiments, the cancer cells express an NPM1-ALK fusion protein. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Chronic Myeloid Leukemia

In some embodiments, there is provided a method of treating chronic myeloid leukemia in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. Chronic myeloid leukemia includes, but is not limited to, chronic phase CML, accelerated phase CML, and blast crisis CML. In some embodiments, the chronic myeloid leukemia is chronic phase CML. In some embodiments, the chronic myeloid leukemia is accelerated phase CML. In some embodiments, the chronic myeloid leukemia is blast crisis CML. In some embodiments, the chronic myeloid leukemia is relapsed or refractory to standard therapy.

In some embodiments, there is provided a method of treating chronic myeloid leukemia in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the chronic myeloid leukemia is recurrent chronic myeloid leukemia. In some embodiments, the chronic myeloid leukemia is refractory to one or more drugs used in a standard therapy for chronic myeloid leukemia, such as, but not limited to, cytarabine, hydroxyurea, interferon alfa-2b, imatinib, dasatinib, and nilotinib. In some embodiments, the chronic myeloid leukemia is selected from the group consisting of chronic phase CML, accelerated phase CML, and blast crisis CML. In some embodiments, the chronic myeloid leukemia is chronic phase CML. In some embodiments, the chronic myeloid leukemia is accelerated phase CML. In some embodiments, the chronic myeloid leukemia is blast crisis CML.

In some embodiments, there is provided a method of treating chronic myeloid leukemia in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of nilotinib. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of nilotinib. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of nilotinib. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of nilotinib. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of nilotinib. In some embodiments, the method further comprises administering to the individual at least one therapeutic agent used in a standard combination therapy with nilotinib. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the chronic myeloid leukemia is recurrent chronic myeloid leukemia. In some embodiments, the chronic myeloid leukemia is refractory to one or more drugs used in a standard therapy for chronic myeloid leukemia, such as, but not limited to, cytarabine, hydroxyurea, interferon alfa-2b, imatinib, dasatinib, and nilotinib. In some embodiments, the chronic myeloid leukemia is selected from the group consisting of chronic phase CML, accelerated phase CML, and blast crisis CML. In some embodiments, the chronic myeloid leukemia is chronic phase CML. In some embodiments, the chronic myeloid leukemia is accelerated phase CML. In some embodiments, the chronic myeloid leukemia is blast crisis CML.

In some embodiments, there is provided a method of treating chronic myeloid leukemia in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the chronic myeloid leukemia is recurrent chronic myeloid leukemia. In some embodiments, the chronic myeloid leukemia is refractory to one or more drugs used in a standard therapy for chronic myeloid leukemia, such as, but not limited to, cytarabine, hydroxyurea, interferon alfa-2b, imatinib, dasatinib, and nilotinib. In some embodiments, the chronic myeloid leukemia is selected from the group consisting of chronic phase CML, accelerated phase CML, and blast crisis CML. In some embodiments, the chronic myeloid leukemia is chronic phase CML. In some embodiments, the chronic myeloid leukemia is accelerated phase CML. In some embodiments, the chronic myeloid leukemia is blast crisis CML.

In some embodiments, there is provided a method of treating chronic myeloid leukemia in an individual (such as a human) comprising administering to the individual a) an effective amount of a composition comprising nanoparticles comprising sirolimus or a derivative thereof and an albumin; and b) an effective amount of nilotinib. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of nilotinib. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of nilotinib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of nilotinib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of nilotinib. In some embodiments, the method further comprises administering to the individual at least one therapeutic agent used in a standard combination therapy with nilotinib. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the chronic myeloid leukemia is recurrent chronic myeloid leukemia. In some embodiments, the chronic myeloid leukemia is refractory to one or more drugs used in a standard therapy for chronic myeloid leukemia, such as, but not limited to, cytarabine, hydroxyurea, interferon alfa-2b, imatinib, dasatinib, and nilotinib. In some embodiments, the chronic myeloid leukemia is selected from the group consisting of chronic phase CML, accelerated phase CML, and blast crisis CML. In some embodiments, the chronic myeloid leukemia is chronic phase CML. In some embodiments, the chronic myeloid leukemia is accelerated phase CML. In some embodiments, the chronic myeloid leukemia is blast crisis CML.

In some embodiments, there is provided a method of treating chronic myeloid leukemia in an individual (such as a human) comprising administering to the individual a) an effective amount of a composition comprising nanoparticles comprising sirolimus or a derivative thereof and an albumin, wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 200 to about 400 mg bi-daily (including for example about any of 200, 220, 240, 260, 280, 300, 320, 340, 360, 380, or 400 mg bi-daily, including any range between these values) nilotinib. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 200 to about 400 mg bi-daily (including for example about any of 200, 220, 240, 260, 280, 300, 320, 340, 360, 380, or 400 mg bi-daily, including any range between these values) nilotinib. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 200 to about 400 mg bi-daily (including for example about any of 200, 220, 240, 260, 280, 300, 320, 340, 360, 380, or 400 mg bi-daily, including any range between these values) nilotinib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 200 to about 400 mg bi-daily (including for example about any of 200, 220, 240, 260, 280, 300, 320, 340, 360, 380, or 400 mg bi-daily, including any range between these values) nilotinib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 200 to about 400 mg bi-daily (including for example about any of 200, 220, 240, 260, 280, 300, 320, 340, 360, 380, or 400 mg bi-daily, including any range between these values) nilotinib. In some embodiments, the method further comprises administering to the individual at least one therapeutic agent used in a standard combination therapy with nilotinib. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the nilotinib is administered orally. In some embodiments, the chronic myeloid leukemia is recurrent chronic myeloid leukemia. In some embodiments, the chronic myeloid leukemia is refractory to one or more drugs used in a standard therapy for chronic myeloid leukemia, such as, but not limited to, cytarabine, hydroxyurea, interferon alfa-2b, imatinib, dasatinib, and nilotinib. In some embodiments, the chronic myeloid leukemia is selected from the group consisting of chronic phase CML, accelerated phase CML, and blast crisis CML. In some embodiments, the chronic myeloid leukemia is chronic phase CML. In some embodiments, the chronic myeloid leukemia is accelerated phase CML. In some embodiments, the chronic myeloid leukemia is blast crisis CML.

In some embodiments, there is provided a method of treating chronic myeloid leukemia in an individual (such as a human) comprising administering to the individual a) an effective amount of a composition comprising nanoparticles comprising sirolimus or a derivative thereof and an albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily nilotinib. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily nilotinib. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily nilotinib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily nilotinib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily nilotinib. In some embodiments, the method further comprises administering to the individual at least one therapeutic agent used in a standard combination therapy with nilotinib. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the nilotinib is administered orally. In some embodiments, the chronic myeloid leukemia is recurrent chronic myeloid leukemia. In some embodiments, the chronic myeloid leukemia is refractory to one or more drugs used in a standard therapy for chronic myeloid leukemia, such as, but not limited to, cytarabine, hydroxyurea, interferon alfa-2b, imatinib, dasatinib, and nilotinib. In some embodiments, the chronic myeloid leukemia is selected from the group consisting of chronic phase CML, accelerated phase CML, and blast crisis CML. In some embodiments, the chronic myeloid leukemia is chronic phase CML. In some embodiments, the chronic myeloid leukemia is accelerated phase CML. In some embodiments, the chronic myeloid leukemia is blast crisis CML.

In some embodiments, according to any of the methods of treating chronic myeloid leukemia in an individual described herein, the individual is a human who exhibits one or more symptoms associated with chronic myeloid leukemia. In some embodiments, the individual is at an early stage of chronic myeloid leukemia. In some embodiments, the individual is at an advanced stage of chronic myeloid leukemia. In some of embodiments, the individual is genetically or otherwise predisposed (e.g., having a risk factor) to developing chronic myeloid leukemia. Individuals at risk for chronic myeloid leukemia include, e.g., those having relatives who have experienced chronic myeloid leukemia, and those whose risk is determined by analysis of genetic or biochemical markers. In some embodiments, the individual may be a human who has a gene, genetic mutation, or polymorphism associated with chronic myeloid leukemia (e.g., ABL1, BCR, JAK2, TEL, t(9;12)(p24;p13), t(9;22)(q34;q11)) or has one or more extra copies of a gene associated with chronic myeloid leukemia. In some embodiments, the individual has the chromosomal translocation t(9;12)(p24;p13). In some embodiments, the individual has the chromosomal translocation t(9;22)(q34;q11). In some embodiments, the cancer cells express a BCR-ABL1 fusion protein. In some embodiments, the cancer cells express a TEL-JAK2 fusion protein. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Acute Myeloid Leukemia

In some embodiments, there is provided a method of treating acute myeloid leukemia in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. Acute myeloid leukemia includes, but is not limited to, undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In some embodiments, the acute myeloid leukemia is undifferentiated AML (M0). In some embodiments, the acute myeloid leukemia is myeloblastic leukemia (M1). In some embodiments, the acute myeloid leukemia is myeloblastic leukemia (M2). In some embodiments, the acute myeloid leukemia is promyelocytic leukemia (M3 or M3 variant [M3V]). In some embodiments, the acute myeloid leukemia is myelomonocytic leukemia (M4 or M4 variant with eosinophilia [M4E]). In some embodiments, the acute myeloid leukemia is monocytic leukemia (M5). In some embodiments, the acute myeloid leukemia is erythroleukemia (M6). In some embodiments, the acute myeloid leukemia is megakaryoblastic leukemia (M7). In some embodiments, the acute myeloid leukemia is relapsed or refractory to standard therapy.

In some embodiments, there is provided a method of treating acute myeloid leukemia in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the acute myeloid leukemia is recurrent acute myeloid leukemia. In some embodiments, the acute myeloid leukemia is refractory to one or more drugs used in a standard therapy for acute myeloid leukemia, such as, but not limited to, fludarabine, decitabine, cytarabine, busulfan, azacitidine, idarubicin, and daunorubicin. In some embodiments, the acute myeloid leukemia is selected from the group consisting of undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In some embodiments, the acute myeloid leukemia is undifferentiated AML (M0). In some embodiments, the acute myeloid leukemia is myeloblastic leukemia (M1). In some embodiments, the acute myeloid leukemia is myeloblastic leukemia (M2). In some embodiments, the acute myeloid leukemia is promyelocytic leukemia (M3 or M3 variant [M3V]). In some embodiments, the acute myeloid leukemia is myelomonocytic leukemia (M4 or M4 variant with eosinophilia [M4E]). In some embodiments, the acute myeloid leukemia is monocytic leukemia (M5). In some embodiments, the acute myeloid leukemia is erythroleukemia (M6). In some embodiments, the acute myeloid leukemia is megakaryoblastic leukemia (M7).

In some embodiments, there is provided a method of treating acute myeloid leukemia in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of sorafenib. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of sorafenib. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of sorafenib. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of sorafenib. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of sorafenib. In some embodiments, the method further comprises administering to the individual at least one therapeutic agent used in a standard combination therapy with sorafenib. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the acute myeloid leukemia is recurrent acute myeloid leukemia. In some embodiments, the acute myeloid leukemia is refractory to one or more drugs used in a standard therapy for acute myeloid leukemia, such as, but not limited to, fludarabine, decitabine, cytarabine, busulfan, azacitidine, idarubicin, and daunorubicin. In some embodiments, the acute myeloid leukemia is selected from the group consisting of undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In some embodiments, the acute myeloid leukemia is undifferentiated AML (M0). In some embodiments, the acute myeloid leukemia is myeloblastic leukemia (M1). In some embodiments, the acute myeloid leukemia is myeloblastic leukemia (M2). In some embodiments, the acute myeloid leukemia is promyelocytic leukemia (M3 or M3 variant [M3V]). In some embodiments, the acute myeloid leukemia is myelomonocytic leukemia (M4 or M4 variant with eosinophilia [M4E]). In some embodiments, the acute myeloid leukemia is monocytic leukemia (M5). In some embodiments, the acute myeloid leukemia is erythroleukemia (M6). In some embodiments, the acute myeloid leukemia is megakaryoblastic leukemia (M7).

In some embodiments, there is provided a method of treating acute myeloid leukemia in an individual (such as a human) comprising administering to the individual a) an effective amount of a composition comprising nanoparticles comprising sirolimus or a derivative thereof and an albumin; and b) an effective amount of a kinase inhibitor (such as sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the acute myeloid leukemia is recurrent acute myeloid leukemia. In some embodiments, the acute myeloid leukemia is refractory to one or more drugs used in a standard therapy for acute myeloid leukemia, such as, but not limited to, fludarabine, decitabine, cytarabine, busulfan, azacitidine, idarubicin, and daunorubicin. In some embodiments, the acute myeloid leukemia is selected from the group consisting of undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In some embodiments, the acute myeloid leukemia is undifferentiated AML (M0). In some embodiments, the acute myeloid leukemia is myeloblastic leukemia (M1). In some embodiments, the acute myeloid leukemia is myeloblastic leukemia (M2). In some embodiments, the acute myeloid leukemia is promyelocytic leukemia (M3 or M3 variant [M3V]). In some embodiments, the acute myeloid leukemia is myelomonocytic leukemia (M4 or M4 variant with eosinophilia [M4E]). In some embodiments, the acute myeloid leukemia is monocytic leukemia (M5). In some embodiments, the acute myeloid leukemia is erythroleukemia (M6). In some embodiments, the acute myeloid leukemia is megakaryoblastic leukemia (M7).

In some embodiments, there is provided a method of treating acute myeloid leukemia in an individual (such as a human) comprising administering to the individual a) an effective amount of a composition comprising nanoparticles comprising sirolimus or a derivative thereof and an albumin; and b) an effective amount of sorafenib. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of sorafenib. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of sorafenib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of sorafenib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of sorafenib. In some embodiments, the method further comprises administering to the individual at least one therapeutic agent used in a standard combination therapy with sorafenib. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the acute myeloid leukemia is recurrent acute myeloid leukemia. In some embodiments, the acute myeloid leukemia is refractory to one or more drugs used in a standard therapy for acute myeloid leukemia, such as, but not limited to, fludarabine, decitabine, cytarabine, busulfan, azacitidine, idarubicin, and daunorubicin. In some embodiments, the acute myeloid leukemia is selected from the group consisting of undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In some embodiments, the acute myeloid leukemia is undifferentiated AML (M0). In some embodiments, the acute myeloid leukemia is myeloblastic leukemia (M1). In some embodiments, the acute myeloid leukemia is myeloblastic leukemia (M2). In some embodiments, the acute myeloid leukemia is promyelocytic leukemia (M3 or M3 variant [M3V]). In some embodiments, the acute myeloid leukemia is myelomonocytic leukemia (M4 or M4 variant with eosinophilia [M4E]). In some embodiments, the acute myeloid leukemia is monocytic leukemia (M5). In some embodiments, the acute myeloid leukemia is erythroleukemia (M6). In some embodiments, the acute myeloid leukemia is megakaryoblastic leukemia (M7).

In some embodiments, there is provided a method of treating acute myeloid leukemia in an individual (such as a human) comprising administering to the individual a) an effective amount of a composition comprising nanoparticles comprising sirolimus or a derivative thereof and an albumin, wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 250 to about 400 mg bi-daily (including for example about any of 250, 275, 300, 325, 350, 375, or 400 mg bi-daily, including any range between these values) sorafenib. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 250 to about 400 mg bi-daily (including for example about any of 250, 275, 300, 325, 350, 375, or 400 mg bi-daily, including any range between these values) sorafenib. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 250 to about 400 mg bi-daily (including for example about any of 250, 275, 300, 325, 350, 375, or 400 mg bi-daily, including any range between these values) sorafenib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 250 to about 400 mg bi-daily (including for example about any of 250, 275, 300, 325, 350, 375, or 400 mg bi-daily, including any range between these values) sorafenib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1), and wherein the sirolimus or derivative thereof is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², and any ranges between these values); and b) about 250 to about 400 mg bi-daily (including for example about any of 250, 275, 300, 325, 350, 375, or 400 mg bi-daily, including any range between these values) sorafenib. In some embodiments, the method further comprises administering to the individual at least one therapeutic agent used in a standard combination therapy with sorafenib. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the sorafenib is administered orally. In some embodiments, the acute myeloid leukemia is recurrent acute myeloid leukemia. In some embodiments, the acute myeloid leukemia is refractory to one or more drugs used in a standard therapy for acute myeloid leukemia, such as, but not limited to, fludarabine, decitabine, cytarabine, busulfan, azacitidine, idarubicin, and daunorubicin. In some embodiments, the acute myeloid leukemia is selected from the group consisting of undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In some embodiments, the acute myeloid leukemia is undifferentiated AML (M0). In some embodiments, the acute myeloid leukemia is myeloblastic leukemia (M1). In some embodiments, the acute myeloid leukemia is myeloblastic leukemia (M2). In some embodiments, the acute myeloid leukemia is promyelocytic leukemia (M3 or M3 variant [M3V]). In some embodiments, the acute myeloid leukemia is myelomonocytic leukemia (M4 or M4 variant with eosinophilia [M4E]). In some embodiments, the acute myeloid leukemia is monocytic leukemia (M5). In some embodiments, the acute myeloid leukemia is erythroleukemia (M6). In some embodiments, the acute myeloid leukemia is megakaryoblastic leukemia (M7).

In some embodiments, there is provided a method of treating acute myeloid leukemia in an individual (such as a human) comprising administering to the individual a) an effective amount of a composition comprising nanoparticles comprising sirolimus or a derivative thereof and an albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily sorafenib. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin, wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily sorafenib. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily sorafenib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily sorafenib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1), wherein the amount of the sirolimus or derivative thereof in the composition is about 45 mg/m² to about 100 mg/m² (including for example about any of 45 mg/m², about 75 mg/m², and about 100 mg/m²), and wherein the composition is administered on days 1, 8, and 15 of a 28-day cycle for at least one (such as at least about any of 2, 3, 4, 5, 6, 7, 8, 9, 10, or more) cycle; and b) about 400 mg bi-daily sorafenib. In some embodiments, the method further comprises administering to the individual at least one therapeutic agent used in a standard combination therapy with sorafenib. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the sirolimus nanoparticle composition is administered intravenously. In some embodiments, the sirolimus nanoparticle composition is administered subcutaneously. In some embodiments, the sorafenib is administered orally. In some embodiments, the acute myeloid leukemia is recurrent acute myeloid leukemia. In some embodiments, the acute myeloid leukemia is refractory to one or more drugs used in a standard therapy for acute myeloid leukemia, such as, but not limited to, fludarabine, decitabine, cytarabine, busulfan, azacitidine, idarubicin, and daunorubicin. In some embodiments, the acute myeloid leukemia is selected from the group consisting of undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In some embodiments, the acute myeloid leukemia is undifferentiated AML (M0). In some embodiments, the acute myeloid leukemia is myeloblastic leukemia (M1). In some embodiments, the acute myeloid leukemia is myeloblastic leukemia (M2). In some embodiments, the acute myeloid leukemia is promyelocytic leukemia (M3 or M3 variant [M3V]). In some embodiments, the acute myeloid leukemia is myelomonocytic leukemia (M4 or M4 variant with eosinophilia [M4E]). In some embodiments, the acute myeloid leukemia is monocytic leukemia (M5). In some embodiments, the acute myeloid leukemia is erythroleukemia (M6). In some embodiments, the acute myeloid leukemia is megakaryoblastic leukemia (M7).

In some embodiments, according to any of the methods of treating acute myeloid leukemia in an individual described herein, the individual is a human who exhibits one or more symptoms associated with acute myeloid leukemia. In some embodiments, the individual is at an early stage of acute myeloid leukemia. In some embodiments, the individual is at an advanced stage of acute myeloid leukemia. In some of embodiments, the individual is genetically or otherwise predisposed (e.g., having a risk factor) to developing acute myeloid leukemia. Individuals at risk for acute myeloid leukemia include, e.g., those having relatives who have experienced acute myeloid leukemia, and those whose risk is determined by analysis of genetic or biochemical markers. In some embodiments, the individual may be a human who has a gene, genetic mutation, or polymorphism associated with acute myeloid leukemia (e.g., ETO, AML1, TEL, TrkC, t(8;21)(q22;q22), t(12;15)(p13;q25), or t(1;12)(q21;p13)) or has one or more extra copies of a gene associated with acute myeloid leukemia. In some embodiments, the individual has the chromosomal translocation t(8;21)(q22;q22). In some embodiments, the individual has the chromosomal translocation t(12;15)(p13;q25). In some embodiments, the individual has the chromosomal translocation t(1;12)(q21;p13). In some embodiments, the cancer cells express an ETO-AML1 fusion protein. In some embodiments, the cancer cells express a TEL-TrkC fusion protein. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

Also provided are pharmaceutical compositions comprising nanoparticles comprising an mTOR inhibitor (such as a limus drug, e.g., sirolimus or a derivative thereof) and/or a second therapeutic agent for use in any of the methods of treating a hematological malignancy described herein. In some embodiments, the pharmaceutical composition comprises nanoparticles comprising an mTOR inhibitor (such as a limus drug, e.g., sirolimus or a derivative thereof) and albumin (such as human albumin). In some embodiments, the pharmaceutical composition comprises a second therapeutic agent. In some embodiments, the pharmaceutical composition comprises a) nanoparticles comprising an mTOR inhibitor (such as a limus drug, e.g., sirolimus or a derivative thereof) and albumin (such as human albumin); and b) a second therapeutic agent. In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen.

### Pharmaceutical Compositions

The nanoparticle compositions (such as mTOR inhibitor nanoparticle compositions) and/or second therapeutic agents described herein can be used in the preparation of a formulation, such as a pharmaceutical composition, by combining the nanoparticle composition(s) or second therapeutic agent(s) described above with a pharmaceutically acceptable carrier, an excipient, a stabilizing agent, and/or another agent known in the art for use in the methods of treatment, methods of administration, and dosage regimes described herein.

To increase stability by increasing the negative zeta potential of nanoparticles in a pharmaceutical composition, certain negatively charged components can be added. Such negatively charged components include, but are not limited to, bile salts, bile acids, glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid, and others; and phospholipids including lecithin (egg yolk) based phospholipids, which includes the following phosphatidylcholines: palmitoyloleoylphosphatidylcholine, palmitoyllinoleoylphosphatidylcholine, stearoyllinoleoylphosphatidylcholine, stearoyloleoylphosphatidylcholine, stearoylarachidoylphosphatidylcholine, and dipalmitoylphosphatidylcholine. Other phospholipids include L-α-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the pharmaceutical composition is suitable for administration to a human. In some embodiments, the pharmaceutical composition is suitable for administration to a mammal, such as, in the veterinary context, domestic pets and agricultural animals. There are a wide variety of suitable formulations of the inventive composition (see, e.g., U.S. Pat. Nos. 5,916,596 and 6,096,331, which are hereby incorporated by reference in their entireties). The following formulations and methods are merely exemplary and are in no way limiting. Formulations suitable for oral administration can comprise (a) liquid solutions, such as an effective amount of the active ingredient (e.g., nanoparticle composition or second therapeutic agent) dissolved in diluents, such as water, saline, or orange juice, (b) capsules, sachets or tablets, each containing a predetermined amount of the active ingredient, as solids or granules, (c) suspensions in an appropriate liquid, (d) suitable emulsions, and (e) powders. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a freeze-dried (lyophilized) condition requiring only the addition of a sterile liquid excipient (e.g., water) for injection, immediately prior to use. Extemporaneous injection solutions and suspensions can be prepared from sterile powders, granules, and tablets of the kind previously described.

Formulations suitable for aerosol administration are provided that comprise the inventive compositions described above. In some embodiments, the formulation suitable for aerosol administration is an aqueous or non-aqueous isotonic sterile solutions, and can contain anti-oxidants, buffers, bacteriostats, and/or solutes. In some embodiments, the formulation suitable for aerosol administration is an aqueous or non-aqueous sterile suspensions that can include suspending agents, solubilizers, thickening agents, stabilizing agents, and/or preservatives, alone or in combination with other suitable components. These aerosol formulations can be placed into pressurized acceptable propellants, such as dichlorodifluoromethane, propane, nitrogen, and the like. They can also be formulated as pharmaceuticals for non-pressured preparations, such as for use in a nebulizer or an atomizer.

In some embodiments, the pharmaceutical composition is formulated to have a pH in the range of about 4.5 to about 9.0, including for example pH ranges of any of about 5.0 to about 8.0, about 6.5 to about 7.5, and about 6.5 to about 7.0. In some embodiments, the pH of the pharmaceutical composition is formulated to no less than about 6, including for example no less than about any of 6.5, 7, or 8 (e.g., about 8). The pharmaceutical composition can also be made to be isotonic with blood by the addition of a suitable tonicity modifier, such as glycerol.

The nanoparticles of this invention can be enclosed in a hard or soft capsule, can be compressed into tablets, or can be incorporated with beverages or food or otherwise incorporated into the diet. Capsules can be formulated by mixing the nanoparticles with an inert pharmaceutical diluent and inserting the mixture into a hard gelatin capsule of the appropriate size. If soft capsules are desired, a slurry of the nanoparticles with an acceptable vegetable oil, light petroleum or other inert oil can be encapsulated by machine into a gelatin capsule.

Also provided are unit dosage forms comprising the compositions and formulations described herein. These unit dosage forms can be stored in a suitable packaging in single or multiple unit dosages and may also be further sterilized and sealed. For example, the pharmaceutical composition (e.g., a dosage or unit dosage form of a pharmaceutical composition) may include (i) nanoparticles that comprise sirolimus or a derivative thereof and an albumin and (ii) a pharmaceutically acceptable carrier. In other examples, the pharmaceutical composition (e.g., a dosage or unit dosage form of a pharmaceutical composition includes a) nanoparticles comprising sirolimus or a derivative thereof and an albumin and b) at least one other therapeutic agent. In some embodiments, the other therapeutic agent comprises any of the second therapeutic agents described herein). In some embodiments, the pharmaceutical composition also includes one or more other compounds (or pharmaceutically acceptable salts thereof) that are useful for treating cancer. In some embodiments, the amount of mTOR inhibitor (such as a limus drug, e.g., sirolimus or a derivative thereof) in the composition is included in any of the following ranges: about 20 to about 50 mg, about 50 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, or about 300 to about 350 mg. In some embodiments, the amount of mTOR inhibitor (such as a limus drug, e.g., sirolimus or a derivative thereof) in the composition (e.g., a dosage or unit dosage form) is in the range of about 54 mg to about 540 mg, such as about 180 mg to about 270 mg or about 216 mg, of the mTOR inhibitor. In some embodiments, the carrier is suitable for parental administration (e.g., intravenous administration). In some embodiments, a taxane is not contained in the composition. In some embodiments, the mTOR inhibitor (such as a limus drug, e.g., sirolimus or a derivative thereof) is the only pharmaceutically active agent for the treatment of solid tumors that is contained in the composition.

Thus, in some embodiments, there is provided a pharmaceutical composition according to any of the pharmaceutical compositions described above comprising nanoparticles comprising an mTOR inhibitor (such as a limus drug, e.g., sirolimus or a derivative thereof) and/or a second therapeutic agent for use in any of the methods of treating a solid tumor described herein. In some embodiments, the pharmaceutical composition comprises nanoparticles comprising an mTOR inhibitor (such as a limus drug, e.g., sirolimus or a derivative thereof) and albumin (such as human albumin) In some embodiments, the pharmaceutical composition comprises a second therapeutic agent. In some embodiments, the pharmaceutical composition comprises a) nanoparticles comprising an mTOR inhibitor (such as a limus drug, e.g., sirolimus or a derivative thereof) and albumin (such as human albumin); and b) a second therapeutic agent. In some embodiments, the second therapeutic agent is an immunomodulator. In some embodiments, the second therapeutic agent is an immunostimulator. In some embodiments, the second therapeutic agent is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the second therapeutic agent is an immunomodulator selected from the group consisting of pomalidomide and lenalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen (TAA). In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

### Diseases to be Treated

In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent.

Hematologic malignancies are cancers of the blood or bone marrow. Examples of hematological (or hematogenous) malignancies include leukemias, including acute leukemias (such as acute lymphocytic leukemia, acute myelocytic leukemia, acute myeloid leukemia and myeloblastic, promyelocytic, myelomonocytic, monocytic and erythroleukemia), chronic leukemias (such as chronic myelocytic (granulocytic) leukemia, chronic myeloid leukemia, and chronic lymphocytic leukemia), polycythemia vera, B cell lymphoma (such as splenic marginal zone lymphoma, extranodal marginal zone B cell lymphoma, nodal marginal zone B cell lymphoma, follicular lymphoma, primary cutaneous follicle center lymphoma, mantle cell lymphoma, diffuse large B cell lymphoma, lymphomatoid granulomatosis, primary mediastinal large B cell lymphoma, intravascular large B cell lymphoma, ALK+ large B cell lymphoma, plasmablastic lymphoma, primary effusion lymphoma, and Burkitt lymphoma), T cell and/or NK cell lymphoma (such as adult T cell lymphoma, extranodal NK/T cell lymphoma, enteropathy-associated T cell lymphoma, hepatosplenic T cell lymphoma, blastic NK cell lymphoma, primary cutaneous anaplastic large cell lymphoma, lymphomatoid papulosis, peripheral T cell lymphoma, angioimmunoblastic T cell lymphoma, and anaplastic large cell lymphoma), Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

### Multiple Myeloma

Multiple myeloma (MM), a B cell malignancy characterized by the accumulation of plasma cells in the bone marrow and the secretion of large amounts of monoclonal antibodies that ultimately causes bone lesions, hypercalcaemia, renal disease, anemia, and immunodeficiency (Raab M S, Podar K, Breitkreutz I, Richardson P G, Anderson K C., Lancet 374:324-39, 2009), is the second most frequent blood disease in the United States affecting 7.1 per 100,000 men and 4.6 per 100,000 women.

MM is characterized by monoclonal proliferation of malignant plasma cells (PCs) in the bone marrow, the presence of high levels of monoclonal serum antibody, the development of osteolytic bone lesions, and the induction of angiogenesis, neutropenia, amyloidosis, and hypercalcemia (Vanderkerken K, Asosingh K, Croucher P, Van Camp B., Immunol Rev 194:196-206, 2003; Raab M S, Podar K, Breitkreutz I, Richardson P G, Anderson K C., Lancet 374:324-39, 2009). MM is seen as a multistep transformation process. (G. Pratt., J. Clin. Pathol: Molec. Pathol. 55: 273-83, 2002). Although little is known about the immortalizing and initial transforming events, the initial event is thought to be the immortalization of a plasma cell to form a clone, which may be quiescent, non-accumulating and not cause end organ damage due to accumulation of plasma cells within the bone marrow (MGUS). Smoldering MM (SMM) also has no detectable end-organ damage, but differs from MGUS by having a serum mlg level higher than 3 g/dl or a BM P C content of more than 10% and an average rate of progression to symptomatic MM of 10% per year. Currently there are no tests that measure phenotypic or genotypic markers on tumor cells that predict progression. (W. Michael Kuehl and P. Leif Bergsagel, J. Clin. Invest. 122 (10): 3456-63, 2012). An abnormal immunophenotype distinguishes healthy plasma cells (PCs) from tumor cells. Healthy BM PCs are CD38+CD138+CD19+CD45+CD56−. Id. Although MM tumor cells also are CD38+CD138+, 90% are CD19−, 99% are CD45− or CD45 lo, and 70% are CD56+. Id.

The prognosis and treatment of this disease has greatly evolved over the past decade due to the incorporation of new agents that act as immunomodulators and proteasome inhibitors. Despite recent progress with a number of novel treatments (Raab M S, Podar K, Breitkreutz I, Richardson P G, Anderson K C., Lancet 374:324-39, 2009; Schwartz R N, Vozniak M., J. Manag. Care Pharm. 14:12-19, 2008), patients only experience somewhat longer periods of remission. Because of the development of drug resistance or relapse, MM is an incurable disease (Schwartz R N, Vozniak M., J. Manag. Care Pharm. 14:12-9, 2008; Kyle R A., Blood 111:4417-8, 2008), with a median survival time of 3-4 years.

Disease management is currently tailored based on the patient's co-morbidity factors and stage of disease (for a complete list of treatments and their implementation, see Raab M S, Podar K, Breitkreutz I, Richardson P G, Anderson K C., Lancet 374:324-39, 2009, and Schwartz R N, Vozniak M., J. Manag. Care Pharm. 14:12-9, 2008).

### Chronic Myeloid Leukemia

Chronic myeloid (or myelogenous or myelocytic) leukemia (CML), also known as chronic granulocytic leukemia (CGL), is a cancer of the white blood cells. It is a hematological stem cell disorder caused by increased and unregulated growth of myeloid cells in the bone marrow, and the accumulation of excessive white blood cells. CML is associated with a characteristic chromosomal translocation called the Philadelphia chromosome, and was the first cancer to be linked to a clear genetic abnormality (Nowell P C, J. Clin. Investigation 117(8):2033-2035, 2007). 95% of CML patients have the ABL gene from chromosome 9 fused with the breakpoint cluster (BCR) gene from chromosome 22, resulting in the Philadelphia chromosome. This Philadelphia chromosome is responsible for the production of the BCR-ABL fusion protein, a constitutively active tyrosine kinase that causes uncontrolled cellular proliferation. An ABL inhibitor, imatinib, was approved by the FDA for the treatment of CML, and is currently used as first-line therapy. It has been reported that 80% of CML patients respond to imatinib with under 3% progressing to advanced disease within 5 years. The durability of clinical response, however, is adversely affected by the development of resistance to drug therapy. During the last decade, major progress has been made in the treatment of CML, by the clinical use of tyrosine kinase inhibitors (TKI) which have transformed the prognosis of the disease and prolonged survival. In Western countries it accounts for 15-20% of all adult leukemias and 14% of leukemias overall (including the pediatric population).

CML is often divided into three phases based on clinical characteristics and laboratory findings. In the absence of intervention, CML typically begins in the chronic phase, and over the course of several years progresses to an accelerated phase and ultimately to a blast crisis. Blast crisis is the terminal phase of CML and clinically behaves like an acute leukemia. Drug treatment will usually stop this progression if started early. One of the drivers of the progression from chronic phase through acceleration and blast crisis is the acquisition of new chromosomal abnormalities (in addition to the Philadelphia chromosome). (Faderl et al., Annals of Internal Medicine 131(3):207-219, 1999). Some patients may already be in the accelerated phase or blast crisis by the time they are diagnosed (Tefferi A, Hematology Am. Soc. Hematol. Educ. Program. 2006(1):240-245, 2006).

### Acute Myeloid Leukemia

Acute leukemias are divided into lymphoblastic (ALL) and nonlymphoblastic (ANLL) types. The Merck Manual, 946-949 (17^{th} ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease which is established by laboratory findings and bone marrow examination. ANLL, also known as acute myeloid (or myelogenous or myeloblastic) leukemia (AML), occurs at all ages and is the more common acute leukemia among adults; it is the form usually associated with irradiation as a causative agent.

### Mantle Cell Lymphoma

Mantle cell lymphoma (MCL) is a type of non-Hodgkin's lymphoma (NHL), comprising about 6% of NHL cases (Skarbnik A P & Goy A H, Clin Adv Hematol Oncol 13(1):44-55, 2015). MCL is a subtype of B-cell lymphoma, resulting from CD5-positive antigen-naive pregerminal center B-cells within the mantle zone that surrounds normal germinal center follicles. MCL cells generally over-express cyclin D1 due to a t(11:14) chromosomal translocation (Li J Y et al., Am. J. Pathol. 154(5):1449-52, 1999; Barouk-Simonet E. et al., Ann. Genet. 45(3):165-8, 2002).

MCL, like most malignancies, results from the acquisition of a combination of genetic mutations in somatic cells. This leads to a clonal expansion of malignant B lymphocytes. The factors that initiate the genetic alterations are typically not identifiable, and usually occur in people with no particular risk factors for lymphoma development. Because it is an acquired genetic disorder, MCL is neither communicable nor inheritable. A defining characteristic of MCL is mutation and overexpression of cyclin D1, a cell cycle gene, that contributes to the abnormal proliferation of the malignant cells. MCL cells may also be resistant to drug induced apoptosis, making them harder to cure with chemotherapy or radiation. Cells affected by MCL proliferate in a nodular or diffuse pattern with two main cytologic variants: typical or blastic. Typical cases are small to intermediate sized cells with irregular nuclei. Blastic (aka blastoid) variants have intermediate to large sized cells with finely dispersed chromatin and are more aggressive in nature. The tumor cells accumulate in the lymphoid system, including lymph nodes and the spleen, with non-useful cells eventually rendering the system dysfunctional. MCL may also replace normal cells in the bone marrow, which impairs normal blood cell production.

### T-Cell Lymphoma

The T-cell lymphomas include four types of lymphomas that affect T cells. These account for about one in ten cases of non-Hodgkin lymphoma. The four classes of T-cell lymphomas are extranodal NK/T-cell lymphoma, nasal type (angiocentric T-cell lymphoma), cutaneous T-cell lymphoma, anaplastic large cell lymphoma, and angioimmunoblastic T cell lymphoma.

Extranodal NK/T-cell lymphoma, nasal type (ENKL), is known as angiocentric lymphoma in the REAL classification, and also as nasal-type NK lymphoma, NK/T-cell lymphoma, and polymorphic/malignant midline reticulosis. ENKL is an aggressive non-Hodgkin's type lymphoma characterized clinically by aggressive, unrelenting destruction of the midline structures of the palate and nasal fossa, and represent about 75% of all nasal lymphomas (Metgud R S et al., J. Oral Maxillofac. Pathol. 15(1):96-100, 2011).

Cutaneous T cell lymphoma (CTCL) is caused by malignant T cells that initially migrate to the skin, causing various lesions to appear. These lesions change shape as the disease progresses, typically beginning as what appears to be a rash which can be very itchy and eventually forming plaques and tumors before metastasizing to other parts of the body. CTCL may be divided into the following types: mycosis fungoides, pagetoid reticulosis, Sezary syndrome, granulomatous slack skin, lymphomatoid papulosis, pityriasis lichenoides chronica, pityriasis lichenoides et varioliformis acuta, CD30⁺ cutaneous T-cell lymphoma, secondary cutaneous CD30⁺ large cell lymphoma, non-mycosis fungoides CD30⁻ cutaneous large T-cell lymphoma, pleomorphic T-cell lymphoma, Lennert lymphoma, and subcutaneous T-cell lymphoma.

Anaplastic large-cell lymphoma (ALCL) is a type of non-Hodgkin lymphoma involving aberrant T-cells. The term ALCL encompasses at least 4 different clinical entities, all sharing the same name Histologically, they have in common the presence of large pleomorphic cells that express CD30 and T-cell markers. Two types of ALCL are present as systemic disease and are considered aggressive lymphomas, while the other two types present as localized disease and may progress locally.

The majority of cases, greater than 90%, contain a clonal rearrangement of the T-cell receptor. Oncogeneic potential is conferred by upregulation of a tyrosine kinase gene on chromosome 2. Several different translocations involving this gene have been identified in different cases of this lymphoma. The most common is a chromosomal translocation involving the nucleophosmin gene on chromosome 5, characterized by t(2;5)(p23;q35). This results in cytoplasmic and nuclear expression of an NPM1-ALK fusion protein. Mutagenesis and functional studies have identified a plethora of NPM1-ALK interacting molecules which ultimately lead to the activation of key pathways including RAS/Erk, PLC-γ, PI3K, and Jak/signal transducers and activators of transcription (STAT) pathways, which in turn control cell proliferation and survival and cytoskeletal rearrangements. It has been demonstrated that NPM-ALK oncogenic effects are sustained by STAT3 activation. Activation of STAT3 is associated with a specific signature, which includes several transcription factors (i.e., CEBP/β), cell cycle proteins (i.e., Cyclin D, c-myc etc.), survival/apoptosis molecules (Bcl-A2, Bcl-XL, Survivin, MCL-1) and cell adhesion and mobility proteins.

Angioimmunoblastic T-cell lymphoma (AITL, formerly known as “angioimmunoblastic lymphadenopathy with dysproteinemia”) is a mature T-cell lymphoma of blood or lymph vessel immunoblasts characterized by a polymorphous lymph node infiltrate showing a marked increase in follicular dendritic cells (FDCs) and high endothelial venules (HEVs) and systemic involvement. It is also known as immunoblastic lymphadenopathy (Lukes-Collins Classification) and AILD-type (lymphogranulomatosis X) T-cell lymphoma (Kiel Classification). Clonal T-cell receptor gene rearrangements are detected in 75% of cases, and immunoglobulin gene rearrangements are seen in 10% of cases, and these cases are believed to be due to expanded EBV-driven B-cell populations. Similarly, EBV-related sequences can be detected in most cases, usually in B-cells but occasionally in T-cells.

### Methods of Treatment Based on Presence of a Biomarker

The present invention in one aspect provides methods of treating a hematological malignancy (such as lymphoma, leukemia, and myeloma) in an individual based on the status of one or more mTOR-activating aberrations in one or more mTOR-associated genes. In some embodiments, the one or more biomarkers are selected from the group consisting of biomarkers indicative of favorable response to treatment with an mTOR inhibitor, biomarkers indicative of favorable response to treatment with an immunomodulator (such as an immunostimulator or an immune checkpoint inhibitor), biomarkers indicative of favorable response to treatment with a histone deacetylase inhibitor, biomarkers indicative of favorable response to treatment with a kinase inhibitor (such as a tyrosine kinase inhibitor), and biomarkers indicative of favorable response to treatment with a cancer vaccine.

Thus, in some embodiments, there is provided a method of treating a hematological malignancy (such as lymphoma, leukemia, and myeloma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent, wherein the individual is selected for treatment based on the individual having an mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of treating a hematological malignancy (such as lymphoma, leukemia, and myeloma) in an individual comprising: (a) assessing an mTOR-activating aberration in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of a second therapeutic agent, wherein the individual is selected for treatment based on having the mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of treating a hematological malignancy (such as lymphoma, leukemia, and myeloma) in an individual comprising: (a) assessing an mTOR-activating aberration in the individual; (b) selecting (e.g., identifying or recommending) the individual for treatment based on the individual having the mTOR-activating aberration; and (c) administering to the individual i) an effective amount of a composition comprising nanoparticles comprising an mTOR inhibitor (such as a limus drug, e.g., sirolimus or a derivative thereof) and an albumin; and ii) an effective amount of a second therapeutic agent. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of selecting (including identifying or recommending) an individual having a hematological malignancy (such as lymphoma, leukemia, and myeloma) for treatment with i) an effective amount of a composition comprising nanoparticles comprising an mTOR inhibitor (such as a limus drug, e.g., sirolimus or a derivative thereof) and an albumin; and ii) an effective amount of a second therapeutic agent, wherein the method comprises (a) assessing an mTOR-activating aberration in the individual; and (b) selecting or recommending the individual for treatment based on the individual having the mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of selecting (including identifying or recommending) and treating an individual having a hematological malignancy (such as lymphoma, leukemia, and myeloma), wherein the method comprises (a) assessing an mTOR-activating aberration in the individual; (b) selecting or recommending the individual for treatment based on the individual having the mTOR-activating aberration; and (c) administering to the individual i) an effective amount of a composition comprising an mTOR inhibitor (such as a limus drug, e.g., sirolimus or a derivative thereof) and an albumin; and ii) an effective amount of a second therapeutic agent. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

Also provided herein are methods of assessing whether an individual with a hematological malignancy (such as lymphoma, leukemia, and myeloma) is more likely to respond or less likely to respond to treatment based on the individual having an mTOR-activating aberration, wherein the treatment comprises i) an effective amount of a composition comprising an mTOR inhibitor (such as a limus drug, e.g., sirolimus or a derivative thereof) and an albumin; and ii) an effective amount of a second therapeutic agent; the method comprising assessing the mTOR-activating aberration in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of a second therapeutic agent. In some embodiments, the presence of the mTOR-activating aberration indicates that the individual is more likely to respond to the treatment, and the absence of the mTOR-activating aberration indicates that the individual is less likely to respond to the treatment. In some embodiments, the amount of the mTOR inhibitor (such as a limus drug) is determined based on the status of the mTOR-activating aberration.

In some embodiments, there are also provided methods of aiding assessment of whether an individual with a hematological malignancy (such as lymphoma, leukemia, and myeloma) will likely respond to or is suitable for treatment based on the individual having an mTOR-activating aberration, wherein the treatment comprises i) an effective amount of a composition comprising an mTOR inhibitor (such as a limus drug, e.g., sirolimus or a derivative thereof) and an albumin; and ii) an effective amount of a second therapeutic agent; the method comprising assessing the mTOR-activating aberration in the individual. In some embodiments, the presence of the mTOR-activating aberration indicates that the individual will likely be responsive to the treatment, and the absence of the mTOR-activating aberration indicates that the individual is less likely to respond to the treatment. In some embodiments, the method further comprises administering to the individual i) an effective amount of a composition comprising an mTOR inhibitor (such as a limus drug, e.g., sirolimus or a derivative thereof) and an albumin; and ii) an effective amount of a second therapeutic agent.

In some embodiments, there is provided a method of identifying an individual with a hematological malignancy (such as lymphoma, leukemia, and myeloma) likely to respond to treatment comprising i) an effective amount of a composition comprising an mTOR inhibitor (such as a limus drug, e.g., sirolimus or a derivative thereof) and an albumin; and ii) an effective amount of a second therapeutic agent; the method comprising: a) assessing an mTOR-activating aberration in the individual; and b) identifying the individual based on the individual having the mTOR-activating aberration. In some embodiments, the method further comprises administering i) an effective amount of a composition comprising an mTOR inhibitor (such as a limus drug, e.g., sirolimus or a derivative thereof) and an albumin; and ii) an effective amount of a second therapeutic agent. In some embodiments, the amount of the mTOR inhibitor (such as a limus drug, e.g., sirolimus or a derivative thereof) is determined based on the status of the mTOR-activating aberration.

Also provided herein are methods of adjusting therapy treatment of an individual with a hematological malignancy (such as lymphoma, leukemia, and myeloma) receiving i) an effective amount of a composition comprising an mTOR inhibitor (such as a limus drug, e.g., sirolimus or a derivative thereof) and an albumin; and ii) an effective amount of a second therapeutic agent; the method comprising assessing an mTOR-activating aberration in a sample isolated from the individual, and adjusting the therapy treatment based on the status of the mTOR-activating aberration. In some embodiments, the amount of the mTOR inhibitor (such as a limus drug, e.g., sirolimus or a derivative thereof) is adjusted.

Also provided herein are methods of marketing a therapy comprising i) an effective amount of a composition comprising an mTOR inhibitor (such as a limus drug, e.g., sirolimus or a derivative thereof) and an albumin; and ii) an effective amount of a second therapeutic agent for use in a hematological malignancy (such as lymphoma, leukemia, and myeloma) in an individual subpopulation, the methods comprising informing a target audience about the use of the therapy for treating the individual subpopulation characterized by the individuals of such subpopulation having a sample which has an mTOR-activating aberration.

“MTOR-activating aberration” refers to a genetic aberration, an aberrant expression level and/or an aberrant activity level of one or more mTOR-associated gene that may lead to hyperactivation of the mTOR signaling pathway. “Hyperactivate” refers to increase of an activity level of a molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration.

The mTOR-activating aberration contemplated herein may include one type of aberration in one mTOR-associated gene, more than one type (such as at least about any of 2, 3, 4, 5, 6, or more) of aberrations in one mTOR-associated gene, one type of aberration in more than one (such as at least about any of 2, 3, 4, 5, 6, or more) mTOR-associated genes, or more than one type (such as at least about any of 2, 3, 4, 5, 6, or more) of aberration in more than one (such as at least about any of 2, 3, 4, 5, 6, or more) mTOR-associated genes. Different types of mTOR-activating aberration may include, but are not limited to, genetic aberrations, aberrant expression levels (e.g. overexpression or under-expression), aberrant activity levels (e.g. high or low activity levels), and aberrant phosphorylation levels. In some embodiments, a genetic aberration comprises a change to the nucleic acid (such as DNA or RNA) or protein sequence (i.e. mutation) or an aberrant epigenetic feature associated with an mTOR-associated gene, including, but not limited to, coding, non-coding, regulatory, enhancer, silencer, promoter, intron, exon, and untranslated regions of the mTOR-associated gene. In some embodiments, the mTOR-activating aberration comprises a mutation of an mTOR-associated gene, including, but not limited to, deletion, frameshift, insertion, missense mutation, nonsense mutation, point mutation, silent mutation, splice site mutation, and translocation. In some embodiments, the mutation may be a loss of function mutation for a negative regulator of the mTOR signaling pathway or a gain of function mutation of a positive regulator of the mTOR signaling pathway. In some embodiments, the genetic aberration comprises a copy number variation of an mTOR-associated gene. In some embodiments, the copy number variation of the mTOR-associated gene is caused by structural rearrangement of the genome, including deletions, duplications, inversion, and translocations. In some embodiments, the genetic aberration comprises an aberrant epigenetic feature of an mTOR-associated gene, including, but not limited to, DNA methylation, hydroxymethylation, increased or decreased histone binding, chromatin remodeling, and the like.

The mTOR-activating aberration is determined in comparison to a control or reference, such as a reference sequence (such as a nucleic acid sequence or a protein sequence), a control expression (such as RNA or protein expression) level, a control activity (such as activation or inhibition of downstream targets) level, or a control protein phosphorylation level. The aberrant expression level or the aberrant activity level in an mTOR-associated gene may be above the control level (such as about any of 10%, 20%, 30%, 40%, 60%, 70%, 80%, 90%, 100%, 200%, 500% or more above the control level) if the mTOR-associated gene is a positive regulator (i.e. activator) of the mTOR signaling pathway, or below the control level (such as about any of 10%, 20%, 30%, 40%, 60%, 70%, 80%, 90% or more below the control level) if the mTOR-associated gene is a negative regulator (i.e. inhibitor) of the mTOR signaling pathway. In some embodiments, the control level (e.g., expression level or activity level) is the median level (e.g., expression level or activity level) of a control population. In some embodiments, the control population is a population having the same hematological malignancy (such as lymphoma, leukemia, and myeloma) as the individual being treated. In some embodiments, the control population is a healthy population that does not have the hematological malignancy (such as lymphoma, leukemia, and myeloma), and optionally with comparable demographic characteristics (e.g., gender, age, ethnicity, etc.) as the individual being treated. In some embodiments, the control level (e.g., expression level or activity level) is a level (e.g., expression level or activity level) of a healthy tissue from the same individual. A genetic aberration may be determined by comparing to a reference sequence, including epigenetic patterns of the reference sequence in a control sample. In some embodiments, the reference sequence is the sequence (DNA, RNA or protein sequence) corresponding to a fully functional allele of an mTOR-associated gene, such as an allele (e.g., the prevalent allele) of the mTOR-associated gene present in a healthy population of individuals that do not have the hematological malignancy (such as lymphoma, leukemia, and myeloma), but may optionally have similar demographic characteristics (such as gender, age, ethnicity etc.) as the individual being treated.

The “status” of an mTOR-activating aberration may refer to the presence or absence of the mTOR-activating aberration in one or more mTOR-associated genes, or the aberrant level (expression or activity level, including phosphorylation level of a protein). In some embodiments, the presence of a genetic aberration (such as a mutation or a copy number variation) in one or more mTOR-associated genes as compared to a control indicates that (a) the individual is more likely to respond to treatment or (b) the individual is selected for treatment. In some embodiments, the absence of a genetic aberration in an mTOR-associated gene, or a wild-type mTOR-associated gene compared to a control, indicates that (a) the individual is less likely to respond to treatment or (b) the individual is not selected for treatment. In some embodiments, an aberrant level (such as expression level or activity level, including phosphorylation level of a protein) of one or more mTOR-associated genes is correlated with the likelihood of the individual to respond to treatment. For example, a larger deviation of the level (such as expression level or activity level, including phosphorylation level of a protein) of one or more mTOR-associated genes in the direction of hyperactivating the mTOR signaling pathway indicates that the individual is more likely to respond to treatment. In some embodiments, a prediction model based on the level(s) (such as expression level or activity level, including phosphorylation level of a protein) of one or more mTOR-associated genes is used to predict (a) the likelihood of the individual to respond to treatment and (b) whether to select the individual for treatment. The prediction model, including, for example, coefficient for each level, may be obtained by statistical analysis, such as regression analysis, using clinical trial data.

The expression level, and/or activity level of the one or more mTOR-associated genes, and/or phosphorylation level of one or more proteins encoded by the one or more mTOR-associated genes, and/or the presence or absence of one or more genetic aberrations of the one or more mTOR-associated genes can be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits.

As used herein, “based upon” includes assessing, determining, or measuring the individual's characteristics as described herein (and preferably selecting an individual suitable for receiving treatment). When the status of an mTOR-activating aberration is “used as a basis” for selection, assessing, measuring, or determining method of treatment as described herein, the mTOR-activating aberration in one or more mTOR-associated genes is determined before and/or during treatment, and the status (including presence, absence, expression level, and/or activity level of the mTOR-activating aberration) obtained is used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

The mTOR-activating aberration in an individual can be assessed or determined by analyzing a biological sample (such as tissue or fluid) from the individual. The assessment may be based on fresh biological samples or archived biological samples. Suitable biological samples include, but are not limited to, fluid containing the hematological malignancy (e.g., blood or bone marrow fluid), tissue containing the hematological malignancy (e.g., bone marrow tissue or lymph nodes), normal tissue adjacent to the hematological malignancy, normal tissue distal to the hematological malignancy, or peripheral blood lymphocytes. In some embodiments, the biological sample is tissue containing the hematological malignancy. In some embodiments, the biological sample is fluid containing the hematological malignancy. In some embodiments, the biological sample is a biopsy containing hematological malignancy cells, such as fine needle aspiration of hematological malignancy cells or laparoscopy obtained hematological malignancy cells. In some embodiments, the biopsied cells are centrifuged into a pellet, fixed, and embedded in paraffin prior to the analysis. In some embodiments, the biopsied cells are flash frozen prior to the analysis. In some embodiments, the biological sample is a plasma sample.

In some embodiments, the sample comprises a circulating cancer cell (such as a metastatic cancer cell). In some embodiments, the sample is obtained by sorting circulating tumor cells (CTCs) from blood. In some further embodiments, the CTCs have detached from a primary tumor and circulate in a bodily fluid. In some further embodiments, the CTCs have detached from a primary tumor and circulate in the bloodstream. In some embodiments, the CTCs are an indication of metastasis.

In some embodiments, the sample is mixed with an antibody that recognizes a molecule encoded by an mTOR-associated gene (such as a protein) or fragment thereof. In some embodiments, the sample is mixed with a nucleic acid that recognizes nucleic acids associated with the mTOR-associated gene (such as DNA or RNA) or fragment thereof. In some embodiments, the sample is used for sequencing analysis, such as next-generation DNA, RNA and/or exome sequencing analysis.

The mTOR-activating aberration may be assessed before the start of the treatment, at any time during the treatment, and/or at the end of the treatment. In some embodiments, the mTOR-activating aberration is assessed from about 3 days prior to the administration of an mTOR inhibitor nanoparticle composition (such as sirolimus/albumin nanoparticle composition) to about 3 days after the administration of the mTOR inhibitor nanoparticle composition in each cycle of the administration. In some embodiments, the mTOR-activating aberration is assessed on day 1 of each cycle of administration. In some embodiments, the mTOR-activating aberration is assessed in cycle 1, cycle 2 and cycle 3. In some embodiments, the mTOR-activating aberration is further assessed every 2 cycles after cycle 3.

In some embodiments, there is provided a method of treating a hematological malignancy (such as lymphoma, leukemia, and myeloma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator, wherein the individual is selected for treatment based on the individual having at least one biomarker indicative of favorable response to treatment with an immunomodulator (hereinafter also referred to as an “immunomodulator-associated biomarker”). In some embodiments, the immunomodulator-associated biomarker comprises an aberration in a gene that affects the response to treatment of a hematological malignancy (such as lymphoma, leukemia, and myeloma) in an individual with an immunomodulator (hereinafter also referred to as an “immunomodulator-associated gene”). In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

In some embodiments, there is provided a method of treating a hematological malignancy (such as lymphoma, leukemia, and myeloma) in an individual comprising: (a) assessing at least one immunomodulator-associated biomarker in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of an immunomodulator, wherein the individual is selected for treatment based on having the at least one immunomodulator-associated biomarker. In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

In some embodiments, there is provided a method of treating a hematological malignancy (such as lymphoma, leukemia, and myeloma) in an individual comprising: (a) assessing at least one immunomodulator-associated biomarker in the individual; (b) selecting (e.g., identifying or recommending) the individual for treatment based on the individual having the at least one immunomodulator-associated biomarker; and (c) administering to the individual i) an effective amount of a composition comprising nanoparticles comprising an mTOR inhibitor (such as a limus drug, e.g., sirolimus or a derivative thereof) and an albumin; and ii) an effective amount of an immunomodulator. In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

In some embodiments, there is provided a method of selecting (including identifying or recommending) an individual having a hematological malignancy (such as lymphoma, leukemia, and myeloma) for treatment with i) an effective amount of a composition comprising nanoparticles comprising an mTOR inhibitor (such as a limus drug, e.g., sirolimus or a derivative thereof) and an albumin; and ii) an effective amount of an immunomodulator, wherein the method comprises (a) assessing at least one immunomodulator-associated biomarker in the individual; and (b) selecting or recommending the individual for treatment based on the individual having the at least one immunomodulator-associated biomarker. In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

In some embodiments, there is provided a method of selecting (including identifying or recommending) and treating an individual having a hematological malignancy (such as lymphoma, leukemia, and myeloma), wherein the method comprises (a) assessing at least one immunomodulator-associated biomarker in the individual; (b) selecting or recommending the individual for treatment based on the individual having the at least one immunomodulator-associated biomarker; and (c) administering to the individual i) an effective amount of a composition comprising an mTOR inhibitor (such as a limus drug, e.g., sirolimus or a derivative thereof) and an albumin; and ii) an effective amount of an immunomodulator. In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

Also provided herein are methods of assessing whether an individual with a hematological malignancy (such as lymphoma, leukemia, and myeloma) is more likely to respond or less likely to respond to treatment based on the individual having at least one immunomodulator-associated biomarker, wherein the treatment comprises i) an effective amount of a composition comprising an mTOR inhibitor (such as a limus drug, e.g., sirolimus or a derivative thereof) and an albumin; and ii) an effective amount of an immunomodulator; the method comprising assessing at least one immunomodulator-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of an immunomodulator. In some embodiments, the presence of the at least one immunomodulator-associated biomarker indicates that the individual is more likely to respond to the treatment, and the absence of the at least one immunomodulator-associated biomarker indicates that the individual is less likely to respond to the treatment. In some embodiments, the amount of the immunomodulator is determined based on the presence of the at least one immunomodulator-associated biomarker in the individual. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

Also provided herein are methods of adjusting therapy treatment of an individual with a hematological malignancy (such as lymphoma, leukemia, and myeloma) receiving i) an effective amount of a composition comprising an mTOR inhibitor (such as a limus drug, e.g., sirolimus or a derivative thereof) and an albumin; and ii) an effective amount of an immunomodulator, the method comprising assessing at least one immunomodulator-associated biomarker in a sample isolated from the individual, and adjusting the therapy treatment based on the individual having the at least one immunomodulator-associated biomarker. In some embodiments, the amount of the immunomodulator is adjusted.

In some embodiments, there is provided a method of treating a hematological malignancy (such as lymphoma, leukemia, and myeloma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (HDACi), wherein the individual is selected for treatment based on the individual having at least one biomarker indicative of favorable response to treatment with a histone deacetylase inhibitor (hereinafter also referred to as an “HDACi-associated biomarker”). In some embodiments, the histone deacetylase inhibitor-associated biomarker comprises an aberration in a gene that affects the response to treatment of a hematological malignancy (such as lymphoma, leukemia, and myeloma) in an individual with a histone deacetylase inhibitor (hereinafter also referred to as an “HDACi-associated gene”). In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

In some embodiments, there is provided a method of treating a hematological malignancy (such as lymphoma, leukemia, and myeloma) in an individual comprising: (a) assessing at least one HDACi-associated biomarker in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of a histone deacetylase inhibitor, wherein the individual is selected for treatment based on having the at least one HDACi-associated biomarker. In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

In some embodiments, there is provided a method of treating a hematological malignancy (such as lymphoma, leukemia, and myeloma) in an individual comprising: (a) assessing at least one HDACi-associated biomarker in the individual; (b) selecting (e.g., identifying or recommending) the individual for treatment based on the individual having the at least one HDACi-associated biomarker; and (c) administering to the individual i) an effective amount of a composition comprising nanoparticles comprising an mTOR inhibitor (such as a limus drug, e.g., sirolimus or a derivative thereof) and an albumin; and ii) an effective amount of a histone deacetylase inhibitor. In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

In some embodiments, there is provided a method of selecting (including identifying or recommending) an individual having a hematological malignancy (such as lymphoma, leukemia, and myeloma) for treatment with i) an effective amount of a composition comprising nanoparticles comprising an mTOR inhibitor (such as a limus drug, e.g., sirolimus or a derivative thereof) and an albumin; and ii) an effective amount of a histone deacetylase inhibitor, wherein the method comprises (a) assessing at least one HDACi-associated biomarker in the individual; and (b) selecting or recommending the individual for treatment based on the individual having the at least one HDACi-associated biomarker. In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

In some embodiments, there is provided a method of selecting (including identifying or recommending) and treating an individual having a hematological malignancy (such as lymphoma, leukemia, and myeloma), wherein the method comprises (a) assessing at least one HDACi-associated biomarker in the individual; (b) selecting or recommending the individual for treatment based on the individual having the at least one HDACi-associated biomarker; and (c) administering to the individual i) an effective amount of a composition comprising an mTOR inhibitor (such as a limus drug, e.g., sirolimus or a derivative thereof) and an albumin; and ii) an effective amount of a histone deacetylase inhibitor. In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

Also provided herein are methods of assessing whether an individual with a hematological malignancy (such as lymphoma, leukemia, and myeloma) is more likely to respond or less likely to respond to treatment with i) an effective amount of a composition comprising an mTOR inhibitor (such as a limus drug, e.g., sirolimus or a derivative thereof) and an albumin; and ii) an effective amount of a histone deacetylase inhibitor, the method comprising assessing the at least one HDACi-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of an HDACi. In some embodiments, the presence of the at least one HDACi-associated biomarker indicates that the individual is more likely to respond to the treatment, and the absence of the at least one HDACi-associated biomarker indicates that the individual is less likely to respond to the treatment. In some embodiments, the amount of the HDACi is determined based on the presence of the at least one HDACi-associated biomarker in the individual. In some embodiments, the histone deacetylase inhibitor is selected from the group consisting of romidepsin, panobinostat, ricolinostat, and belinostat.

Also provided herein are methods of adjusting therapy treatment of an individual with a hematological malignancy (such as lymphoma, leukemia, and myeloma) receiving i) an effective amount of a composition comprising an mTOR inhibitor (such as a limus drug, e.g., sirolimus or a derivative thereof) and an albumin; and ii) an effective amount of an HDACi, the method comprising assessing at least one HDACi-associated biomarker in a sample isolated from the individual, and adjusting the therapy treatment based on the individual having the at least one HDACi-associated biomarker. In some embodiments, the amount of the HDACi is adjusted.

In some embodiments, there is provided a method of treating a hematological malignancy (such as lymphoma, leukemia, and myeloma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor), wherein the individual is selected for treatment based on the individual having at least one biomarker indicative of favorable response to treatment with a kinase inhibitor (hereinafter also referred to as a “kinase inhibitor-associated biomarker”). In some embodiments, the kinase inhibitor-associated biomarker comprises an aberration in a gene that affects the response to treatment of a hematological malignancy (such as lymphoma, leukemia, and myeloma) in an individual with a kinase inhibitor (hereinafter also referred to as a “kinase inhibitor-associated gene”). In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

In some embodiments, there is provided a method of treating a hematological malignancy (such as lymphoma, leukemia, and myeloma) in an individual comprising: (a) assessing at least one kinase inhibitor-associated biomarker in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of a kinase inhibitor (such as a tyrosine kinase inhibitor), wherein the individual is selected for treatment based on having the at least one kinase inhibitor-associated biomarker. In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

In some embodiments, there is provided a method of treating a hematological malignancy (such as lymphoma, leukemia, and myeloma) in an individual comprising: (a) assessing at least one kinase inhibitor-associated biomarker in the individual; (b) selecting (e.g., identifying or recommending) the individual for treatment based on the individual having the at least one kinase inhibitor-associated biomarker; and (c) administering to the individual i) an effective amount of a composition comprising nanoparticles comprising an mTOR inhibitor (such as a limus drug, e.g., sirolimus or a derivative thereof) and an albumin; and ii) an effective amount of a kinase inhibitor (such as a tyrosine kinase inhibitor). In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

In some embodiments, there is provided a method of selecting (including identifying or recommending) an individual having a hematological malignancy (such as lymphoma, leukemia, and myeloma) for treatment with i) an effective amount of a composition comprising nanoparticles comprising an mTOR inhibitor (such as a limus drug, e.g., sirolimus or a derivative thereof) and an albumin; and ii) an effective amount of a kinase inhibitor (such as a tyrosine kinase inhibitor), wherein the method comprises (a) assessing at least one kinase inhibitor-associated biomarker in the individual; and (b) selecting or recommending the individual for treatment based on the individual having the at least one kinase inhibitor-associated biomarker. In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

In some embodiments, there is provided a method of selecting (including identifying or recommending) and treating an individual having a hematological malignancy (such as lymphoma, leukemia, and myeloma), wherein the method comprises (a) assessing at least one kinase inhibitor-associated biomarker in the individual; (b) selecting or recommending the individual for treatment based on the individual having the at least one kinase inhibitor-associated biomarker; and (c) administering to the individual i) an effective amount of a composition comprising an mTOR inhibitor (such as a limus drug, e.g., sirolimus or a derivative thereof) and an albumin; and ii) an effective amount of a kinase inhibitor (such as a tyrosine kinase inhibitor). In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

Also provided herein are methods of assessing whether an individual with a hematological malignancy (such as lymphoma, leukemia, and myeloma) is more likely to respond or less likely to respond to treatment with i) an effective amount of a composition comprising an mTOR inhibitor (such as a limus drug, e.g., sirolimus or a derivative thereof) and an albumin; and ii) an effective amount of a kinase inhibitor (such as a tyrosine kinase inhibitor), the method comprising assessing the at least one kinase inhibitor-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of a kinase inhibitor. In some embodiments, the presence of the at least one kinase inhibitor-associated biomarker indicates that the individual is more likely to respond to the treatment, and the absence of the at least one kinase inhibitor-associated biomarker indicates that the individual is less likely to respond to the treatment. In some embodiments, the amount of the kinase inhibitor is determined based on the presence of the at least one kinase inhibitor-associated biomarker in the individual. In some embodiments, the kinase inhibitor is selected from the group consisting of nilotinib and sorafenib.

Also provided herein are methods of adjusting therapy treatment of an individual with a hematological malignancy (such as lymphoma, leukemia, and myeloma) receiving i) an effective amount of a composition comprising an mTOR inhibitor (such as a limus drug, e.g., sirolimus or a derivative thereof) and an albumin; and ii) an effective amount of a kinase inhibitor, the method comprising assessing at least one kinase inhibitor-associated biomarker in a sample isolated from the individual, and adjusting the therapy treatment based on the individual having the at least one kinase inhibitor-associated biomarker. In some embodiments, the amount of the kinase inhibitor is adjusted.

In some embodiments, there is provided a method of treating a hematological malignancy (such as lymphoma, leukemia, and myeloma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine, wherein the individual is selected for treatment based on the individual having at least one biomarker indicative of favorable response to treatment with the cancer vaccine (hereinafter also referred to as a “cancer vaccine-associated biomarker”). In some embodiments, the cancer vaccine-associated biomarker comprises an aberration in a gene that affects the response to treatment of a hematological malignancy (such as lymphoma, leukemia, and myeloma) in an individual with the cancer vaccine (such as a gene encoding an antigen used in the preparation of the cancer vaccine, also referred to herein as a “cancer vaccine-associate gene”). In some embodiments, the at least one cancer vaccine-associated biomarker comprises a mutation of a cancer vaccine-associated gene, such as a mutation that results in a neo-antigen. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

In some embodiments, there is provided a method of treating a hematological malignancy (such as lymphoma, leukemia, and myeloma) in an individual comprising: (a) assessing at least one cancer vaccine-associated biomarker in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of a cancer vaccine, wherein the individual is selected for treatment based on having the at least one cancer vaccine-associated biomarker. In some embodiments, the at least one cancer vaccine-associated biomarker comprises a mutation of a cancer vaccine-associated gene, such as a mutation that results in a neo-antigen. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

In some embodiments, there is provided a method of treating a hematological malignancy (such as lymphoma, leukemia, and myeloma) in an individual comprising: (a) assessing at least one cancer vaccine-associated biomarker in the individual; (b) selecting (e.g., identifying or recommending) the individual for treatment based on the individual having the at least one cancer vaccine-associated biomarker; and (c) administering to the individual i) an effective amount of a composition comprising nanoparticles comprising an mTOR inhibitor (such as a limus drug, e.g., sirolimus or a derivative thereof) and an albumin; and ii) an effective amount of a cancer vaccine. In some embodiments, the at least one cancer vaccine-associated biomarker comprises a mutation of a cancer vaccine-associated gene. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

In some embodiments, there is provided a method of selecting (including identifying or recommending) an individual having a hematological malignancy (such as lymphoma, leukemia, and myeloma) for treatment with i) an effective amount of a composition comprising nanoparticles comprising an mTOR inhibitor (such as a limus drug, e.g., sirolimus or a derivative thereof) and an albumin; and ii) an effective amount of a cancer vaccine, wherein the method comprises (a) assessing at least one cancer vaccine-associated biomarker in the individual; and (b) selecting or recommending the individual for treatment based on the individual having the at least one cancer vaccine-associated biomarker. In some embodiments, the at least one cancer vaccine-associated biomarker comprises a mutation of a cancer vaccine-associated gene. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

In some embodiments, there is provided a method of selecting (including identifying or recommending) and treating an individual having a hematological malignancy (such as lymphoma, leukemia, and myeloma), wherein the method comprises (a) assessing at least one cancer vaccine-associated biomarker in the individual; (b) selecting or recommending the individual for treatment based on the individual having the at least one cancer vaccine-associated biomarker; and (c) administering to the individual i) an effective amount of a composition comprising an mTOR inhibitor (such as a limus drug, e.g., sirolimus or a derivative thereof) and an albumin; and ii) an effective amount of a cancer vaccine. In some embodiments, the at least one cancer vaccine-associated biomarker comprises a mutation of a cancer vaccine-associated gene. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

Also provided herein are methods of assessing whether an individual with a hematological malignancy (such as lymphoma, leukemia, and myeloma) is more likely to respond or less likely to respond to treatment with i) an effective amount of a composition comprising an mTOR inhibitor (such as a limus drug, e.g., sirolimus or a derivative thereof) and an albumin; and ii) an effective amount of a cancer vaccine, the method comprising assessing the at least one cancer vaccine-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of a cancer vaccine. In some embodiments, the presence of the at least one cancer vaccine-associated biomarker indicates that the individual is more likely to respond to the treatment, and the absence of the at least one cancer vaccine-associated biomarker indicates that the individual is less likely to respond to the treatment. In some embodiments, the amount of the cancer vaccine is determined based on the presence of the at least one cancer vaccine-associated biomarker in the individual. In some embodiments, the cancer vaccine is selected from the group consisting of nilotinib and sorafenib.

Also provided herein are methods of adjusting therapy treatment of an individual with a hematological malignancy (such as lymphoma, leukemia, and myeloma) receiving i) an effective amount of a composition comprising an mTOR inhibitor (such as a limus drug, e.g., sirolimus or a derivative thereof) and an albumin; and ii) an effective amount of a cancer vaccine, the method comprising assessing at least one cancer vaccine-associated biomarker in a sample isolated from the individual, and adjusting the therapy treatment based on the individual having the at least one cancer vaccine-associated biomarker. In some embodiments, the amount of the cancer vaccine is adjusted.

Further contemplated are combinations of the methods described in this section, such that treatment of an individual may depend on the presence of an mTOR-activating aberration and any of the immunomodulator-, HDACi-, kinase inhibitor-, or cancer vaccine-associated biomarkers described herein.

mTOR-Activating Aberrations

The present application contemplates mTOR-activating aberrations in any one or more mTOR-associated genes described above, including deviations from the reference sequences (i.e. genetic aberrations), abnormal expression levels and/or abnormal activity levels of the one or more mTOR-associated genes. The present application encompasses treatments and methods based on the status of any one or more of the mTOR-activating aberrations disclosed herein.

The mTOR-activating aberrations described herein are associated with an increased (i.e. hyperactivated) mTOR signaling level or activity level. The mTOR signaling level or mTOR activity level described in the present application may include mTOR signaling in response to any one or any combination of the upstream signals described above, and may include mTOR signaling through mTORC1 and/or mTORC2, which may lead to measurable changes in any one or combinations of downstream molecular, cellular or physiological processes (such as protein synthesis, autophagy, metabolism, cell cycle arrest, apoptosis etc.). In some embodiments, the mTOR-activating aberration hyperactivates the mTOR activity by at least about any one of 10%, 20%, 30%, 40%, 60%, 70%, 80%, 90%, 100%, 200%, 500% or more above the level of mTOR activity without the mTOR-activating aberration. In some embodiments, the hyperactivated mTOR activity is mediated by mTORC1 only. In some embodiments, the hyperactivated mTOR activity is mediated by mTORC2 only. In some embodiments, the hyperactivated mTOR activity is mediated by both mTORC1 and mTORC2.

Methods of determining mTOR activity are known in the art. See, for example, Brian C G et al., Cancer Discovery, 2014, 4:554-563. The mTOR activity may be measured by quantifying any one of the downstream outputs (e.g. at the molecular, cellular, and/or physiological level) of the mTOR signaling pathway as described above. For example, the mTOR activity through mTORC1 may be measured by determining the level of phosphorylated 4EBP1 (e.g. P-S65-4EBP1), and/or the level of phosphorylated S6K1 (e.g. P-T389-S6K1), and/or the level of phosphorylated AKT1 (e.g. P-5473-AKT1). The mTOR activity through mTORC2 may be measured by determining the level of phosphorylated FoxO1 and/or FoxO3a. The level of a phosphorylated protein may be determined using any method known in the art, such as Western blot assays using antibodies that specifically recognize the phosphorylated protein of interest.

Candidate mTOR-activating aberrations may be identified through a variety of methods, for example, by literature search or by experimental methods known in the art, including, but not limited to, gene expression profiling experiments (e.g. RNA sequencing or microarray experiments), quantitative proteomics experiments, and gene sequencing experiments. For example, gene expression profiling experiments and quantitative proteomics experiments conducted on a sample collected from an individual having a hematological malignancy (such as lymphoma, leukemia, and myeloma) compared to a control sample may provide a list of genes and gene products (such as RNA, protein, and phosphorylated protein) that are present at aberrant levels. In some instances, gene sequencing (such as exome sequencing) experiments conducted on a sample collected from an individual having a hematological malignancy (such as lymphoma, leukemia, and myeloma) compared to a control sample may provide a list of genetic aberrations. Statistical association studies (such as genome-wide association studies) may be performed on experimental data collected from a population of individuals having a hematological malignancy to associate aberrations (such as aberrant levels or genetic aberrations) identified in the experiments with hematological malignancy. In some embodiments, targeted sequencing experiments (such as the ONCOPANEL™ test) are conducted to provide a list of genetic aberrations in an individual having a hematological malignancy (such as lymphoma, leukemia, and myeloma).

The ONCOPANEL™ test can be used to survey exonic DNA sequences of cancer related genes and intronic regions for detection of genetic aberrations, including somatic mutations, copy number variations and structural rearrangements in DNA from various sources of samples (such as a tumor biopsy or blood sample), thereby providing a candidate list of genetic aberrations that may be mTOR-activating aberrations. In some embodiments, the mTOR-associated gene aberration is a genetic aberration or an aberrant level (such as expression level or activity level) in a gene selected from the ONCOPANEL™ test (CLIA certified). See, for example, Wagle N. et al. Cancer discovery 2.1 (2012): 82-93.

An exemplary version of ONCOPANEL™ test includes 300 cancer genes and 113 introns across 35 genes. The 300 genes included in the exemplary ONCOPANEL™ test are: ABL1, AKT1, AKT2, AKT3, ALK, ALOX12B, APC, AR, ARAF, ARID1A, ARID1B, ARID2, ASXL1, ATM, ATRX, AURKA, AURKB, AXL, B2M, BAP1, BCL2, BCL2L1, BCL2L12, BCL6, BCOR, BCORL1, BLM, BMPR1A, BRAF, BRCA1, BRCA2, BRD4, BRIP1, BUB1B, CADM2, CARD11, CBL, CBLB, CCND1, CCND2, CCND3, CCNE1, CD274, CD58, CD79B, CDC73, CDH1, CDK1, CDK2, CDK4, CDK5, CDK6, CDK9, CDKN1A, CDKN1B, CDKN1C, CDKN2A, CDKN2B, CDKN2C, CEBPA, CHEK2, CIITA, CREBBP, CRKL, CRLF2, CRTC1, CRTC2, CSF1R, CSF3R, CTNNB1, CUX1, CYLD, DDB2, DDR2, DEPDCS, DICER1, DIS3, DMD, DNMT3A, EED, EGFR, EP300, EPHA3, EPHA5, EPHA7, ERBB2, ERBB3, ERBB4, ERCC2, ERCC3, ERCC4, ERCC5, ESR1, ETV1, ETV4, ETV5, ETV6, EWSR1, EXT1, EXT2, EZH2, FAM46C, FANCA, FANCC, FANCD2, FANCE, FANCF, FANCG, FAS, FBXW7, FGFR1, FGFR2, FGFR3, FGFR4, FH, FKBP9, FLCN, FLT1, FLT3, FLT4, FUS, GATA3, GATA4, GATA6, GLI1, GLI2, GLI3, GNA11, GNAQ, GNAS, GNB2L1, GPC3, GSTM5, H3F3A, HNF1A, HRAS, ID3, IDH1, IDH2, IGF1R, IKZF1, IKZF3, INSIG1, JAK2, JAK3, KCNIP1, KDM5C, KDM6A, KDM6B, KDR, KEAP1, KIT, KRAS, LINC00894, LMO1, LMO2, LMO3, MAP2K1, MAP2K4, MAP3K1, MAPK1, MCL1, MDM2, MDM4, MECOM, MEF2B, MEN1, MET, MITF, MLH1, MLL (KMT2A), MLL2 (KTM2D), MPL, MSH2, MSH6, MTOR, MUTYH, MYB, MYBL1, MYC, MYCL1 (MYCL), MYCN, MYD88, NBN, NEGR1, NF1, NF2, NFE2L2, NFKBIA, NFKBIZ, NKX2-1, NOTCH1, NOTCH2, NPM1, NPRL2, NPRL3, NRAS, NTRK1, NTRK2, NTRK3, PALB2, PARK2, PAX5, PBRM1, PDCD1LG2, PDGFRA, PDGFRB, PHF6, PHOX2B, PIK3C2B, PIK3CA, PIK3R1, PIM1, PMS1, PMS2, PNRC1, PRAME, PRDM1, PRF1, PRKAR1A, PRKCI, PRKCZ, PRKDC, PRPF40B, PRPF8, PSMD13, PTCH1, PTEN, PTK2, PTPN11, PTPRD, QKI, RAD21, RAF1, RARA, RB1, RBL2, RECQL4, REL, RET, RFWD2, RHEB, RHPN2, ROS1, RPL26, RUNX1, SBDS, SDHA, SDHAF2, SDHB, SDHC, SDHD, SETBP1, SETD2, SF1, SF3B1, SH2B3, SLITRK6, SMAD2, SMAD4, SMARCA4, SMARCB1, SMC1A, SMC3, SMO, SOCS1, SOX2, SOX9, SQSTM1, SRC, SRSF2, STAG1, STAG2, STAT3, STATE, STK11, SUFU, SUZ12, SYK, TCF3, TCF7L1, TCF7L2, TERC, TERT, TET2, TLR4, TNFAIP3, TP53, TSC1, TSC2, U2AF1, VHL, WRN, WT1, XPA, XPC, XPO1, ZNF217, ZNF708, ZRSR2. The intronic regions surveyed in the exemplary ONCOPANEL™ test are tiled on specific introns of ABL1, AKT3, ALK, BCL2, BCL6, BRAF, CIITA, EGFR, ERG, ETV1, EWSR1, FGFR1, FGFR2, FGFR3, FUS, IGH, IGL, JAK2, MLL, MYC, NPM1, NTRK1, PAX5, PDGFRA, PDGFRB, PPARG, RAF1, RARA, RET, ROS1, SS18, TRA, TRB, TRG, TMPRSS2. mTOR-activating aberrations (such as genetic aberration and aberrant levels) of any of the genes included in any embodiment or version of the ONCOPANEL™ test, including, but not limited to the genes and intronic regions listed above, are contemplated by the present application to serve as a basis for selecting an individual for treatment with the mTOR inhibitor nanoparticle compositions.

Whether a candidate genetic aberration or aberrant level is an mTOR-activating aberration can be determined with methods known in the art. Genetic experiments in cells (such as cell lines) or animal models may be performed to ascertain that the hematological malignancy-associated aberrations identified from all aberrations observed in the experiments are mTOR-activating aberrations. For example, a genetic aberration may be cloned and engineered in a cell line or animal model, and the mTOR activity of the engineered cell line or animal model may be measured and compared with corresponding cell line or animal model that do not have the genetic aberration. An increase in the mTOR activity in such experiment may indicate that the genetic aberration is a candidate mTOR-activating aberration, which may be tested in a clinical study.

### Genetic Aberrations

Genetic aberrations of one or more mTOR-associated genes may comprise a change to the nucleic acid (such as DNA and RNA) or protein sequence (i.e. mutation) or an epigenetic feature associated with an mTOR-associated gene, including, but not limited to, coding, non-coding, regulatory, enhancer, silencer, promoter, intron, exon, and untranslated regions of the mTOR-associated gene.

The genetic aberration may be a germline mutation (including chromosomal rearrangement), or a somatic mutation (including chromosomal rearrangement). In some embodiments, the genetic aberration is present in all tissues, including normal tissue and the hematological malignancy tissue, of the individual. In some embodiments, the genetic aberration is present only in the hematological malignancy tissue (such as tumor tissue, or abnormally proliferative cells in pulmonary hypertension or restenosis) of the individual. In some embodiments, the genetic aberration is present only in a fraction of the hematological malignancy tissue.

In some embodiments, the mTOR-activating aberration comprises a mutation of an mTOR-associated gene, including, but not limited to, deletion, frameshift, insertion, indel, missense mutation, nonsense mutation, point mutation, single nucleotide variation (SNV), silent mutation, splice site mutation, splice variant, and translocation. In some embodiments, the mutation may be a loss of function mutation for a negative regulator of the mTOR signaling pathway or a gain of function mutation of a positive regulator of the mTOR signaling pathway.

In some embodiments, the genetic aberration comprises a copy number variation of an mTOR-associated gene. Normally, there are two copies of each mTOR-associated gene per genome. In some embodiments, the copy number of the mTOR-associated gene is amplified by the genetic aberration, resulting in at least about any of 3, 4, 5, 6, 7, 8, or more copies of the mTOR-associated gene in the genome. In some embodiments, the genetic aberration of the mTOR-associated gene results in loss of one or both copies of the mTOR-associated gene in the genome. In some embodiments, the copy number variation of the mTOR-associated gene is loss of heterozygosity of the mTOR-associated gene. In some embodiments, the copy number variation of the mTOR-associated gene is deletion of the mTOR-associated gene. In some embodiments, the copy number variation of the mTOR-associated gene is caused by structural rearrangement of the genome, including deletions, duplications, inversion, and translocation of a chromosome or a fragment thereof.

In some embodiments, the genetic aberration comprises an aberrant epigenetic feature associated with an mTOR-associated gene, including, but not limited to, DNA methylation, hydroxymethylation, aberrant histone binding, chromatin remodeling, and the like. In some embodiments, the promotor of the mTOR-associated gene is hypermethylated in the individual, for example by at least about any of 10%, 20%, 30%, 40%, 50%, 60%, 70%, 80%, 90%, or more compared to a control level (such as a clinically accepted normal level in a standardized test).

In some embodiments, the mTOR-activating aberration is a genetic aberration (such as a mutation or a copy number variation) in any one of the mTOR-associated genes described above. In some embodiments, the mTOR-activating aberration is a mutation or a copy number variation in one or more genes selected from AKT1, MTOR, PIK3CA, PIK3CG, TSC1, TSC2, RHEB, STK11, NF1, NF2, PTEN, TP53, FGFR4, and BAP1.

Genetic aberrations in mTOR-associated genes have been identified in various human cancers, including hereditary cancers and sporadic cancers. For example, germline inactivating mutations in TSC1/2 cause tuberous sclerosis, and patients with this condition are present with lesions that include skin and brain hamartomas, renal angiomyolipomas, and renal cell carcinoma (RCC) (Krymskaya V P et al. 2011 FASEB Journal 25(6): 1922-1933). PTEN hamartoma tumor syndrome (PHTS) is linked to inactivating germline PTEN mutations and is associated with a spectrum of clinical manifestations, including breast cancer, endometrial cancer, follicular thyroid cancer, hamartomas, and RCC (Legendre C. et al. 2003 Transplantation proceedings 35(3 Suppl): 151S-153S). In addition, sporadic kidney cancer has also been shown to harbor somatic mutations in several genes in the PI3K-Akt-mTOR pathway (e.g. AKT1, MTOR, PIK3CA, PTEN, RHEB, TSC1, TSC2) (Power L A, 1990 Am. J. Hosp. Pharm. 475.5: 1033-1049; Badesch D B et al. 2010 Chest 137(2): 376-3871; Kim J C & Steinberg G D, 2001, The Journal of urology, 165(3): 745-756; McKiernan J. et al. 2010, J. Urol. 183(Suppl 4)). Of the top 50 significantly mutated genes identified by the Cancer Genome Atlas in clear cell renal cell carcinoma, the mutation rate is about 17% for gene mutations that converge on mTORC1 activation (Cancer Genome Atlas Research Network. “Comprehensive molecular characterization of clear cell renal cell carcinoma.” 2013 Nature 499: 43-49). Genetic aberrations in mTOR-associated genes have been found to confer sensitivity in individuals having cancer to treatment with a limus drug. See, for example, Wagle et al., N. Eng. J. Med. 2014, 371:1426-33; Iyer et al., Science 2012, 338: 221; Wagle et al. Cancer Discovery 2014, 4:546-553; Grabiner et al., Cancer Discovery 2014, 4:554-563; Dickson et al. Int J. Cancer 2013, 132(7): 1711-1717, and Lim et al, J Clin. Oncol. 33, 2015 suppl; abstr 11010. Genetic aberrations of mTOR-associated genes described by the above references are incorporated herein. Exemplary genetic aberrations in some mTOR-associated genes are described below, and it is understood that the present application is not limited to the exemplary genetic aberrations described herein.

In some embodiments, the mTOR-activating aberration comprises a genetic aberration in MTOR. In some embodiments, the genetic aberration comprises an activating mutation of MTOR. In some embodiments, the activating mutation of MTOR is at one or more positions (such as about any one of 1, 2, 3, 4, 5, 6, or more positions) in the protein sequence of MTOR selected from the group consisting of N269, L1357, N1421, L1433, A1459, L1460, C1483, E1519, K1771, E1799, F1888, I1973, T1977, V2006, E2014, I2017, N2206, L2209, A2210, S2215, L2216, R2217, L2220, Q2223, A2226, E2419, L2431, I2500, R2505, and D2512. In some embodiments, the activating mutation of MTOR is one or more missense mutations (such as about any one of 1, 2, 3, 4, 5, 6, or more mutations) selected from the group consisting of N269S, L1357F, N1421D, L1433S, A1459P, L1460P, C1483F, C1483R, C1483W, C1483Y, E1519T, K1771R, E1799K, F1888I, F1888I L, I1973F, T1977R, T1977K, V2006I, E2014K, I2017T, N2206S, L2209V, A2210P, 52215Y, S2215F, 52215P, L2216P, R2217W, L2220F, Q2223K, A2226S, E2419K, L2431P, 12500M, R2505P, and D2512H. In some embodiments, the activating mutation of MTOR disrupts binding of MTOR with RHEB. In some embodiments, the activating mutation of MTOR disrupts binding of MTOR with DEPTOR.

In some embodiments, the mTOR-activating aberration comprises a genetic aberration in TSC1 or TSC2. In some embodiments, the genetic aberration comprises a loss of heterozygosity of TSC1 or TSC2. In some embodiments, the genetic aberration comprises a loss of function mutation in TSC1 or TSC2. In some embodiments, the loss of function mutation is a frameshift mutation or a nonsense mutation in TSC1 or TSC2. In some embodiments, the loss of function mutation is a frameshift mutation c.1907_1908del in TSC1. In some embodiments, the loss of function mutation is a splice variant of TSC1: c.1019+1G>A. In some embodiments, the loss of function mutation is the nonsense mutation c.1073G>A in TSC2, and/or p.Trp103* in TSC1. In some embodiments, the loss of function mutation comprises a missense mutation in TSC1 or in TSC2. In some embodiments, the missense mutation is in position A256 of TSC1, and/or position Y719 of TSC2. In some embodiments, the missense mutation comprises A256V in TSC1 or Y719H in TSC2.

In some embodiments, the mTOR-activating aberration comprises a genetic aberration in RHEB. In some embodiments, the genetic aberration comprises a loss of function mutation in RHEB. In some embodiments, the loss of function mutation is at one or more positions in the protein sequence of RHEB selected from Y35 and E139. In some embodiments, the loss of function mutation in RHEB is selected from Y35N, Y35C, Y35H and E139K.

In some embodiments, the mTOR-activating aberration comprises a genetic aberration in NF1. In some embodiments, the genetic aberration comprises a loss of function mutation in NF1. In some embodiments, the loss of function mutation in NF1 is a missense mutation at position D1644 in NF1. In some embodiments, the missense mutation is D1644A in NF1.

In some embodiments, the mTOR-activating aberration comprises a genetic aberration in NF2. In some embodiments, the genetic aberration comprises a loss of function mutation in NF2. In some embodiments, the loss of function mutation in NF2 is a nonsense mutation. In some embodiments, the nonsense mutation in NF2 is c.863C>G.

In some embodiments, the mTOR-activating aberration comprises a genetic aberration in PTEN. In some embodiments, the genetic aberration comprises a deletion of PTEN in the genome.

In some embodiments, the mTOR-activating aberration comprises a genetic aberration in PI3K. In some embodiments, the genetic aberration comprises a loss of function mutation in PIK3CA or PIK3CG. In some embodiments, the loss of function mutation comprises a missense mutation at a position in PIK3CA selected from the group consisting of E542, I844, and H1047. In some embodiments, the loss of function mutation comprises a missense in PIK3CA selected from the group consisting of E542K, I844V, and H1047R.

In some embodiments, the mTOR-activating aberration comprises a genetic aberration in AKT1. In some embodiments, the genetic aberration comprises an activating mutation in AKT1. In some embodiments, the activating mutation is a missense mutation in position H238 in AKT1. In some embodiments, the missense mutation is H238Y in AKT1.

In some embodiments, the mTOR-activating aberration comprises a genetic aberration in TP53. In some embodiments, the genetic aberration comprises a loss of function mutation in TP53. In some embodiments, the loss of function mutation is a frameshift mutation in TP53, such as A39fs*5.

The genetic aberrations of the mTOR-associated genes may be assessed based on a sample, such as a sample from the individual and/or reference sample. In some embodiments, the sample is a tissue sample or nucleic acids extracted from a tissue sample. In some embodiments, the sample is a cell sample (for example a CTC sample) or nucleic acids extracted from a cell sample. In some embodiments, the sample is a tumor biopsy. In some embodiments, the sample is a tumor sample or nucleic acids extracted from a tumor sample. In some embodiments, the sample is a biopsy sample or nucleic acids extracted from the biopsy sample. In some embodiments, the sample is a Formaldehyde Fixed-Paraffin Embedded (FFPE) sample or nucleic acids extracted from the FFPE sample. In some embodiments, the sample is a blood sample. In some embodiments, cell-free DNA is isolated from the blood sample. In some embodiments, the biological sample is a plasma sample or nucleic acids extracted from the plasma sample.

The genetic aberrations of the mTOR-associated gene may be determined by any method known in the art. See, for example, Dickson et al. Int. J. Cancer, 2013, 132(7): 1711-1717; Wagle N. Cancer Discovery, 2014, 4:546-553; and Cancer Genome Atlas Research Network. Nature 2013, 499: 43-49. Exemplary methods include, but are not limited to, genomic DNA sequencing, bisulfite sequencing or other DNA sequencing-based methods using Sanger sequencing or next generation sequencing platforms; polymerase chain reaction assays; in situ hybridization assays; and DNA microarrays. The epigenetic features (such as DNA methylation, histone binding, or chromatin modifications) of one or more mTOR-associated genes from a sample isolated from the individual may be compared with the epigenetic features of the one or more mTOR-associated genes from a control sample. The nucleic acid molecules extracted from the sample can be sequenced or analyzed for the presence of the mTOR-activating genetic aberrations relative to a reference sequence, such as the wildtype sequences of AKT1, FLT-3, MTOR, PIK3CA, PIK3CG, TSC1, TSC2, RHEB, STK11, NF1, NF2, TP53, FGFR4, BAP1, KRAS, NRAS and PTEN.

In some embodiments, the genetic aberration of an mTOR-associated gene is assessed using cell-free DNA sequencing methods. In some embodiments, the genetic aberration of an mTOR-associated gene is assessed using next-generation sequencing. In some embodiments, the genetic aberration of an mTOR-associated gene isolated from a blood sample is assessed using next-generation sequencing. In some embodiments, the genetic aberration of an mTOR-associated gene is assessed using exome sequencing. In some embodiments, the genetic aberration of an mTOR-associated gene is assessed using fluorescence in-situ hybridization analysis. In some embodiments, the genetic aberration of an mTOR-associated gene is assessed prior to initiation of the methods of treatment described herein. In some embodiments, the genetic aberration of an mTOR-associated gene is assessed after initiation of the methods of treatment described herein. In some embodiments, the genetic aberration of an mTOR-associated gene is assessed prior to and after initiation of the methods of treatment described herein.

### Aberrant Levels

An aberrant level of an mTOR-associated gene may refer to an aberrant expression level or an aberrant activity level.

Aberrant expression level of an mTOR-associated gene comprises an increase or decrease in the level of a molecule encoded by the mTOR-associated gene compared to the control level. The molecule encoded by the mTOR-associated gene may include RNA transcript(s) (such as mRNA), protein isoform(s), phosphorylated and/or dephosphorylated states of the protein isoform(s), ubiquitinated and/or de-ubiquitinated states of the protein isoform(s), membrane localized (e.g. myristoylated, palmitoylated, and the like) states of the protein isoform(s), other post-translationally modified states of the protein isoform(s), or any combination thereof.

Aberrant activity level of an mTOR-associated gene comprises enhancement or repression of a molecule encoded by any downstream target gene of the mTOR-associated gene, including epigenetic regulation, transcriptional regulation, translational regulation, post-translational regulation, or any combination thereof of the downstream target gene. Additionally, activity of an mTOR-associated gene comprises downstream cellular and/or physiological effects in response to the mTOR-activating aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the mTOR-activating aberration (e.g. aberrant expression level) comprises an aberrant protein phosphorylation level. In some embodiments, the aberrant phosphorylation level is in a protein encoded by an mTOR-associated gene selected from the group consisting of AKT, TSC2, mTOR, PRAS40, S6K, S6, and 4EBP1. Exemplary phosphorylated species of mTOR-associated genes that may serve as relevant biomarkers include, but are not limited to, AKT S473 phosphorylation, PRAS40 T246 phosphorylation, mTOR S2448 phosphorylation, 4EBP1 T36 phosphorylation, S6K T389 phosphorylation, 4EBP1 T70 phosphorylation, and S6 S235 phosphorylation. In some embodiments, the individual is selected for treatment if the protein in the individual is phosphorylated. In some embodiments, the individual is selected for treatment if the protein in the individual is not phosphorylated. In some embodiments, the phosphorylation status of the protein is determined by immunohistochemistry.

The levels (such as expression levels and/or activity levels) of an mTOR-associated gene in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. In some embodiments, the sample is a tissue containing the hematological malignancy, normal tissue adjacent to said hematological malignancy tissue, normal tissue distal to said hematological malignancy tissue, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing hematological malignancy cells. In a further embodiment, the biopsy is a fine needle aspiration of hematological malignancy cells. In a further embodiment, the biopsy is laparoscopy obtained hematological malignancy cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes a molecule encoded by the mTOR-associated gene. In some embodiments, a biopsy is taken to determine whether an individual has hematological malignancy and is then used as a sample. In some embodiments, the sample comprises surgically obtained hematological malignancy cells. In some embodiments, samples may be obtained at different times than when the determining of expression levels of mTOR-associated gene occurs.

In some embodiments, the sample comprises a circulating metastatic cancer cell. In some embodiments, the sample is obtained by sorting circulating tumor cells (CTCs) from blood. In a further embodiment, the CTCs have detached from a primary tumor and circulate in a bodily fluid. In yet a further embodiment, the CTCs have detached from a primary tumor and circulate in the bloodstream. In a further embodiment, the CTCs are an indication of metastasis.

In some embodiments, the level of a protein encoded by an mTOR-associated gene is determined to assess the aberrant expression level of the mTOR-associated gene. In some embodiments, the level of a protein encoded by a downstream target gene of an mTOR-associated gene is determined to assess the aberrant activity level of the mTOR-associated gene. In some embodiments, protein level is determined using one or more antibodies specific for one or more epitopes of the individual protein or proteolytic fragments thereof. Detection methodologies suitable for use in the practice of the invention include, but are not limited to, immunohistochemistry, enzyme linked immunosorbent assays (ELISAs), Western blotting, mass spectroscopy, and immuno-PCR. In some embodiments, levels of protein(s) encoded by the mTOR-associated gene and/or downstream target gene(s) thereof in a sample are normalized (such as divided) by the level of a housekeeping protein (such as glyceraldehyde 3-phosphate dehydrogenase, or GAPDH) in the same sample.

In some embodiments, the level of an mRNA encoded by an mTOR-associated gene is determined to assess the aberrant expression level of the mTOR-associated gene. In some embodiments, the level of an mRNA encoded by a downstream target gene of an mTOR-associated gene is determined to assess the aberrant activity level of the mTOR-associated gene. In some embodiments, a reverse-transcription (RT) polymerase chain reaction (PCR) assay (including a quantitative RT-PCR assay) is used to determine the mRNA levels. In some embodiments, a gene chip or next-generation sequencing methods (such as RNA (cDNA) sequencing or exome sequencing) are used to determine the levels of RNA (such as mRNA) encoded by the mTOR-associated gene and/or downstream target genes thereof. In some embodiments, an mRNA level of the mTOR-associated gene and/or downstream target genes thereof in a sample are normalized (such as divided) by the mRNA level of a housekeeping gene (such as GAPDH) in the same sample.

The levels of an mTOR-associated gene may be a high level or a low level as compared to a control or reference. In some embodiments, wherein the mTOR-associated gene is a positive regulator of the mTOR activity (such as mTORC1 and/or mTORC2 activity), the aberrant level of the mTOR associated gene is a high level compared to the control. In some embodiments, wherein the mTOR-associated gene is a negative regulator of the mTOR activity (such as mTORC1 and/or mTORC2 activity), the aberrant level of the mTOR associated gene is a low level compared to the control.

In some embodiments, the level of the mTOR-associated gene in an individual is compared to the level of the mTOR-associated gene in a control sample. In some embodiments, the level of the mTOR-associated gene in an individual is compared to the level of the mTOR-associated gene in multiple control samples. In some embodiments, multiple control samples are used to generate a statistic that is used to classify the level of the mTOR-associated gene in an individual with a hematological malignancy (such as lymphoma, leukemia, and myeloma).

The classification or ranking of the level (i.e., high or low) of the mTOR-associated gene may be determined relative to a statistical distribution of control levels. In some embodiments, the classification or ranking is relative to a control sample, such as a normal tissue (e.g. peripheral blood mononuclear cells), or a normal epithelial cell sample (e.g. a buccal swap or a skin punch) obtained from the individual. In some embodiments, the level of the mTOR-associated gene is classified or ranked relative to a statistical distribution of control levels. In some embodiments, the level of the mTOR-associated gene is classified or ranked relative to the level from a control sample obtained from the individual.

Control samples can be obtained using the same sources and methods as non-control samples. In some embodiments, the control sample is obtained from a different individual (for example an individual not having the hematological malignancy; an individual having a benign or less advanced form of a disease corresponding to the hematological malignancy; and/or an individual sharing similar ethnic, age, and gender). In some embodiments when the sample is a tumor tissue sample, the control sample may be a non-cancerous sample from the same individual. In some embodiments, multiple control samples (for example from different individuals) are used to determine a range of levels of the mTOR-associated genes in a particular tissue, organ, or cell population.

In some embodiments, the control sample is a cultured tissue or cell that has been determined to be a proper control. In some embodiments, the control is a cell that does not have the mTOR-activating aberration. In some embodiments, a clinically accepted normal level in a standardized test is used as a control level for determining the aberrant level of the mTOR-associated gene. In some embodiments, the level of the mTOR-associated gene or downstream target genes thereof in the individual is classified as high, medium or low according to a scoring system, such as an immunohistochemistry-based scoring system.

In some embodiments, the level of the mTOR-associated gene is determined by measuring the level of the mTOR-associated gene in an individual and comparing to a control or reference (e.g., the median level for the given patient population or level of a second individual). For example, if the level of the mTOR-associated gene for the single individual is determined to be above the median level of the patient population, that individual is determined to have high expression level of the mTOR-associated gene. Alternatively, if the level of the mTOR-associated gene for the single individual is determined to be below the median level of the patient population, that individual is determined to have low expression level of the mTOR-associated gene. In some embodiments, the individual is compared to a second individual and/or a patient population which is responsive to the treatment. In some embodiments, the individual is compared to a second individual and/or a patient population which is not responsive to the treatment. In some embodiments, the levels are determined by measuring the level of a nucleic acid encoded by the mTOR-associated gene and/or a downstream target gene thereof. For example, if the level of a molecule (such as an mRNA or a protein) encoded by the mTOR-associated gene for the single individual is determined to be above the median level of the patient population, that individual is determined to have a high level of the molecule (such as mRNA or protein) encoded by the mTOR-associated gene. Alternatively, if the level of a molecule (such as an mRNA or a protein) encoded by the mTOR-associated gene for the single individual is determined to be below the median level of the patient population, that individual is determined to have a low level of the molecule (such as mRNA or protein) encoded by the mTOR-associated gene.

In some embodiments, the control level of an mTOR-associated gene is determined by obtaining a statistical distribution of the levels of mTOR-associated gene. In some embodiments, the level of the mTOR-associated gene is classified or ranked relative to control levels or a statistical distribution of control levels.

In some embodiments, bioinformatics methods are used for the determination and classification of the levels of the mTOR-associated gene, including the levels of downstream target genes of the mTOR-associated gene as a measure of the activity level of the mTOR-associated gene. Numerous bioinformatics approaches have been developed to assess gene set expression profiles using gene expression profiling data. Methods include but are not limited to those described in Segal, E. et al. Nat. Genet. 34:66-176 (2003); Segal, E. et al. Nat. Genet. 36:1090-1098 (2004); Barry, W. T. et al. Bioinformatics 21:1943-1949 (2005); Tian, L. et al. Proc Nat'l Acad Sci USA 102:13544-13549 (2005); Novak B A and Jain A N. Bioinformatics 22:233-41 (2006); Maglietta R et al. Bioinformatics 23:2063-72 (2007); Bussemaker H J, BMC Bioinformatics 8 Suppl 6:S6 (2007).

In some embodiments, the control level is a pre-determined threshold level. In some embodiments, mRNA level is determined, and a low level is an mRNA level less than about any of 1, 0.9, 0.8, 0.7, 0.6, 0.5, 0.4, 0.3, 0.2, 0.1, 0.05, 0.02, 0.01, 0.005, 0.002, 0.001 or less time that of what is considered as clinically normal or of the level obtained from a control. In some embodiments, a high level is an mRNA level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times that of what is considered as clinically normal or of the level obtained from a control.

In some embodiments, protein expression level is determined, for example by Western blot or an enzyme-linked immunosorbent assay (ELISA). For example, the criteria for low or high levels can be made based on the total intensity of a band on a protein gel corresponding to the protein encoded by the mTOR-associated gene that is blotted by an antibody that specifically recognizes the protein encoded by the mTOR-associated gene, and normalized (such as divided) by a band on the same protein gel of the same sample corresponding to a housekeeping protein (such as GAPDH) that is blotted by an antibody that specifically recognizes the housekeeping protein (such as GAPDH). In some embodiments, the protein level is low if the protein level is less than about any of 1, 0.9, 0.8, 0.7, 0.6, 0.5, 0.4, 0.3, 0.2, 0.1, 0.05, 0.02, 0.01, 0.005, 0.002, 0.001 or less time of what is considered as clinically normal or of the level obtained from a control. In some embodiments, the protein level is high if the protein level is more than about any of 1.1, 1.2, 1.3, 1.5, 1.7, 2, 2.2, 2.5, 2.7, 3, 5, 7, 10, 20, 50, or 100 times or more than 100 times of what is considered as clinically normal or of the level obtained from a control.

In some embodiments, protein expression level is determined, for example by immunohistochemistry. For example, the criteria for low or high levels can be made based on the number of positive staining cells and/or the intensity of the staining, for example by using an antibody that specifically recognizes the protein encoded by the mTOR-associated gene. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than a positive control staining. In some embodiments, the level is high if more than about 40%, 45%, 50%, 55%, 60%, 65%, 70%, 75%, 80%, 85%, or 90%, cells have positive staining. In some embodiments, the level is high if the staining is as intense as positive control staining. In some embodiments, the level is high if the staining is 80%, 85%, or 90% as intense as positive control staining.

In some embodiments, the scoring is based on an “H-score” as described in US Pat. Pub. No. 2013/0005678. An H-score is obtained by the formula: 3×percentage of strongly staining cells+2×percentage of moderately staining cells+percentage of weakly staining cells, giving a range of 0 to 300.

In some embodiments, strong staining, moderate staining, and weak staining are calibrated levels of staining, wherein a range is established and the intensity of staining is binned within the range. In some embodiments, strong staining is staining above the 75th percentile of the intensity range, moderate staining is staining from the 25th to the 75th percentile of the intensity range, and low staining is staining is staining below the 25th percentile of the intensity range. In some aspects one skilled in the art, and familiar with a particular staining technique, adjusts the bin size and defines the staining categories.

In some embodiments, the label high staining is assigned where greater than 50% of the cells stained exhibited strong reactivity, the label no staining is assigned where no staining was observed in less than 50% of the cells stained, and the label low staining is assigned for all of other cases.

In some embodiments, the assessment and/or scoring of the genetic aberration or the level of the mTOR-associated gene in a sample, patient, etc., is performed by one or more experienced clinicians, i.e., those who are experienced with the mTOR-associated gene expression and the mTOR-associated gene product staining patterns. For example, in some embodiments, the clinician(s) is blinded to clinical characteristics and outcome for the samples, patients, etc. being assessed and scored.

In some embodiments, level of protein phosphorylation is determined. The phosphorylation status of a protein may be assessed from a variety of sample sources. In some embodiments, the sample is a tumor biopsy. The phosphorylation status of a protein may be assessed via a variety of methods. In some embodiments, the phosphorylation status is assessed using immunohistochemistry. The phosphorylation status of a protein may be site specific. The phosphorylation status of a protein may be compared to a control sample. In some embodiments, the phosphorylation status is assessed prior to initiation of the methods of treatment described herein. In some embodiments, the phosphorylation status is assessed after initiation of the methods of treatment described herein. In some embodiments, the phosphorylation status is assessed prior to and after initiation of the methods of treatment described herein.

Further provided herein are methods of directing treatment of a hematological malignancy (such as lymphoma, leukemia, and myeloma) by delivering a sample to a diagnostic lab for determination of the level of an mTOR-associated gene; providing a control sample with a known level of the mTOR-associated gene; providing an antibody to a molecule encoded by the mTOR-associated gene or an antibody to a molecule encoded by a downstream target gene of the mTOR-associated gene; individually contacting the sample and control sample with the antibody, and/or detecting a relative amount of antibody binding, wherein the level of the sample is used to provide a conclusion that a patient should receive a treatment with any one of the methods described herein.

Also provided herein are methods of directing treatment of a hematological malignancy (such as lymphoma, leukemia, and myeloma), further comprising reviewing or analyzing data relating to the status (such as presence/absence or level) of an mTOR-activating aberration in a sample; and providing a conclusion to an individual, such as a health care provider or a health care manager, about the likelihood or suitability of the individual to respond to a treatment, the conclusion being based on the review or analysis of data. In one aspect of the invention a conclusion is the transmission of the data over a network.

### Resistance Biomarkers

Genetic aberrations and aberrant levels of certain genes may be associated with resistance to the treatment methods described herein. In some embodiments, the individual having an aberration (such as genetic aberration or aberrant level) in a resistance biomarker is excluded from the methods of treatment using the mTOR inhibitor nanoparticles as described herein. In some embodiments, the status of the resistance biomarkers combined with the status of one or more of the mTOR-activating aberrations are used as the basis for selecting an individual for any one of the methods of treatment using mTOR inhibitor nanoparticles as described herein.

For example, TFE3, also known as transcription factor binding to IGHM enhancer 3, TFEA, RCCP2, RCCX1, or bHLHe33, is a transcription factor that specifically recognizes and binds MUE3-type E-box sequences in the promoters of genes. TFE3 promotes expression of genes downstream of transforming growth factor beta (TGF-beta) signaling. Translocation of TFE3 has been associated with renal cell carcinomas and other cancers. In some embodiments, the nucleic acid sequence of a wildtype TFE3 gene is identified by the Genbank accession number NC_000023.11 from nucleotide 49028726 to nucleotide 49043517 of the complement strand of chromosome X according to the GRCh38.p2 assembly of the human genome. Exemplary translocations of TFE3 that may be associated with resistance to treatment using the mTOR inhibitor nanoparticles as described herein include, but are not limited to, Xp11 translocation, such as t(X; 1)(p11.2; q21), t(X; 1)(p11.2; p34), (X; 17)(p11.2; q25.3), and inv(X)(p11.2; q12). Translocation of the TFE3 locus can be assessed using immunohistochemical methods or fluorescence in situ hybridization (FISH).

### Dosing and Method of Administering

The dose of the mTOR inhibitor nanoparticle composition (such as sirolimus/albumin nanoparticle composition) administered to an individual (e.g., a human) in combination therapy may vary with the particular composition, the method of administration, and the particular stage of hematological malignancy being treated. The amount should be sufficient to produce a desirable response, such as a therapeutic or prophylactic response against hematological malignancy. In some embodiments, the amount of mTOR inhibitor (such as a limus drug, e.g., sirolimus or a derivative thereof) in the composition is below the level that induces a toxicological effect (e.g., an effect above a clinically acceptable level of toxicity) or is at a level where a potential side effect can be controlled or tolerated when the mTOR inhibitor nanoparticle composition is administered to the individual.

In some embodiments, the mTOR inhibitor nanoparticle composition (such as sirolimus/albumin nanoparticle composition) is administered to the individual simultaneously with the second therapeutic agent. For example, the mTOR inhibitor nanoparticle compositions and the second therapeutic agent are administered with a time separation of no more than about 15 minutes, such as no more than about any of 10, 5, or 1 minutes. In one example, wherein the compounds are in solution, simultaneous administration can be achieved by administering a solution containing the combination of compounds. In another example, simultaneous administration of separate solutions, one of which contains the mTOR inhibitor nanoparticle composition (such as sirolimus/albumin nanoparticle composition) and the other of which contains the second therapeutic agent, can be employed. In one example, simultaneous administration can be achieved by administering a composition containing the combination of compounds. In another example, simultaneous administration can be achieved by administering two separate compositions, one comprising the mTOR inhibitor nanoparticle composition (such as sirolimus/albumin nanoparticle composition) and the other comprising the second therapeutic agent. In some embodiments, simultaneous administration of the mTOR inhibitor (such as a limus drug, e.g., sirolimus or a derivative thereof) in the nanoparticle composition and the second therapeutic agent can be combined with supplemental doses of the mTOR inhibitor and/or the second therapeutic agent.

In other embodiments, the mTOR inhibitor nanoparticle composition (such as sirolimus/albumin nanoparticle composition) and the second therapeutic agent are not administered simultaneously. In some embodiments, the mTOR inhibitor nanoparticle composition (such as sirolimus/albumin nanoparticle composition) is administered before the second therapeutic agent. In other embodiments, the second therapeutic agent is administered before the mTOR inhibitor nanoparticle composition (such as sirolimus/albumin nanoparticle composition). The time difference in non-simultaneous administrations can be greater than 1 minute, five minutes, 10 minutes, 15 minutes, 30 minutes, 45 minutes, 60 minutes, two hours, three hours, six hours, nine hours, 12 hours, 24 hours, 36 hours, or 48 hours. In other embodiments, the first administered compound is provided time to take effect on the patient before the second administered compound is administered. In some embodiments, the difference in time does not extend beyond the time for the first administered compound to complete its effect in the patient, or beyond the time the first administered compound is completely or substantially eliminated or deactivated in the patient.

In some embodiments, the administration of the mTOR inhibitor nanoparticle composition (such as sirolimus/albumin nanoparticle composition) and the second therapeutic agent are concurrent, i.e., the administration period of the mTOR inhibitor nanoparticle composition and that of the second therapeutic agent overlap with each other. In some embodiments, the mTOR inhibitor nanoparticle composition (such as sirolimus/albumin nanoparticle composition) is administered for at least one cycle (for example, at least any of 2, 3, or 4 cycles) prior to the administration of the second therapeutic agent. In some embodiments, the second therapeutic agent is administered for at least any of one, two, three, or four weeks. In some embodiments, the administrations of the mTOR inhibitor nanoparticle composition (such as sirolimus/albumin nanoparticle composition) and the second therapeutic agent are initiated at about the same time (for example, within any one of 1, 2, 3, 4, 5, 6, or 7 days). In some embodiments, the administrations of the mTOR inhibitor nanoparticle composition (such as sirolimus/albumin nanoparticle composition) and the second therapeutic agent are terminated at about the same time (for example, within any one of 1, 2, 3, 4, 5, 6, or 7 days). In some embodiments, the administration of the second therapeutic agent continues (for example for about any one of 1, 2, 3, 4, 5, 6, 7, 8, 9, 10, 11, or 12 months) after the termination of the administration of the mTOR inhibitor nanoparticle composition (such as sirolimus/albumin nanoparticle composition). In some embodiments, the administration of the second therapeutic agent is initiated after (for example after about any one of 1, 2, 3, 4, 5, 6, 7, 8, 9, 10, 11, or 12 months) the initiation of the administration of the mTOR inhibitor nanoparticle composition (such as sirolimus/albumin nanoparticle composition). In some embodiments, the administrations of the mTOR inhibitor nanoparticle composition (such as sirolimus/albumin nanoparticle composition) and the second therapeutic agent are initiated and terminated at about the same time. In some embodiments, the administrations of the mTOR inhibitor nanoparticle composition (such as sirolimus/albumin nanoparticle composition) and the second therapeutic agent are initiated at about the same time and the administration of the second therapeutic agent continues (for example for about any one of 1, 2, 3, 4, 5, 6, 7, 8, 9, 10, 11, or 12 months) after the termination of the administration of the mTOR inhibitor nanoparticle composition. In some embodiments, the administration of the mTOR inhibitor nanoparticle composition (such as sirolimus/albumin nanoparticle composition) and the second therapeutic agent stop at about the same time and the administration of the second therapeutic agent is initiated after (for example after about any one of 1, 2, 3, 4, 5, 6, 7, 8, 9, 10, 11, or 12 months) the initiation of the administration of the mTOR inhibitor nanoparticle composition.

In some embodiments, the administration of the mTOR inhibitor nanoparticle composition (such as sirolimus/albumin nanoparticle composition) and the second therapeutic agent are non-concurrent. For example, in some embodiments, the administration of the mTOR inhibitor nanoparticle composition (such as sirolimus/albumin nanoparticle composition) is terminated before the second therapeutic agent is administered. In some embodiments, the administration of the second therapeutic agent is terminated before the mTOR inhibitor nanoparticle composition (such as sirolimus/albumin nanoparticle composition) is administered. The time period between these two non-concurrent administrations can range from about two to eight weeks, such as about four weeks.

The dosing frequency of the mTOR inhibitor nanoparticle composition (such as sirolimus/albumin nanoparticle composition) and the second therapeutic agent may be adjusted over the course of the treatment, based on the judgment of the administering physician. When administered separately, the mTOR inhibitor nanoparticle composition (such as sirolimus/albumin nanoparticle composition) and the second therapeutic agent can be administered at different dosing frequency or intervals. For example, the mTOR inhibitor nanoparticle composition (such as sirolimus/albumin nanoparticle composition) can be administered weekly, while a second therapeutic agent can be administered more or less frequently. In some embodiments, sustained continuous release formulation of the nanoparticle and/or second therapeutic agent may be used. Various formulations and devices for achieving sustained release are known in the art. A combination of the administration configurations described herein can also be used.

The mTOR inhibitor nanoparticle composition (such as sirolimus/albumin nanoparticle composition) and the second therapeutic agent can be administered using the same route of administration or different routes of administration. In some embodiments (for both simultaneous and sequential administrations), the mTOR inhibitor (such as a limus drug, e.g., sirolimus or a derivative thereof) in the mTOR inhibitor nanoparticle composition and the second therapeutic agent are administered at a predetermined ratio. For example, in some embodiments, the ratio by weight of the mTOR inhibitor (such as a limus drug, e.g., sirolimus or a derivative thereof) in the mTOR inhibitor nanoparticle composition and the second therapeutic agent is about 1 to 1. In some embodiments, the weight ratio may be between about 0.001 to about 1 and about 1000 to about 1, or between about 0.01 to about 1 and 100 to about 1. In some embodiments, the ratio by weight of the mTOR inhibitor (such as a limus drug, e.g., sirolimus or a derivative thereof) in the mTOR inhibitor nanoparticle composition and the second therapeutic agent is less than about any of 100:1, 50:1, 30:1, 10:1, 9:1, 8:1, 7:1, 6:1, 5:1, 4:1, 3:1, 2:1, and 1:1 In some embodiments, the ratio by weight of the mTOR inhibitor (such as a limus drug, e.g., sirolimus or a derivative thereof) in the mTOR inhibitor nanoparticle composition and the second therapeutic agent is more than about any of 1:1, 2:1, 3:1, 4:1, 5:1, 6:1, 7:1, 8:1, 9:1, 30:1, 50:1, 100:1. Other ratios are contemplated.

The doses required for the mTOR inhibitor (such as a limus drug, e.g., sirolimus or a derivative thereof) in the mTOR inhibitor nanoparticle composition and/or the second therapeutic agent may (but not necessarily) be the same or lower than what is normally required when each agent is administered alone. Thus, in some embodiments, a subtherapeutic amount of the mTOR inhibitor (such as a limus drug, e.g., sirolimus or a derivative thereof) in the mTOR inhibitor nanoparticle composition and/or the second therapeutic agent is administered. “Subtherapeutic amount” or “subtherapeutic level” refer to an amount that is less than the therapeutic amount, that is, less than the amount normally used when the mTOR inhibitor nanoparticle composition (such as sirolimus/albumin nanoparticle composition) and/or the second therapeutic agent are administered alone. The reduction may be reflected in terms of the amount administered at a given administration and/or the amount administered over a given period of time (reduced frequency).

In some embodiments, enough second therapeutic agent is administered so as to allow reduction of the normal dose of the mTOR inhibitor (such as a limus drug, e.g., sirolimus or a derivative thereof) in the mTOR inhibitor nanoparticle composition required to effect the same degree of treatment by at least about any of 5%, 10%, 20%, 30%, 50%, 60%, 70%, 80%, 90%, or more. In some embodiments, enough mTOR inhibitor (such as a limus drug, e.g., sirolimus or a derivative thereof) in the mTOR inhibitor nanoparticle composition is administered so as to allow reduction of the normal dose of the second therapeutic agent required to effect the same degree of treatment by at least about any of 5%, 10%, 20%, 30%, 50%, 60%, 70%, 80%, 90%, or more.

In some embodiments, the dose of both the mTOR inhibitor (such as a limus drug, e.g., sirolimus or a derivative thereof) in the mTOR inhibitor nanoparticle composition and the second therapeutic agent are reduced as compared to the corresponding normal dose of each when administered alone. In some embodiments, both the mTOR inhibitor (such as a limus drug, e.g., sirolimus or a derivative thereof) in the mTOR inhibitor nanoparticle composition and the second therapeutic agent are administered at a subtherapeutic, i.e., reduced, level. In some embodiments, the dose of the mTOR inhibitor (such as a limus drug, e.g., sirolimus or a derivative thereof) in the mTOR inhibitor nanoparticle composition and/or the second therapeutic agent is substantially less than the established maximum toxic dose (MTD). For example, the dose of the mTOR inhibitor nanoparticle composition (such as sirolimus/albumin nanoparticle composition) and/or the second therapeutic agent is less than about 50%, 40%, 30%, 20%, or 10% of the MTD.

A combination of the administration configurations described herein can be used. The combination therapy methods described herein may be performed alone or in conjunction with another therapy, such as surgery, radiation, gene therapy, immunotherapy, bone marrow transplantation, stem cell transplantation, hormone therapy, targeted therapy, cryotherapy, ultrasound therapy, photodynamic therapy, and/or chemotherapy and the like. Additionally, a person having a greater risk of developing the hematological malignancy may receive treatments to inhibit and/or delay the development of the disease.

As will be understood by those of ordinary skill in the art, the appropriate doses of second chemotherapeutic agents will be approximately those already employed in clinical therapies wherein the second therapeutic agent is administered alone or in combination with other chemotherapeutic agents. Variation in dosage will likely occur depending on the condition being treated. As described above, in some embodiments, the second chemotherapeutic agent may be administered at a reduced level.

Thus, in some embodiments, according to any of the methods described herein where the second therapeutic agent is pomalidomide, the pomalidomide is administered as a daily oral dose of about 1 to about 4 mg (including for example about any of 1, 1.5, 2, 2.5, 3, 3.5, or 4 mg, including any range between these values) on days 1-21 of a 28-day cycle for at least one (such as at least any of 2, 3, 4, 5, 6, 7, 8, 9, 10 or more) cycle. In some embodiments, the pomalidomide is administered as a daily oral dose of no more than about 4 (such as no more than about any of 4, 3.5, 3, 2.5, 2, 1.5, 1 or less) mg on days 1-21 of a 28-day cycle for at least one (such as at least any of 2, 3, 4, 5, 6, 7, 8, 9, 10 or more) cycle. In some embodiments, the pomalidomide is administered as a daily oral dose of about 4 mg on days 1-21 of a 28-day cycle for at least one (such as at least any of 2, 3, 4, 5, 6, 7, 8, 9, 10 or more) cycle. In some embodiments, the pomalidomide is administered until progression of the hematological malignancy. In some embodiments, the method further comprises administering dexamethasone to the individual. In some embodiments, the dexamethasone is administered as a daily dose (such as an oral dose) of about 20 to about 40 mg (including for example about any of 20, 25, 30, 35, or 40 mg, including any range between these values) on days 1, 8, 15, and 22 of a 28-day cycle for at least one (such as at least any of 2, 3, 4, 5, 6, 7, 8, 9, 10 or more) cycle. In some embodiments, the dexamethasone is administered as a daily dose (such as an oral dose) of about 40 mg on days 1, 8, 15, and 22 of a 28-day cycle for at least one (such as at least any of 2, 3, 4, 5, 6, 7, 8, 9, 10 or more) cycle. The dose of pomalidomide may be discontinued or interrupted, with or without dose reduction, to manage adverse drug reactions. In some embodiments, the pomalidomide is administered according to the prescribing information of an approved brand of pomalidomide.

In some embodiments, according to any of the methods described herein where the second therapeutic agent is lenalidomide, the lenalidomide is administered as a daily oral dose of about 15 to about 25 mg (including for example about any of 15, 16, 17, 18, 19, 20, 21, 22, 23, 24, or 25 mg, including any range between these values) on days 1-21 of a 28-day cycle for at least one (such as at least any of 2, 3, 4, 5, 6, 7, 8, 9, 10 or more) cycle. In some embodiments, the lenalidomide is administered as a daily oral dose of no more than about 25 (such as no more than about any of 25, 22.5, 20, 17.5, 15, 12.5, 10, or less) mg on days 1-21 of a 28-day cycle for at least one (such as at least any of 2, 3, 4, 5, 6, 7, 8, 9, 10 or more) cycle. In some embodiments, the lenalidomide is administered as a daily oral dose of about 25 mg on days 1-21 of a 28-day cycle for at least one (such as at least any of 2, 3, 4, 5, 6, 7, 8, 9, 10 or more) cycle. In some embodiments, the lenalidomide is administered until progression of the hematological malignancy. In some embodiments, the method further comprises administering dexamethasone to the individual. In some embodiments, the dexamethasone is administered as a daily dose (such as an oral dose) of about 20 to about 40 mg (including for example about any of 20, 25, 30, 35, or 40 mg, including any range between these values) on days 1, 8, 15, and 22 of a 28-day cycle for at least one (such as at least any of 2, 3, 4, 5, 6, 7, 8, 9, 10 or more) cycle. In some embodiments, the dexamethasone is administered as a daily dose (such as an oral dose) of about 40 mg on days 1, 8, 15, and 22 of a 28-day cycle for at least one (such as at least any of 2, 3, 4, 5, 6, 7, 8, 9, 10 or more) cycle. The dose of lenalidomide may be discontinued or interrupted, with or without dose reduction, to manage adverse drug reactions. In some embodiments, the lenalidomide is administered according to the prescribing information of an approved brand of lenalidomide.

In some embodiments, according to any of the methods described herein where the second therapeutic agent is romidepsin, the romidepsin is administered as an IV dose of about 5 to about 14 mg/m² (including for example about any of 5, 6, 7, 8, 9, 10, 11, 12, 13, or 14 mg/m², including any range between these values) on days 1, 8, and 15 of a 28-day cycle for at least one (such as at least any of 2, 3, 4, 5, 6, 7, 8, 9, 10 or more) cycle. In some embodiments, the romidepsin is administered as an IV dose of no more than about 14 (such as no more than about any of 14, 12, 10, 8, 6, 4, 2 or less) mg/m² on days 1, 8, and 15 of a 28-day cycle for at least one (such as at least any of 2, 3, 4, 5, 6, 7, 8, 9, 10 or more) cycle. In some embodiments, the romidepsin is administered as an IV dose of about 14 mg/m² on days 1, 8, and 15 of a 28-day cycle for at least one (such as at least any of 2, 3, 4, 5, 6, 7, 8, 9, 10 or more) cycle. The dose of romidepsin may be discontinued or interrupted, with or without dose reduction, to manage adverse drug reactions. In some embodiments, the romidepsin is administered according to the prescribing information of an approved brand of romidepsin.

In some embodiments, according to any of the methods described herein where the second therapeutic agent is nilotinib, the nilotinib is administered as a bi-daily oral dose of about 200 to about 400 mg (including for example about any of 200, 220, 240, 260, 280, 300, 320, 340, 360, 380, or 400 mg, including any range between these values) on days 1-28 of a 28-day cycle for at least one (such as at least any of 2, 3, 4, 5, 6, 7, 8, 9, 10 or more) cycle. In some embodiments, the nilotinib is administered as a bi-daily oral dose of no more than about 400 (such as no more than about any of 400, 350, 300, 250, 200, 150 or less) mg on days 1-28 of a 28-day cycle for at least one (such as at least any of 2, 3, 4, 5, 6, 7, 8, 9, 10 or more) cycle. In some embodiments, the nilotinib is administered as a bi-daily oral dose of about 300 mg on days 1-28 of a 28-day cycle for at least one (such as at least any of 2, 3, 4, 5, 6, 7, 8, 9, 10 or more) cycle. In some embodiments, the nilotinib is administered as a bi-daily oral dose of about 400 mg on days 1-28 of a 28-day cycle for at least one (such as at least any of 2, 3, 4, 5, 6, 7, 8, 9, 10 or more) cycle. In some embodiments, the two daily doses of nilotinib are administered approximately 12 hours apart. The dose of nilotinib may be discontinued or interrupted, with or without dose reduction, to manage adverse drug reactions. In some embodiments, the nilotinib is administered according to the prescribing information of an approved brand of nilotinib.

In some embodiments, according to any of the methods described herein where the second therapeutic agent is sorafenib, the sorafenib is administered as a bi-daily oral dose of about 250 to about 400 mg (including for example about any of 250, 275, 300, 325, 350, 375, or 400 mg, including any range between these values) on days 1-28 of a 28-day cycle for at least one (such as at least any of 2, 3, 4, 5, 6, 7, 8, 9, 10 or more) cycle. In some embodiments, the sorafenib is administered as a bi-daily oral dose of no more than about 400 (such as no more than about any of 400, 375, 350, 325, 300, 275, 250 or less) mg on days 1-28 of a 28-day cycle for at least one (such as at least any of 2, 3, 4, 5, 6, 7, 8, 9, 10 or more) cycle. In some embodiments, the sorafenib is administered as a bi-daily oral dose of about 400 mg on days 1-28 of a 28-day cycle for at least one (such as at least any of 2, 3, 4, 5, 6, 7, 8, 9, 10 or more) cycle. The dose of sorafenib may be discontinued or interrupted, with or without dose reduction, to manage adverse drug reactions. In some embodiments, the sorafenib is administered according to the prescribing information of an approved brand of sorafenib.

Whether administered in therapeutic or sub-therapeutic amounts, the combination of the mTOR inhibitor nanoparticle composition (such as sirolimus/albumin nanoparticle composition) and the second therapeutic agent should be effective in treating a hematological malignancy. For example, a sub-therapeutic amount of an mTOR inhibitor nanoparticle composition (such as sirolimus/albumin nanoparticle composition) can be an effective amount if, when combined with a second therapeutic agent, the combination is effective in the treatment of the hematological malignancy, and vice versa.

The dose of the mTOR inhibitor nanoparticle composition (such as sirolimus/albumin nanoparticle composition) and the dose of the second therapeutic agent administered to an individual (such as a human) may vary with the particular composition, the mode of administration, and the type of disease being treated. In some embodiments, the doses are effective to result in an objective response (such as a partial response or a complete response). In some embodiments, the doses are sufficient to result in a complete response in the individual. In some embodiments, the doses are sufficient to result in a partial response in the individual. In some embodiments, the doses administered are sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the mTOR inhibitor nanoparticle composition (such as sirolimus/albumin nanoparticle composition) and the second therapeutic agent. Responses of an individual to the treatment of the methods described herein can be determined, for example, based on RECIST levels.

In some embodiments, the amounts of the mTOR inhibitor nanoparticle composition (such as sirolimus/albumin nanoparticle composition) and the second therapeutic agent are sufficient to prolong progress-free survival of the individual. In some embodiments, the amounts of the mTOR inhibitor nanoparticle composition (such as sirolimus/albumin nanoparticle composition) and the second therapeutic agent are sufficient to prolong overall survival of the individual. In some embodiments, the amounts of the mTOR inhibitor nanoparticle composition (such as sirolimus/albumin nanoparticle composition) and the second therapeutic agent are sufficient to produce clinical benefit of more than about any of 50%, 60%, 70%, or 77% among a population of individuals treated with the mTOR inhibitor nanoparticle composition and the second therapeutic agent.

In some embodiments, the amounts of the mTOR inhibitor nanoparticle composition (such as sirolimus/albumin nanoparticle composition) and the second therapeutic agent are sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same individual prior to treatment or compared to the corresponding activity in other individuals not receiving the treatment. Standard methods can be used to measure the magnitude of this effect, such as in vitro assays with purified enzyme, cell-based assays, animal models, or human testing.

In some embodiments, the amounts of the mTOR inhibitor nanoparticle composition (such as sirolimus/albumin nanoparticle composition) and the second therapeutic agent are below the levels that induce a toxicological effect (i.e., an effect above a clinically acceptable level of toxicity) or are at a level where a potential side effect can be controlled or tolerated when the mTOR inhibitor nanoparticle composition and the second therapeutic agent are administered to the individual.

In some embodiments, the amount of the mTOR inhibitor nanoparticle composition (such as sirolimus/albumin nanoparticle composition) is close to a maximum tolerated dose (MTD) of the composition following the same dosing regimen when administered with the second therapeutic agent. In some embodiments, the amount of the mTOR inhibitor nanoparticle composition (such as sirolimus/albumin nanoparticle composition) is more than about any of 80%, 90%, 95%, or 98% of the MTD when administered with the second therapeutic agent.

In some embodiments, the amount of an mTOR inhibitor (such as a limus drug, e.g., sirolimus) in the mTOR inhibitor nanoparticle composition is included in any of the following ranges: about 0.1 mg to about 1000 mg, about 0.1 mg to about 2.5 mg, about 0.5 mg to about 5 mg, about 5 mg to about 10 mg, about 10 mg to about 15 mg, about 15 mg to about 20 mg, about 20 mg to about 25 mg, about 20 mg to about 50 mg, about 25 mg to about 50 mg, about 50 mg to about 75 mg, about 50 mg to about 100 mg, about 75 mg to about 100 mg, about 100 mg to about 125 mg, about 125 mg to about 150 mg, about 150 mg to about 175 mg, about 175 mg to about 200 mg, about 200 mg to about 225 mg, about 225 mg to about 250 mg, about 250 mg to about 300 mg, about 300 mg to about 350 mg, about 350 mg to about 400 mg, about 400 mg to about 450 mg, or about 450 mg to about 500 mg, about 500 mg to about 600 mg, about 600 mg to about 700 mg, about 700 mg to about 800 mg, about 800 mg to about 900 mg, or about 900 mg to about 1000 mg, including any range between these values. In some embodiments, the amount of an mTOR inhibitor (such as a limus drug, e.g., sirolimus) in the effective amount of the composition (e.g., a unit dosage form) is in the range of about 5 mg to about 500 mg, such as about 30 to about 400 mg, 30 mg to about 300 mg, or about 50 mg to about 200 mg. In some embodiments, the amount of an mTOR inhibitor (such as a limus drug, e.g., sirolimus) in the effective amount of the mTOR inhibitor nanoparticle composition (e.g., a unit dosage form) is in the range of about 150 mg to about 500 mg, including for example, about 150 mg, about 225 mg, about 250 mg, about 300 mg, about 325 mg, about 350 mg, about 375 mg, about 400 mg, about 425 mg, about 450 mg, about 475 mg, or about 500 mg. In some embodiments, the concentration of the mTOR inhibitor (such as a limus drug, e.g., sirolimus) in the mTOR inhibitor nanoparticle composition is dilute (about 0.1 mg/ml) or concentrated (about 100 mg/ml), including for example about any of 0.1 mg/ml to about 50 mg/ml, about 0.1 mg/ml to about 20 mg/ml, about 1 mg/ml to about 10 mg/ml, about 2 mg/ml to about 8 mg/ml, about 4 mg/ml to about 6 mg/ml, or about 5 mg/ml. In some embodiments, the concentration of the mTOR inhibitor (such as a limus drug, e.g., sirolimus) in the mTOR inhibitor nanoparticle composition is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

In some embodiments of any of the above aspects, the amount of an mTOR inhibitor (such as a limus drug, e.g., sirolimus) in the mTOR inhibitor nanoparticle composition is at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In some embodiments, the effective amount of an mTOR inhibitor (such as a limus drug, e.g., sirolimus) in the mTOR inhibitor nanoparticle composition is less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg.

In some embodiments of any of the above aspects, the amount of an mTOR inhibitor (such as a limus drug, e.g., sirolimus) in the mTOR inhibitor nanoparticle composition is about any of 25 mg/m², 30 mg/m², 50 mg/m², 60 mg/m², 75 mg/m², 80 mg/m², 90 mg/m², 100 mg/m², 120 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² mTOR inhibitor. In some embodiments, the mTOR inhibitor nanoparticle composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² mTOR inhibitor (such as a limus drug, e.g., sirolimus). In some embodiments, the amount of the mTOR inhibitor (such as a limus drug, e.g., sirolimus) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of mTOR inhibitor (such as a limus drug, e.g., sirolimus) in the mTOR inhibitor nanoparticle composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the effective amount of mTOR inhibitor (such as a limus drug, e.g., sirolimus) in the mTOR inhibitor nanoparticle composition is about 30 to about 300 mg/m², such as about 100 to about 150 mg/m², about 120 mg/m2, about 130 mg/m², or about 140 mg/m².

In some embodiments, the combination of compounds exhibits a synergistic effect (i.e., greater than additive effect) in the treatment of the hematological malignancy. The term “synergistic effect” refers to the action of two agents, such as an mTOR inhibitor nanoparticle composition (such as sirolimus/albumin nanoparticle composition) and a second therapeutic agent, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In different embodiments, depending on the combination and the effective amounts used, the combination of compounds can inhibit cancer growth, achieve cancer stasis, or even achieve substantial or complete cancer regression.

While the amounts of an mTOR inhibitor nanoparticle composition (such as sirolimus/albumin nanoparticle composition) and a second therapeutic agent should result in the effective treatment of a hematological malignancy, the amounts, when combined, are preferably not excessively toxic to the individual (i.e., the amounts are preferably within toxicity limits as established by medical guidelines). In some embodiments, either to prevent excessive toxicity and/or provide a more efficacious treatment of a hematological malignancy, a limitation on the total administered dosage is provided.

Different dosage regimens may be used to treat a hematological malignancy. In some embodiments, a daily dosage, such as any of the exemplary dosages described above, is administered once, twice, three times, or four times a day for three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

In some embodiments, the dosing frequencies for the administration of the mTOR inhibitor nanoparticle composition (such as sirolimus/albumin nanoparticle composition) include, but are not limited to, daily, every two days, every three days, every four days, every five days, every six days, weekly without break, three out of four weeks (such as on days 1, 8, and 15 of a 28-day cycle), once every three weeks, once every two weeks, or two out of three weeks. In some embodiments, the mTOR inhibitor nanoparticle composition (such as sirolimus/albumin nanoparticle composition) is administered about once every 2 weeks, once every 3 weeks, once every 4 weeks, once every 6 weeks, or once every 8 weeks. In some embodiments, the mTOR inhibitor nanoparticle composition (such as sirolimus/albumin nanoparticle composition) is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, or eleven times. In some embodiments, the dosing frequency is once every two days for five times. In some embodiments, the mTOR inhibitor (such as a limus drug, e.g., sirolimus or a derivative thereof) is administered over a period of at least ten days, wherein the interval between each administration is no more than about two days, and wherein the dose of the mTOR inhibitor at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m².

The administration of the mTOR inhibitor nanoparticle composition (such as sirolimus/albumin nanoparticle composition) can be extended over an extended period of time, such as from about a month up to about seven years. In some embodiments, the mTOR inhibitor nanoparticle composition (such as sirolimus/albumin nanoparticle composition) is administered over a period of at least about any of 2, 3, 4, 5, 6, 7, 8, 9, 10, 11, 12, 18, 24, 30, 36, 48, 60, 72, or 84 months.

In some embodiments, the dosage of an mTOR inhibitor (such as a limus drug, e.g., sirolimus or a derivative thereof) in a nanoparticle composition can be in the range of 5-400 mg/m² when given on a 3 week schedule, or 5-250 mg/m² (such as 80-150 mg/m², for example 100-120 mg/m²) when given on a weekly schedule. For example, the amount of an mTOR inhibitor (such as a limus drug, e.g., sirolimus or a derivative thereof) is about 60 to about 300 mg/m² (e.g., about 260 mg/m²) on a three week schedule.

In some embodiments, the exemplary dosing schedules for the administration of the mTOR inhibitor nanoparticle composition (such as sirolimus/albumin nanoparticle composition) include, but are not limited to, 100 mg/m², weekly, without break; 10 mg/m² weekly, 3 out of four weeks (such as on days 1, 8, and 15 of a 28-day cycle); 45 mg/m² weekly, 3 out of four weeks (such as on days 1, 8, and 15 of a 28-day cycle); 75 mg/m² weekly, 3 out of four weeks (such as on days 1, 8, and 15 of a 28-day cycle); 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week. The dosing frequency of the mTOR inhibitor nanoparticle composition (such as sirolimus/albumin nanoparticle composition) may be adjusted over the course of the treatment based on the judgment of the administering physician.

In some embodiments, the individual is treated for at least about any of one, two, three, four, five, six, seven, eight, nine, or ten treatment cycles.

The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes.

In some embodiments, the exemplary dose of the mTOR inhibitor (in some embodiments a limus drug, e.g., sirolimus) in the mTOR inhibitor nanoparticle composition includes, but is not limited to, about any of 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m². For example, the dosage of an mTOR inhibitor (such as a limus drug, e.g., sirolimus or a derivative thereof) in a nanoparticle composition can be in the range of about 100-400 mg/m² when given on a 3 week schedule, or about 10-250 mg/m² when given on a weekly schedule.

In some embodiments, the dosage of an mTOR inhibitor (such as a limus drug, e.g., sirolimus) is about 100 mg to about 400 mg, for example about 100 mg, about 200 mg, about 300 mg, or about 400 mg. In some embodiments, the limus drug is administered at about 100 mg weekly, about 200 mg weekly, about 300 mg weekly, about 100 mg twice weekly, or about 200 mg twice weekly. In some embodiments, the administration is further followed by a monthly maintenance dose (which can be the same or different from the weekly doses).

In some embodiments when the limus nanoparticle composition is administered intravenously, the dosage of an mTOR inhibitor (such as a limus drug, e.g., sirolimus) in a nanoparticle composition can be in the range of about 30 mg to about 400 mg. The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes to about 40 minutes.

In some embodiments, each dosage contains both an mTOR inhibitor nanoparticle composition (such as sirolimus/albumin nanoparticle composition) and a second therapeutic agent to be delivered as a single dosage, while in other embodiments, each dosage contains either the mTOR inhibitor nanoparticle composition or the second therapeutic agent to be delivered as separate dosages.

An mTOR inhibitor nanoparticle composition (such as sirolimus/albumin nanoparticle composition) and a second therapeutic agent, in pure form or in an appropriate pharmaceutical composition, can be administered via any of the accepted modes of administration or agents known in the art. The compositions and/or agents can be administered, for example, orally, nasally, parenterally (such as intravenous, intramuscular, or subcutaneous), topically, transdermally, intravaginally, intravesically, intracistemally, or rectally. The dosage form can be, for example, a solid, semi-solid, lyophilized powder, or liquid dosage form, such as tablets, pills, soft elastic or hard gelatin capsules, powders, solutions, suspensions, suppositories, aerosols, or the like, preferably in unit dosage forms suitable for simple administration of precise dosages.

As discussed above, the mTOR inhibitor nanoparticle composition (such as sirolimus/albumin nanoparticle composition) and the second therapeutic agent can be administered in a single unit dose or separate dosage forms. Accordingly, the phrase “pharmaceutical combination” includes a combination of two drugs in either a single dosage form or a separate dosage forms, i.e., the pharmaceutically acceptable carriers and excipients described throughout the application can be combined with an mTOR inhibitor nanoparticle composition (such as sirolimus/albumin nanoparticle composition) and a second therapeutic agent in a single unit dose, as well as individually combined with an mTOR inhibitor nanoparticle composition and a second therapeutic agent when these compounds are administered separately.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as citric acid, sorbitan monolaurate, triethanolamine oleate, butylated hydroxytoluene, and the like.

Solid dosage forms can be prepared with coatings and shells, such as enteric coatings and others well-known in the art. They can contain pacifying agents and can be of such composition that they release the active compound or compounds in a certain part of the intestinal tract in a delayed manner Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the mTOR inhibitor nanoparticle composition (such as sirolimus/albumin nanoparticle composition) or second therapeutic agents described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, such as ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

In some embodiments, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compound described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

The mTOR inhibitor nanoparticle composition (such as sirolimus/albumin nanoparticle composition) can be administered to an individual (such as a human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In some embodiments, the composition is administered intravenously. In some embodiments, the composition is administered intraportally. In some embodiments, the composition is administered intraarterially. In some embodiments, the composition is administered intraperitoneally.

### Nanoparticle Compositions

The mTOR inhibitor nanoparticle compositions described herein comprise nanoparticles comprising (in various embodiments consisting essentially of or consisting of) an mTOR inhibitor (such as a limus drug, e.g., sirolimus or a derivative thereof) and an albumin (such as human serum albumin). Nanoparticles of poorly water soluble drugs (such as macrolides) have been disclosed in, for example, U.S. Pat. Nos. 5,916,596; 6,506,405; 6,749,868, 6,537,579, 7,820,788, and also in U.S. Pat. Pub. Nos. 2006/0263434, and 2007/0082838; PCT Patent Application WO08/137148, each of which is incorporated herein by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 10 to about 400 nm. In some embodiments, the average or mean diameter of the nanoparticles is about 10 to about 150 nm. In some embodiments, the average or mean diameter of the nanoparticles is about 40 to about 120 nm. In some embodiments, the nanoparticles are no less than about 50 nm. In some embodiments, the nanoparticles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 10 nm to about 400 nm, including for example about 10 nm to about 200 nm, about 20 nm to about 200 nm, about 30 nm to about 180 nm, about 40 nm to about 150 nm, about 40 nm to about 120 nm, and about 60 nm to about 100 nm.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprising the mTOR inhibitor (such as a limus drug, e.g., sirolimus or a derivative thereof) are associated (e.g., coated) with an albumin (such as human albumin or human serum albumin) In some embodiments, the composition comprises an mTOR inhibitor (such as a limus drug, e.g., sirolimus or a derivative thereof) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the mTOR inhibitor in the composition are in nanoparticle form. In some embodiments, the mTOR inhibitor (such as a limus drug, e.g., sirolimus or a derivative thereof) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an mTOR inhibitor (such as a limus drug, e.g., sirolimus or a derivative thereof) that is substantially free of polymeric materials (such as polymeric matrix).

In some embodiments, the composition comprises an albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of an albumin (such as human albumin or human serum albumin) and a mTOR inhibitor (such as a limus drug, e.g., sirolimus or a derivative thereof) in the mTOR inhibitor nanoparticle composition is about 18:1 or less, such as about 15:1 or less, for example about 10:1 or less. In some embodiments, the weight ratio of an albumin (such as human albumin or human serum albumin) and an mTOR inhibitor (such as a limus drug, e.g., sirolimus or a derivative thereof) in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of an albumin and an mTOR inhibitor (such as a limus drug, e.g., sirolimus or a derivative thereof) in the nanoparticle portion of the composition is about any one of 1:2, 1:3, 1:4, 1:5, 1:9, 1:10, 1:15, or less. In some embodiments, the weight ratio of the albumin (such as human albumin or human serum albumin) and the mTOR inhibitor (such as a limus drug, e.g., sirolimus or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the mTOR inhibitor nanoparticle composition (such as sirolimus/albumin nanoparticle composition) comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises an albumin (such as human albumin or human serum albumin) The albumin may either be natural in origin or synthetically prepared. In some embodiments, the albumin is human albumin or human serum albumin. In some embodiments, the albumin is a recombinant albumin.

Human serum albumin (HSA) is a highly soluble globular protein of Mr 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys 34), and a single tryptophan (Trp 214). Intravenous use of HSA solution has been indicated for the prevention and treatment of hypovolumic shock (see, e.g., Tullis, JAMA, 237: 355-360, 460-463, (1977)) and Houser et al., Surgery, Gynecology and Obstetrics, 150: 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Rapamycin and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

The albumin (such as human albumin or human serum albumin) in the composition generally serves as a carrier for the mTOR inhibitor, i.e., the albumin in the composition makes the mTOR inhibitor (such as a limus drug, e.g., sirolimus or a derivative thereof) more readily suspendable in an aqueous medium or helps maintain the suspension as compared to compositions not comprising an albumin. This can avoid the use of toxic solvents (or surfactants) for solubilizing the mTOR inhibitor, and thereby can reduce one or more side effects of administration of the mTOR inhibitor (such as a limus drug, e.g., sirolimus or a derivative thereof) into an individual (such as a human). Thus, in some embodiments, the composition described herein is substantially free (such as free) of surfactants, such as Cremophor (or polyoxyethylated castor oil, including Cremophor EL® (BASF)). In some embodiments, the mTOR inhibitor nanoparticle composition (such as sirolimus/albumin nanoparticle composition) is substantially free (such as free) of surfactants. A composition is “substantially free of Cremophor” or “substantially free of surfactant” if the amount of Cremophor or surfactant in the composition is not sufficient to cause one or more side effect(s) in an individual when the mTOR inhibitor nanoparticle composition (such as sirolimus/albumin nanoparticle composition) is administered to the individual. In some embodiments, the mTOR inhibitor nanoparticle composition (such as sirolimus/albumin nanoparticle composition) contains less than about any one of 20%, 15%, 10%, 7.5%, 5%, 2.5%, or 1% organic solvent or surfactant. In some embodiments, the albumin is human albumin or human serum albumin. In some embodiments, the albumin is recombinant albumin.

The amount of an albumin in the composition described herein will vary depending on other components in the composition. In some embodiments, the composition comprises an albumin in an amount that is sufficient to stabilize the mTOR inhibitor (such as a limus drug, e.g., sirolimus or a derivative thereof) in an aqueous suspension, for example, in the form of a stable colloidal suspension (such as a stable suspension of nanoparticles). In some embodiments, the albumin is in an amount that reduces the sedimentation rate of the mTOR inhibitor (such as a limus drug, e.g., sirolimus or a derivative thereof) in an aqueous medium. For particle-containing compositions, the amount of the albumin also depends on the size and density of nanoparticles of the mTOR inhibitor.

An mTOR inhibitor (such as a limus drug, e.g., sirolimus or a derivative thereof) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as a human) Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin is present in an amount that is sufficient to stabilize the mTOR inhibitor (such as a limus drug, e.g., sirolimus or a derivative thereof) in an aqueous suspension at a certain concentration. For example, the concentration of the mTOR inhibitor (such as a limus drug, e.g., sirolimus or a derivative thereof) in the composition is about 0.1 to about 100 mg/ml, including for example about any of 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the mTOR inhibitor (such as a limus drug, e.g., sirolimus or a derivative thereof) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), or about 50% (w/v)) of albumin. In some embodiments, the composition, in liquid form, comprises about 0.5% to about 5% (w/v) of albumin.

In some embodiments, the weight ratio of the albumin to the mTOR inhibitor (such as a limus drug, e.g., sirolimus or a derivative thereof) in the mTOR inhibitor nanoparticle composition is such that a sufficient amount of mTOR inhibitor binds to, or is transported by, the cell. While the weight ratio of the albumin to the mTOR inhibitor (such as a limus drug, e.g., sirolimus or a derivative thereof) will have to be optimized for different albumin and mTOR inhibitor combinations, generally the weight ratio of an albumin to an mTOR inhibitor (such as a limus drug, e.g., sirolimus or a derivative thereof) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to mTOR inhibitor (such as a limus drug, e.g., sirolimus or a derivative thereof) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the mTOR inhibitor (such as a limus drug, e.g., sirolimus or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the composition to be administered to an individual (such as a human) without significant side effects. albumin (such as human serum albumin or human albumin) is in an amount that is effective to reduce one or more side effects of administration of the mTOR inhibitor (such as a limus drug, e.g., sirolimus or a derivative thereof) to a human. The term “reducing one or more side effects” of administration of the mTOR inhibitor (such as a limus drug, e.g., sirolimus or a derivative thereof) refers to reduction, alleviation, elimination, or avoidance of one or more undesirable effects caused by the mTOR inhibitor, as well as side effects caused by delivery vehicles (such as solvents that render the limus drugs suitable for injection) used to deliver the mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as a limus drug, e.g., sirolimus or a derivative thereof) can be reduced.

In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus and human albumin (such as human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus and human albumin (such as human serum albumin), wherein the average or mean diameter of the nanoparticles is about 10 to about 150 nm. In some embodiments, the mTOR inhibitor nanoparticle compositions described herein comprise nanoparticles comprising sirolimus and human albumin (such as human serum albumin), wherein the average or mean diameter of the nanoparticles is about 40 to about 120 nm.

In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus and human albumin (such as human serum albumin), wherein the nanoparticles have an average diameter of no greater than about 150 nm (for example about 100 nm), wherein the weight ratio of albumin and mTOR inhibitor in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 10 nm to about 150 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 40 nm to about 120 nm. In some embodiments, the mTOR inhibitor nanoparticle compositions described herein comprise nanoparticles comprising sirolimus associated (e.g., coated) with human albumin (such as human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus associated (e.g., coated) with human albumin (such as human serum albumin), wherein the nanoparticles have an average diameter of about 10 nm to about 150 nm. In some embodiments, the mTOR inhibitor nanoparticle compositions described herein comprise nanoparticles comprising sirolimus associated (e.g., coated) with human albumin (such as human serum albumin), wherein the nanoparticles have an average diameter of about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus associated (e.g., coated) with human albumin (such as human serum albumin), wherein the nanoparticles have an average diameter of no greater than about 150 nm (for example about 100 nm), wherein the weight ratio of albumin and the sirolimus in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus stabilized by human albumin (such as human serum albumin), wherein the nanoparticles have an average diameter of no greater than about 150 nm (for example about 100 nm). In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus stabilized by human albumin (such as human serum albumin), wherein the nanoparticles have an average diameter of no greater than about 150 nm (for example about 100 nm), wherein the weight ratio of albumin and the sirolimus in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle composition comprises nab-sirolimus. In some embodiments, the mTOR inhibitor nanoparticle composition is nab-sirolimus. nab-sirolimus is a formulation of sirolimus stabilized by human albumin USP, which can be dispersed in directly injectable physiological solution. The weight ratio of human albumin and sirolimus is about 8:1 to about 9:1. When dispersed in a suitable aqueous medium such as 0.9% sodium chloride injection or 5% dextrose injection, nab-sirolimus forms a stable colloidal suspension of sirolimus. The mean particle size of the nanoparticles in the colloidal suspension is about 100 nanometers. Since HSA is freely soluble in water, nab-sirolimus can be reconstituted in a wide range of concentrations ranging from dilute (0.1 mg/ml sirolimus or a derivative thereof) to concentrated (20 mg/ml sirolimus or a derivative thereof), including for example about 2 mg/ml to about 8 mg/ml, or about 5 mg/ml.

Methods of making nanoparticle compositions are known in the art. For example, nanoparticles containing an mTOR inhibitor (such as a limus drug, e.g., sirolimus or a derivative thereof) and an albumin (such as human serum albumin or human albumin) can be prepared under conditions of high shear forces (e.g., sonication, high pressure homogenization, or the like). These methods are disclosed in, for example, U.S. Pat. Nos. 5,916,596; 6,506,405; 6,749,868, 6,537,579 and 7,820,788 and also in U.S. Pat. Pub. Nos. 2007/0082838, 2006/0263434 and PCT Application WO08/137148.

Briefly, the mTOR inhibitor (such as a limus drug, e.g., sirolimus or a derivative thereof) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

mTOR Inhibitor

The methods described herein in some embodiments comprise administration of nanoparticle compositions of mTOR inhibitors. “mTOR inhibitor” used herein refers to an inhibitor of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8), PRAS40 and DEPTOR (Kim et al. (2002). Cell 110: 163-75; Fang et al. (2001). Science 294 (5548): 1942-5). mTORC1 integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Ragulator (LAMTOR1-3) Growth factors and hormones (e.g., insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GβL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase C α (PKCα). It had been observed that knocking down mTORC2 components affects actin polymerization and perturbs cell morphology (Jacinto et al. (2004). Nat. Cell Biol. 6, 1122-1128; Sarbassov et al. (2004). Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the mTOR inhibitor (such as a limus drug, e.g., sirolimus or a derivative thereof) is an inhibitor of mTORC1. In some embodiments, the mTOR inhibitor (such as a limus drug, e.g., sirolimus or a derivative thereof) is an inhibitor of mTORC2. In some embodiments, the mTOR inhibitor (such as a limus drug, e.g., sirolimus or a derivative thereof) is an inhibitor of both mTORC1 and mTORC2.

In some embodiments, the mTOR inhibitor is a limus drug, which includes sirolimus and its analogues. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

In some embodiments, the mTOR inhibitor is sirolimus. Sirolimus is macrolide antibiotic that complexes with FKBP-12 and inhibits the mTOR pathway by binding mTORC1.

In some embodiments, the mTOR inhibitor is selected from the group consisting of sirolimus (rapamycin), BEZ235 (NVP-BEZ235), everolimus (also known as RAD001, Zortress, Certican, and Afinitor), AZD8055,temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

BEZ235 (NVP-BEZ235) is an imidazoquilonine derivative that is an mTORC1 catalytic inhibitor (Roper J, et al. PLoS One, 2011, 6(9), e25132). Everolimus is the 40-O-(2-hydroxyethyl) derivative of sirolimus and binds the cyclophilin FKBP-12, and this complex also mTORC1. AZD8055 is a small molecule that inhibits the phosphorylation of mTORC1 (p70S6K and 4E-BP1). Temsirolimus is a small molecule that forms a complex with the FK506-binding protein and prohibits the activation of mTOR when it resides in the mTORC1complex. PI-103 is a small molecule that inhibits the activation of the rapamycin-sensitive (mTORC1) complex (Knight et al. (2006) Cell. 125: 733-47). KU-0063794 is a small molecule that inhibits the phosphorylation of mTORC1 at Ser2448 in a dose-dependent and time-dependent manner. INK 128, AZD2014, NVP-BGT226, CH5132799, WYE-687, and are each small molecule inhibitors of mTORC1. PF-04691502 inhibits mTORC1 activity. GDC-0980 is an orally bioavailable small molecule that inhibits Class I PI3 Kinase and TORC1. Torin 1 is a potent small molecule inhibitor of mTOR. WAY-600 is a potent, ATP-competitive and selective inhibitor of mTOR. WYE-125132 is an ATP-competitive small molecule inhibitor of mTORC1. GSK2126458 is an inhibitor of mTORC1. PKI-587 is a highly potent dual inhibitor of PI3Kα, PI3Kγ and mTOR. PP-121 is a multi-target inhibitor of PDGFR, Hck, mTOR, VEGFR2, Src and Abl. OSI-027 is a selective and potent dual inhibitor of mTORC1 and mTORC2 with IC50 of 22 nM and 65 nM, respectively. Palomid 529 is a small molecule inhibitor of mTORC1 that lacks affinity for ABCB1/ABCG2 and has good brain penetration (Lin et al. (2013) Int J Cancer DOI: 10.1002/ijc. 28126 (e-published ahead of print). PP242 is a selective mTOR inhibitor. XL765 is a dual inhibitor of mTOR/PI3k for mTOR, p110α, p110β, p110γ and p110δ. GSK1059615 is a novel and dual inhibitor of PI3Kα, PI3Kβ, PI3Kδ, PI3Kγ and mTOR. WYE-354 inhibits mTORC1 in HEK293 cells (0.2 μM-5 μM) and in HUVEC cells (10 nM-1 μM). WYE-354 is a potent, specific and ATP-competitive inhibitor of mTOR. Deforolimus (Ridaforolimus, AP23573, MK-8669) is a selective mTOR inhibitor.

Other Components in the mTOR Inhibitor Nanoparticle Compositions

The nanoparticles described herein can be present in a composition that include other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the mTOR inhibitor nanoparticle composition (such as sirolimus/albumin nanoparticle composition) (see, e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Immunomodulators

The methods described herein in some embodiments comprise administration of nanoparticle compositions of mTOR inhibitors in combination with an immunomodulator. “Immunomodulator” used herein refers to a therapeutic agent that when present, alters, suppresses or stimulates the body's immune system Immunomodulators can include compositions or formulations that activate the immune system (e.g., adjuvants or activators), or downregulate the immune system. Adjuvants can include aluminum-based compositions, as well as compositions that include bacterial or mycobacterial cell wall components. Activators can include molecules that activate antigen presenting cells to stimulate the cellular immune response. For example, activators can be immunostimulant peptides. Activators can include, but are not limited to, agonists of toll-like receptors TLR-2, 3, 4, 6, 7, 8, or 9, granulocyte macrophage colony stimulating factor (GM-CSF); TNF; CD40L; CD28; FLT-3 ligand; or cytokines such as IL-1, IL-2, IL-4, IL-7, IL-12, IL-15, or IL-21. Activators can include agonists of activating receptors (including co-stimulatory receptors) on T cells, such as an agonist (e.g., agonistic antibody) of CD28, OX40, ICOS, GITR, 4-1BB, CD27, CD40, or HVEM. Activators can also include compounds that inhibit the activity of an immune suppressor, such as an inhibitor of the immune suppressors IL-10, IL-35, FasL, TGF-β, indoleamine-2,3 dioxygenase (IDO), or cyclophosphamide, or inhibit the activity of an immune checkpoint such as an antagonist (e.g., antagonistic antibody) of CTLA4, PD-1, PD-L1, PD-L2, LAG3, B7-1, B7-H3, B7-H4, BTLA, VISTA, KIR, A2aR, or TIM3. Activators can also include costimulatory molecules such as CD40, CD80, or CD86. Immunomodulators can also include agents that downregulate the immune system such as antibodies against IL-12p70, antagonists of toll-like receptors TLR-2, 3, 4, 5, 6, 8, or 9, or general suppressors of immune function such as cyclophosphamide, cyclosporin A or FK506. Other antibodies of interest include those directed to tumor cell targets, including for example anti-CD38 antibody (such as daratumumab). These agents (e.g., adjuvants, activators, or downregulators) can be combined to shape an optimal immune response.

The indoleamine-2,3 dioxygenase (IDO) enzyme catalyzes the breakdown of the essential amino acid tryptophan, and has emerged as a key target in cancer immunotherapy because of its role in enabling cancers to evade the immune system. IDO activity leads to a tryptophan deficit, which starves cytotoxic T-cells within the tumor microenvironment. Additionally, the resulting tryptophan metabolites activate regulatory T-cells, which further suppresses the immune response to the tumor. IDO is overexpressed by antigen presenting cells in many cancers, and high IDO expression appears to correlate with poor outcome in a number of cancers, including ovarian cancer, AML, endometrial carcinoma, colon cancer, and melanoma. Blocking IDO enhances immune response against tumors. IDO inhibitors include, but are not limited to, small molecule or antibody-based inhibitors, such as 1-methyl-[D]-tryptophan (D-1MT, NSC-721782), epacadostat (INCB24360), norharmane (β-Carboline), rosmarinic acid, and COX-2 inhibitors.

As used herein, the term “immune checkpoint inhibitors,” “checkpoint inhibitors,” and the like refers to compounds that inhibit the activity of control mechanisms of the immune system Immune system checkpoints, or immune checkpoints, are inhibitory pathways in the immune system that generally act to maintain self-tolerance or modulate the duration and amplitude of physiological immune responses to minimize collateral tissue damage. Checkpoint inhibitors can inhibit an immune system checkpoint by inhibiting the activity of a protein in the pathway. Immune system checkpoint proteins include, but are not limited to, cytotoxic T-lymphocyte antigen 4 (CTLA4), programmed cell death 1 protein (PD-1), programmed cell death 1 ligand 1 (PD-L1), programmed cell death 1 ligand 2 (PD-L2), lymphocyte activation gene 3 (LAG3), B7-1, B7-H3, B7-H4, T cell membrane protein 3 (TIM3), B- and T-lymphocyte attenuator (BTLA), V-domain immunoglobulin (Ig)-containing suppressor of T-cell activation (VISTA), Killer-cell immunoglobulin-like receptor (KIR), and A2A adenosine receptor (A2aR). As such, checkpoint inhibitors include antagonists of CTLA4, PD-1, PD-L1, PD-L2, LAG3, B7-1, B7-H3, B7-H4, BTLA, VISTA, KIR, A2aR, or TIM3. For example, antibodies that bind to CTLA4, PD-1, PD-L1, PD-L2, LAG3, B7-1, B7-H3, B7-H4, BTLA, VISTA, KIR, A2aR, or TIM3 and antagonize their function are checkpoint inhibitors. Moreover, any molecule (e.g., peptide, nucleic acid, small molecule, etc.) that inhibits the inhibitory function of an immune system checkpoint is an immune checkpoint inhibitor.

Sirolimus, derivate thereof, and other mTOR inhibitors are generally regarded as immunosuppressive agents and therefore there has been no interest in combining immuno-oncology antibody drugs (for example, anti-PD-1 or anti-PD-L1) with mTOR inhibitors, since the main goal of those therapies is to activate the immune system against the target cells or disease. We propose, however, that the use of mTOR inhibitors, specifically ABI-009 (albumin-bound nanoparticles of sirolimus) may activate the immune system, including for example T cells, such as CD8⁺ T cells or memory T cells, to further improve the activity of these immune-oncology agents against the disease.

CTLA-4 is an immune checkpoint molecule, which is up-regulated on activated T-cells. An anti-CTLA4 mAb can block the interaction of CTLA-4 with CD80/86 and switch off the mechanism of immune suppression and enable continuous stimulation of T-cells by DCs. Two IgG mAb directed against CTLA-4, ipilimumab and tremelimumab, have been tested in clinical trials for a number of indications. Ipilimumab is approved by the FDA for the treatment of melanoma.

PD-1 is a part of the B7/CD28 family of co-stimulatory molecules that regulate T-cell activation and tolerance, and thus antagonistic anti-PD-1 antibodies can be useful for overcoming tolerance. Engagement of the PD-1/PD-L1 pathway results in inhibition of T-cell effector function, cytokine secretion and proliferation. (Turnis et al., OncoImmunology 1(7):1172-1174, 2012). High levels of PD-1 are associated with exhausted or chronically stimulated T cells. Moreover, increased PD-1 expression correlates with reduced survival in cancer patients. Nivolumab is a human mAb to PD-1 that is FDA approved for the treatment of unresectable or metastatic melanoma, as well as squamous non-small cell lung cancer.

In some embodiments, according to any of the methods described above, the immunomodulator enhances an immune response in the individual and may include, but is not limited to, a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, or pomalidomide. In some embodiments, the immunomodulator is pomalidomide or an enantiomer or a mixture of enantiomers thereof, or a pharmaceutically acceptable salt, solvate, hydrate, co-crystal, clathrate, or polymorph thereof. In some embodiments, the immunomodulator is lenalidomide or an enantiomer or a mixture of enantiomers thereof, or a pharmaceutically acceptable salt, solvate, hydrate, co-crystal, clathrate, or polymorph thereof.

In some embodiments, according to any of the methods described above, the immunomodulator enhances an immune response in the individual and may include, but is not limited to, an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MEDI4736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-IL-35, anti-FasL, and anti-TGF-β (such as Fresolumimab). In some embodiments, the antibody is an antagonistic antibody. In some embodiments, the antibody is a monoclonal antibody. In some embodiments, the antibody is human or humanized.

In some embodiments, according to any of the methods described above, the immunomodulator enhances an immune response in the individual and may include, but is not limited to, an antibody selected from the group consisting of anti-CD28, anti-OX40 (such as MEDI6469), anti-ICOS (such as JTX-2011, Jounce Therapeutics), anti-GITR (such as TRX518), anti-4-1BB (such as BMS-663513 and PF-05082566), anti-CD27 (such as Varlilumab and hCD27.15), anti-CD40 (such as CP870,893), and anti-HVEM. In some embodiments, the antibody is an agonistic antibody. In some embodiments, the antibody is a monoclonal antibody. In some embodiments, the antibody is human or humanized.

Thus, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator. In some embodiments, the immunomodulator is an immunostimulator. In some embodiments, the immunostimulator directly stimulates the immune system. In some embodiments, the immunomodulator is an IMiDs® (Celgene). IMiDs® compounds are proprietary small molecule, orally available compounds that modulate the immune system and other biological targets through multiple mechanisms of action. In some embodiments, the immunomodulator is small molecule or antibody-based IDO inhibitor. In some embodiments, the immunomodulator is selected from the group consisting of a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, and pomalidomide. In some embodiments, the immunomodulator is lenalidomide or an enantiomer or a mixture of enantiomers thereof, or a pharmaceutically acceptable salt, solvate, hydrate, co-crystal, clathrate, or polymorph thereof. In some embodiments, the immunomodulator is pomalidomide or an enantiomer or a mixture of enantiomers thereof, or a pharmaceutically acceptable salt, solvate, hydrate, co-crystal, clathrate, or polymorph thereof. In some embodiments, the immunomodulator is an agonistic antibody that targets an activating receptor (including co-stimulatory receptors) on a T cell. In some embodiments, the immunomodulator is an agonistic antibody selected from the group consisting of anti-CD28, anti-OX40 (such as MEDI6469), anti-ICOS (such as JTX-2011, Jounce Therapeutics), anti-GITR (such as TRX518), anti-4-1BB (such as BMS-663513 and PF-05082566), anti-CD27 (such as Varlilumab and hCD27.15), anti-CD40 (such as CP870,893), and anti-HVEM. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MEDI4736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-MR (such as Lirilumab and IPH2101), anti-A2aR, anti-CD52 (such as alemtuzumab), anti-IL-10, anti-IL-35, anti-FasL, and anti-TGF-β (such as Fresolumimab).

In some embodiments, the immunomodulator is an immunostimulator. In some embodiments, the immunomodulator is an immunostimulator that directly stimulates the immune system of the individual. In some embodiments, the immunomodulator is an immune checkpoint inhibitor. In some embodiments, the immune checkpoint inhibitor is an antagonistic antibody that targets an immune checkpoint protein. In some embodiments, the immunomodulator is selected from the group consisting of a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, and pomalidomide. In some embodiments, the immunomodulator is pomalidomide or an enantiomer or a mixture of enantiomers thereof, or a pharmaceutically acceptable salt, solvate, hydrate, co-crystal, clathrate, or polymorph thereof. In some embodiments, the immunomodulator is lenalidomide or an enantiomer or a mixture of enantiomers thereof, or a pharmaceutically acceptable salt, solvate, hydrate, co-crystal, clathrate, or polymorph thereof. In some embodiments, the immunomodulator is an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MEDI4736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-IL-35, anti-FasL, and anti-TGF-β (such as Fresolumimab).

In some embodiments, the immunomodulator is a compound of Formula I:

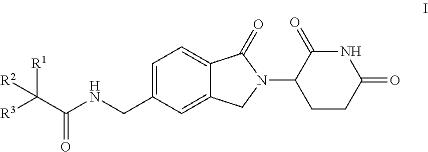

or an enantiomer or a mixture of enantiomers thereof, or a pharmaceutically acceptable salt, solvate, hydrate, co-crystal, clathrate, or polymorph thereof, wherein:

R¹ is H, optionally substituted alkyl, optionally substituted cycloalkyl, optionally substituted aryl, optionally substituted heteroaryl or optionally substituted heterocyclyl;

R² and R³ are each halo;

where the substituents on R′, when present are one to three groups Q, where Q is alkyl, halo, haloalkyl, hydroxyl, alkoxy, cycloalkyl, cycloalkylalkyl, —R⁴OR⁵, —R⁴SR⁵, —R⁴N(R⁶)(R⁷), —R⁴OR⁴N(R⁶)(R⁷) or —R⁴OR⁴C(J)N(R⁶)(R⁷);

each R⁴ is independently alkylene, alkenylene or a direct bond;

each R⁵ is independently hydrogen, alkyl, haloalkyl or hydroxyalkyl; and R⁶ and R⁷ are each independently hydrogen or alkyl.

In some embodiments, the immunomodulator is a compound of Formula I or an enantiomer or a mixture of enantiomers thereof, or a pharmaceutically acceptable salt, solvate, hydrate, co-crystal, clathrate, or polymorph thereof, wherein:

R¹ is optionally substituted alkyl, optionally substituted cycloalkyl, optionally substituted aryl, optionally substituted heteroaryl or optionally substituted heterocyclyl;

R² and R³ are each halo;

where the substituents on R′, when present are one to three groups Q, where Q is alkyl, halo, haloalkyl, hydroxyl, alkoxy, cycloalkyl; cycloalkylalkyl, —R⁴OR⁵, —R⁴SR⁵, —R⁴N(R⁶)(R⁷), —R⁴OR⁴N(R⁶)(R⁷) or —R⁴OR⁴C(J)N(R⁶)(R⁷);

each R¹ is independently alkylene, alkenylene or a direct bond;

each R⁵ is independently hydrogen, alkyl, haloalkyl or hydroxyalkyl; and

R⁶ and R⁷ are each independently hydrogen or alkyl.

In some embodiments, the immunomodulator is a compound selected from the group consisting of:

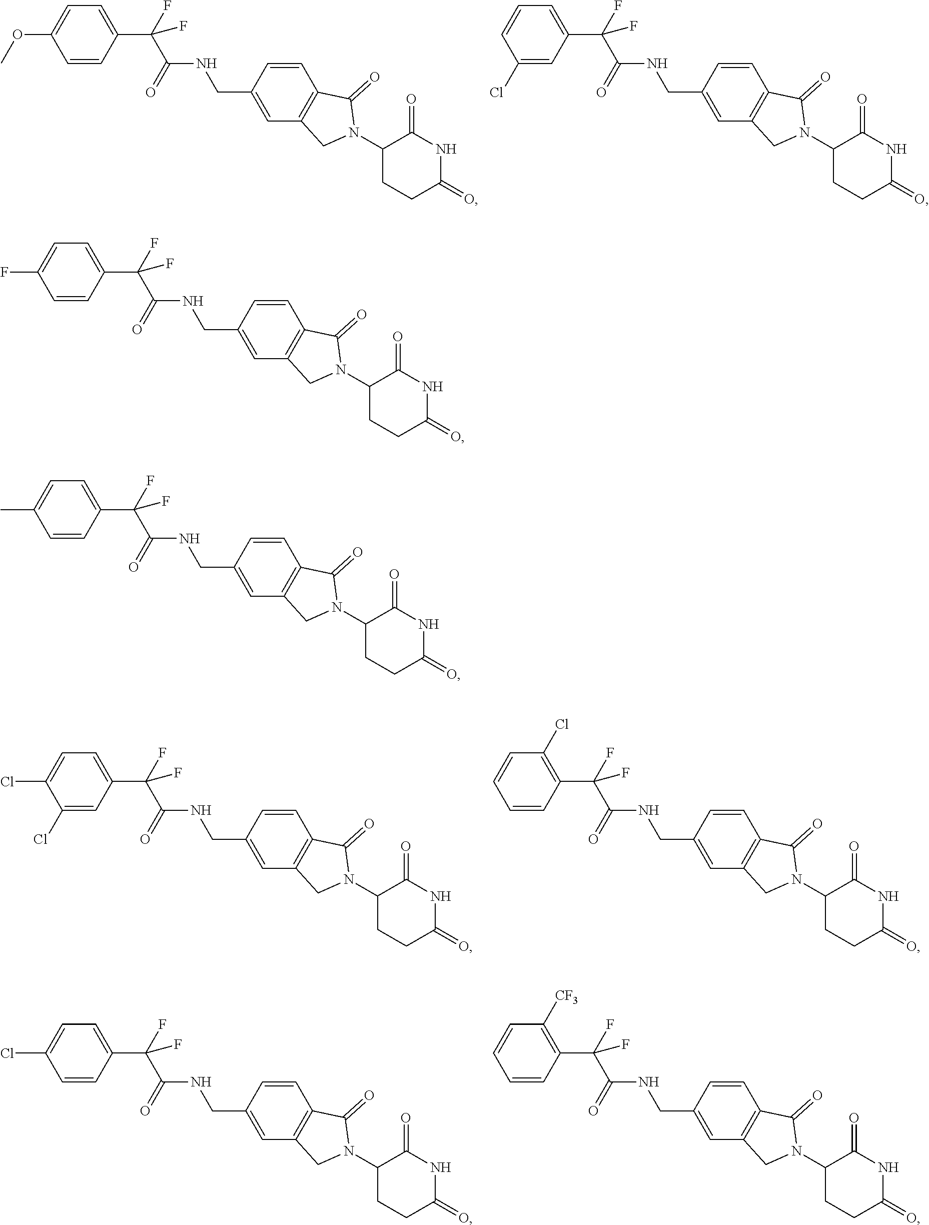

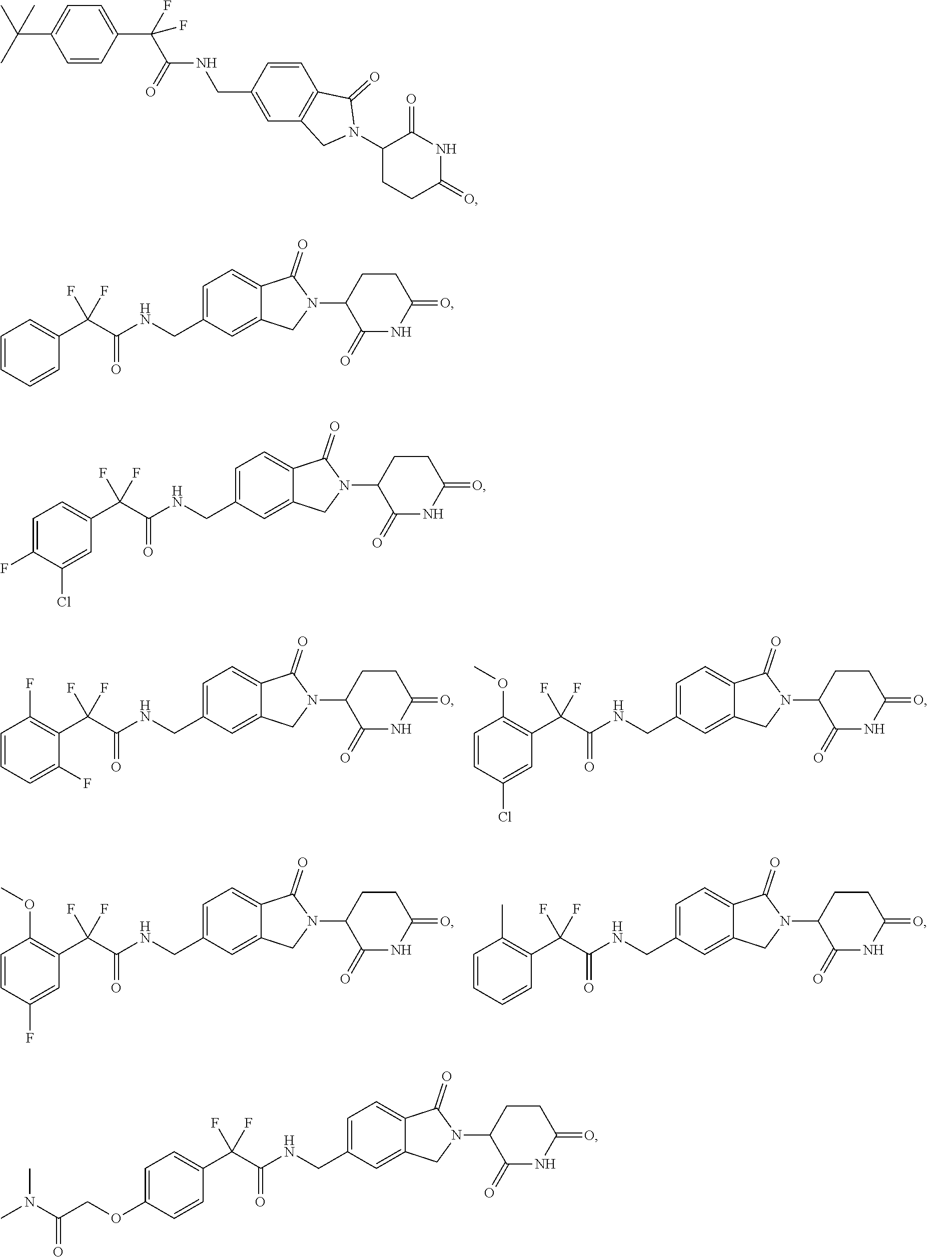

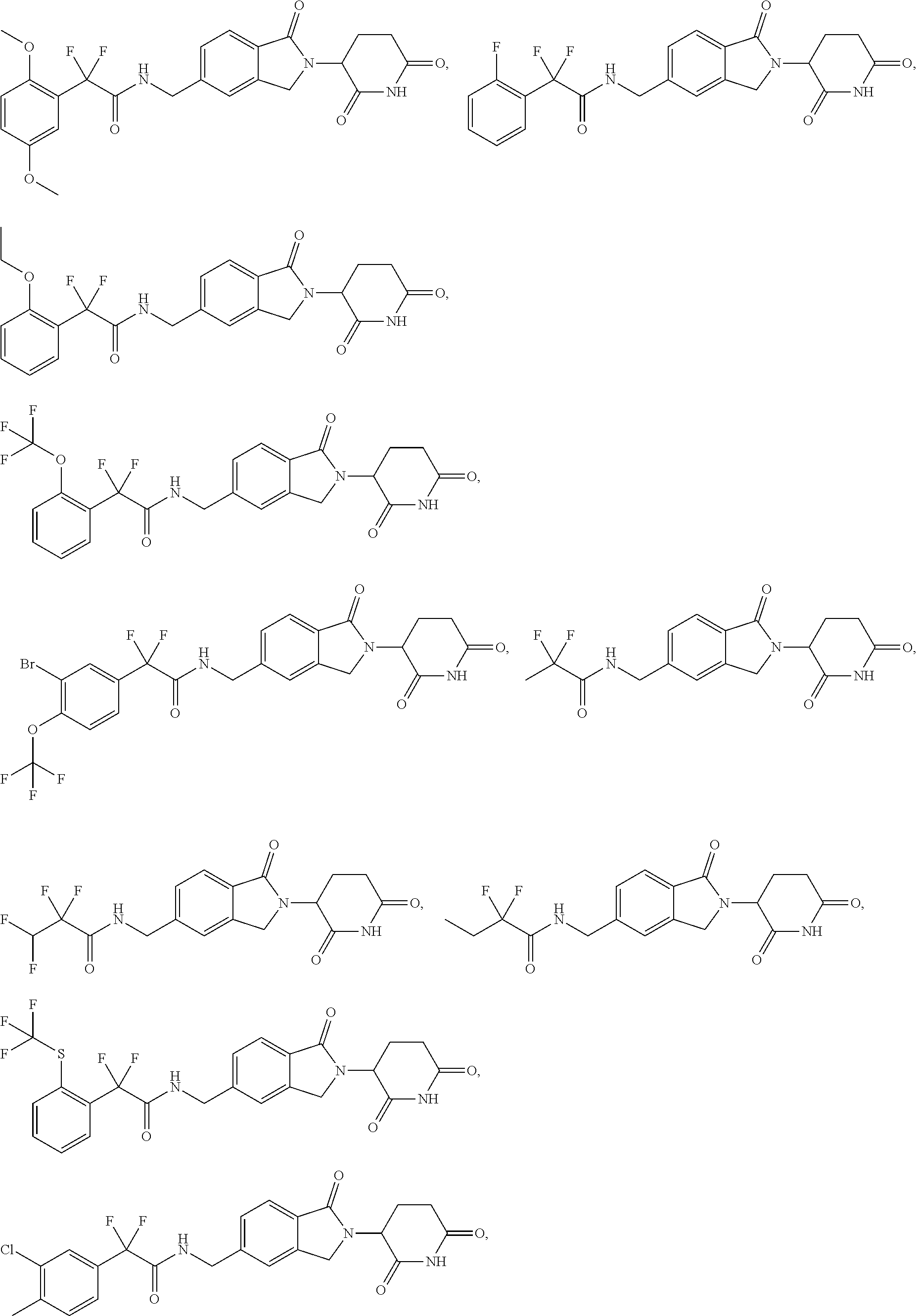

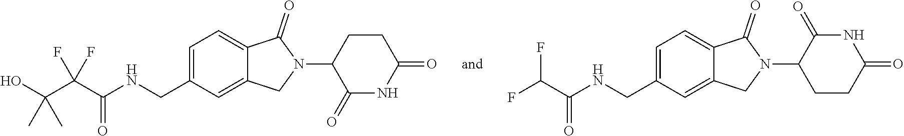 [SMILES: CC(C)(O)C(F)(F)C(=O)NCc1ccc2c(c1)CN(C1CCC(=O)NC1=O)C2=O ; O=C1CCC(N2Cc3cc(CNC(=O)C(F)F)ccc3C2=O)C(=O)N1]

In some embodiments, the immunomodulator is an arylmethoxy isoindoline compound. Specific arylmethoxy isoindoline compounds provided herein include, but are not limited to, compounds such as those described in U.S. Pat. No. 8,518,972, which is incorporated herein by reference in its entirety. In some embodiments, representative arylmethoxy isoindoline compounds are of Formula II:

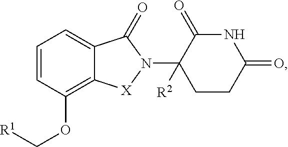

or a pharmaceutically acceptable salt or stereoisomer thereof, wherein:

### X is C=0 or CH₂;

R¹ is —Y—R³;
R² is H or (C₁-C₆)alkyl;
R³ is: —(CH₂)ₙ-aryl, —O—(CH₂)ₙ-aryl or —(CH₂)ₙ—O-aryl, wherein the aryl is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; —(CH₂),-heterocycle, —O—(CH₂)ₙ-heterocycle or —(CH₂)₁, —O-heterocycle, wherein the heterocycle is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; or —(CH₂)ₙ-heteroaryl, —O—(CH₂)ₙ-heteroaryl or —(CH₂)ₙ—O-heteroaryl, wherein the heteroaryl is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; and n is 0, 1, 2 or 3.

In some embodiments, the immunomodulator is a compound of Formula II having the formula:

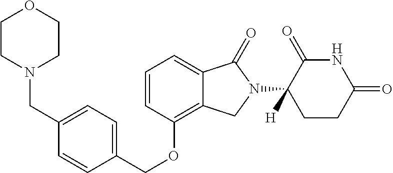 [SMILES: O=C1CC[C@H](N2Cc3c(OCc4ccc(CN5CCOCC5)cc4)cccc3C2=O)C(=O)N1]

In some embodiments, the immunomodulator is a substituted quinazolinone compound. Specific substituted quinazolinone compounds provided herein include, but are not limited to, compounds such as those described in U.S. Pat. No. 7,635,700, U.S. Patent Publication No. 2012/0230983, published Sep. 13, 2012, and U.S. Patent Publication No. 2014/0328832, published Nov. 6, 2014, each of which is incorporated herein by reference in its entirety. In some embodiments, representative substituted quinazolinone compounds are of Formula III:

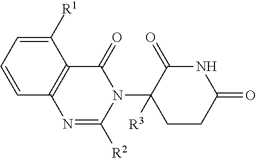

and pharmaceutically acceptable salts, solvates, and stereoisomers thereof, wherein: R¹ is: hydrogen; halo; —(CH₂)ₙOH; (C₁-C₆)alkyl, optionally substituted with one or more halo; (C₁-C₆)alkoxy, optionally substituted with one or more halo; or —(CH₂)ₙNHR^{a}, wherein R^{a} is: hydrogen; (C₁-C₆)alkyl, optionally substituted with one or more halo; —(CH₂)ₙ-(6 to 10 membered aryl); —C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo; —C(O)—(C₁-C₈)alkyl, wherein the alkyl is optionally substituted with one or more halo; —C(O)—(CH₂)ₙ—(C₃-C₁₀-cycloalkyl); —C(O)—(CH₂)ₙ—NR^{b}R^{e}, wherein R^{b} and R^{e} are each independently: hydrogen; (C₁-C₆)alkyl, optionally substituted with one or more halo; (C₁-C₆)alkoxy, optionally substituted with one or more halo; or 6 to 10 membered aryl, optionally substituted with one or more of: halo; (C₁-C₆)alkyl, itself optionally substituted with one or more halo; or (C₁-C₆)alkoxy, itself optionally substituted with one or more halo; —C(O)—(CH₂)ₙ—O—(C₁-C₆)alkyl; or —C(O)—(CH₂)ₙ—O—(CH₂)ₙ-(6 to 10 membered aryl);

R² is: hydrogen; —(CH₂)ₙOH; phenyl; —O—(C₁-C₆)alkyl; or (C₁-C₆)alkyl, optionally substituted with one or more halo;
R³ is: hydrogen; or (C₁-C₆)alkyl, optionally substituted with one or more halo; and n is 0, 1, or 2.

In some embodiments, representative substituted quinazolinone compounds are of Formula IV:

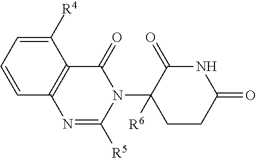

and pharmaceutically acceptable salts, solvates, and stereoisomers thereof, wherein:
R⁴ is: hydrogen; halo; —(CH₂)ₙOH; (C₁-C₆)alkyl, optionally substituted with one or more halo; or (C₁-C₆)alkoxy, optionally substituted with one or more halo;
R⁵ is: hydrogen; —(CH₂)ₙOH; phenyl; —O—(C₁-C₆)alkyl; or (C₁-C₆)alkyl, optionally substituted with one or more halo;
R⁶ is: hydrogen; or (C₁-C₆)alkyl, optionally substituted with one or more halo; and n is 0, 1, or 2.

In one embodiment, R⁴ is hydrogen. In another embodiment, R⁴ is halo. In another embodiment, R⁴ is (C₁-C₆)alkyl, optionally substituted with one or more halo. In another embodiment, R⁴ is —(CH₂)ₙOH or hydroxyl. In another embodiment, R⁴ is (C₁-C₆)alkoxy, optionally substituted with one or more halo.

In one embodiment, R⁵ is hydrogen. In another embodiment, R⁵ is —(CH₂)ₙOH or hydroxyl. In another embodiment, R⁵ is phenyl. In another embodiment, R⁵ is —O—(C₁-C₆)alkyl, optionally substituted with one or more halo. In another embodiment, R⁵ is (C₁-C₆)alkyl, optionally substituted with one or more halo.

In one embodiment, R⁶ is hydrogen. In another embodiment, R⁶ is (C₁-C₆)alkyl, optionally substituted with one or more halo.

In one embodiment, n is 0. In another embodiment, n is 1. In another embodiment, n is 2.

Compounds provided herein encompass any of the combinations of R⁴, R⁵, R⁶ and n described above.

In one specific embodiment, R⁴ is methyl. In another embodiment, R⁴ is methoxy. In another embodiment, R⁴ is —CF3. In another embodiment, R⁴ is F or Cl.

In another specific embodiment, R⁵ is methyl. In another embodiment, R⁵ is —CF3.

Thus, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator selected from the group consisting of compounds of Formula I-IV. In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a compound of Formula I, or an enantiomer or a mixture of enantiomers thereof, or a pharmaceutically acceptable salt, solvate, hydrate, co-crystal, clathrate, or polymorph thereof. In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a compound of Formula II, or an enantiomer or a mixture of enantiomers thereof, or a pharmaceutically acceptable salt, solvate, hydrate, co-crystal, clathrate, or polymorph thereof. In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a compound of Formula III, or an enantiomer or a mixture of enantiomers thereof, or a pharmaceutically acceptable salt, solvate, hydrate, co-crystal, clathrate, or polymorph thereof. In some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a compound of Formula IV, or an enantiomer or a mixture of enantiomers thereof, or a pharmaceutically acceptable salt, solvate, hydrate, co-crystal, clathrate, or polymorph thereof.

### Histone Deacetylase Inhibitors

The methods described herein in some embodiments comprise administration of nanoparticle compositions of mTOR inhibitors in combination with a histone deacetylase inhibitor. Histone deacetylase (HDAC) inhibitors have demonstrated significant clinical benefit as single agents in cutaneous and peripheral T cell lymphomas, and have received FDA approval for these indications.

Histone deacetylases are divided into 4 classes: class-I (HDAC1, 2, 3, 8), class-IIa (HDAC4, 5, 7, 9), class-IIb (HDAC6, 10), class-III (SIRT1-7), and class-IV (HDAC11). These classes differ in their subcellular localization (class-I HDACs are present in nucleus and class-II enzymes are cytoplasmic) and their intracellular targets. Although HDACs are typically associated with target histone proteins, recent studies reveal at least 3,600 acetylation sites on 1,750 non-histone proteins in cancer cells associated with various functions including gene expression, DNA replication and repair, cdl cycle progression, cytoskeletal reorganization, and protein chaperone activity. Clinical trials with non-selective HDAC inhibitors (HDACi) have shown efficacy, but are limited due to side effects, such as fatigue, diarrhea, and thrombocytopenia.

HDAC inhibitors include, but are not limited to, vorinostat (SAHA), panobinostat (LBH589), belinostat (PXD101, CAS 414864-00-9), tacedinaline (N-acetyldinaline, CI-994), givinostat (gavinostat, ITF2357), FRM-0334 (EVP-0334), resveratrol (SRT501), CUDC-101, quisinostat (JNJ-26481585), abexinostat (PCI-24781), dacinostat (LAQ824, NVP-LAQ824), valproic acid, 4-(dimethylamino)N-[6-(hydroxyamino)-6-oxohexyl]-benzamide (HDAC1 inhibitor), 4-Iodo suberoylanilide hydroxamic acid (HDAC1 and HDAC6 inhibitor), romidepsin (a cyclic tetrapeptide with HDAC inhibitory activity primarily towards class-I HDACs), 1-naphthohydroxamic acid (HDAC1 and HDAC6 inhibitor), HDAC inhibitors based on amino-benzamide biasing elements (e.g., mocetinostat (MGCD103) and entinostat (MS275), which are highly selective for HDAC1, 2 and 3), AN-9 (CAS 122110-53-6), APHA Compound 8 (CAS 676599-90-9), apicidin (CAS 183506-66-3), BML-210 (CAS 537034-17-6), salermide (CAS 1105698-15-4), suberoyl bis-hydroxamic acid (CAS 38937-66-5) (HDAC1 and HDAC3 inhibitor), butyrylhydroxamic acid (CAS 4312-91-8), CAY10603 (CAS 1045792-66-2) (HDAC6 inhibitor), CBHA (CAS 174664-65-4), ricolinostat (ACY1215, rocilinostat), trichostatin-A, WT-161, tubacin, and Merck60.

In some embodiments, the HDAC inhibitor is a nucleotide based or protein/peptide based inhibitor of an HDAC. For example, nucleotide based inhibitors of an HDAC can include, but are not limited to, short hairpin RNA (shRNA), RNA interference (RNAi), short interfering RNA (siRNA), microRNA (miRNA), locked nucleic acids (LNA), DNA, peptide-nucleic acids (PNA), morpholinos, and aptamers. In some embodiments, nucleotide based inhibitors are composed of at least one modified base. In some embodiments, nucleotide based inhibitors bind to the mRNA of an HDAC and decrease or inhibit its translation, or increase its degradation. In some embodiments, nucleotide based inhibitors decrease the expression (e.g., at the mRNA transcript and/or protein level) of an HDAC in cells and/or in a subject. In some embodiments, nucleotide based inhibitors bind to an HDAC and decrease its enzymatic activity

Protein or peptide based inhibitors of an HDAC can include but are not limited to peptides, recombinant proteins, and antibodies or fragments thereof. Protein or peptide based inhibitors can be composed of at least one non-natural amino acid. In some embodiments protein or peptide based inhibitors decrease the expression (e.g., at the mRNA transcript and/or protein level) of an HDAC in cells and/or in a subject. In some embodiments, protein or peptide based inhibitors bind to an HDAC and decrease its enzymatic activity.

Methods for identifying and/or generating nucleotide based or protein/peptide based inhibitors for a protein described herein are commonly known in the art.

In some embodiments, according to any of the methods described above, the histone deacetylase inhibitor may include, but is not limited to, vorinostat (SAHA), panobinostat (LBH589), belinostat (PXD101, CAS 414864-00-9), tacedinaline (N-acetyldinaline, CI-994), givinostat (gavinostat, ITF2357), FRM-0334 (EVP-0334), resveratrol (SRT501), CUDC-101, quisinostat (JNJ-26481585), abexinostat (PCI-24781), dacinostat (LAQ824, NVP-LAQ824), valproic acid, 4-(dimethylamino) N-[6-(hydroxyamino)-6-oxohexyl]-benzamide (HDAC1 inhibitor), 4-Iodo suberoylanilide hydroxamic acid (HDAC1 and HDAC6 inhibitor), romidepsin (a cyclic tetrapeptide with HDAC inhibitory activity primarily towards class-I HDACs), 1-naphthohydroxamic acid (HDAC1 and HDAC6 inhibitor), HDAC inhibitors based on amino-benzamide biasing elements (e.g., mocetinostat (MGCD103) and entinostat (MS275), which are highly selective for HDAC1, 2 and 3), AN-9 (CAS 122110-53-6), APHA Compound 8 (CAS 676599-90-9), apicidin (CAS 183506-66-3), BML-210 (CAS 537034-17-6), salermide (CAS 1105698-15-4), suberoyl bis-hydroxamic acid (CAS 38937-66-5) (HDAC1 and HDAC3 inhibitor), butyrylhydroxamic acid (CAS 4312-91-8), CAY10603 (CAS 1045792-66-2) (HDAC6 inhibitor), CBHA (CAS 174664-65-4), ricolinostat (ACY1215, rocilinostat), trichostatin-A, WT-161, tubacin, and Merck60.

Thus, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of vorinostat (SAHA), panobinostat (LBH589), belinostat (PXD101, CAS 414864-00-9), tacedinaline (N-acetyldinaline, CI-994), givinostat (gavinostat, ITF2357), FRM-0334 (EVP-0334), resveratrol (SRT501), CUDC-101, quisinostat (JNJ-26481585), abexinostat (PCI-24781), dacinostat (LAQ824, NVP-LAQ824), valproic acid, 4-(dimethylamino) N-[6-(hydroxyamino)-6-oxohexyl]-benzamide (HDAC1 inhibitor), 4-Iodo suberoylanilide hydroxamic acid (HDAC1 and HDAC6 inhibitor), romidepsin (a cyclic tetrapeptide with HDAC inhibitory activity primarily towards class-I HDACs), 1-naphthohydroxamic acid (HDAC1 and HDAC6 inhibitor), HDAC inhibitors based on amino-benzamide biasing elements (e.g., mocetinostat (MGCD103) and entinostat (MS275), which are highly selective for HDAC1, 2 and 3), AN-9 (CAS 122110-53-6), APHA Compound 8 (CAS 676599-90-9), apicidin (CAS 183506-66-3), BML-210 (CAS 537034-17-6), salermide (CAS 1105698-15-4), suberoyl bis-hydroxamic acid (CAS 38937-66-5) (HDAC1 and HDAC3 inhibitor), butyrylhydroxamic acid (CAS 4312-91-8), CAY10603 (CAS 1045792-66-2) (HDAC6 inhibitor), CBHA (CAS 174664-65-4), ricolinostat (ACY1215, rocilinostat), trichostatin-A, WT-161, tubacin, and Merck60. In some embodiments, the histone deacetylase inhibitor is romidepsin.

### Kinase Inhibitors

The methods described herein in some embodiments comprise administration of nanoparticle compositions of mTOR inhibitors in combination with a kinase inhibitor (such as a tyrosine kinase inhibitor). Kinase inhibitors have demonstrated significant clinical benefit as single agents for several indications, including non-small cell lung cancer, renal cell carcinoma, and chronic myeloid leukemia, and have received FDA approval for these indications.

A kinase is an enzyme that catalyzes the transfer of phosphate groups from high-energy, phosphate-donating molecules to specific substrates. Kinases are part of the larger family of phosphotransferases. The phosphorylation state of a molecule, whether it be a protein, lipid, or carbohydrate, can affect its activity, reactivity, and/or its ability to bind other molecules. Therefore, kinases are critical in metabolism, cell signaling, protein regulation, cellular transport, secretory processes, and many other cellular pathways.

Protein kinases act on proteins, phosphorylating them on serine, threonine, tyrosine, and/or histidine residues. Phosphorylation can modify the function of a protein in many ways. It can increase or decrease a protein's activity, stabilize it or mark it for destruction, localize it within a specific cellular compartment, and it can initiate or disrupt its interaction with other proteins. The protein kinases make up the majority of all kinases and are widely studied. These kinases, in conjunction with phosphatases, play a major role in protein and enzyme regulation as well as signaling in the cell.

“Kinase inhibitors,” as used herein, refer to molecules and pharmaceuticals, the administration of which to a subject results in the inhibition of a kinase. Examples of tyrosine kinase inhibitors include, but are not limited to, apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib.

In some embodiments, according to any of the methods described above, the kinase inhibitor may include, but is not limited to, apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is nilotinib. In some embodiments, the kinase inhibitor is sorafenib.

Thus, in some embodiments, there is provided a method of treating a hematological malignancy (such as lymphoma, leukemia, and myeloma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib. In some embodiments, the kinase inhibitor is nilotinib. In some embodiments, the kinase inhibitor is sorafenib.

### Cancer Vaccines

The methods described herein in some embodiments comprise administration of nanoparticle compositions of mTOR inhibitors in combination with a cancer vaccine (such as a vaccine prepared using autologous or allogeneic tumor cells or a TAA). Cancer vaccines have demonstrated significant clinical benefit in therapies for several hematological malignancies, including acute myeloid leukemia and follicular lymphoma.

A cancer vaccine is a form of active immunotherapy that increases the ability of an individual's immune system to respond to a TAA and mount an immune response to eliminate malignant cells (Melero, I. et al. (2014). Nature reviews Clinical oncology, 11(9), 509-524). Cancer vaccines may be designed to target multiple, undefined antigens, or to specifically target a given antigen or group of antigens. Polyvalent vaccines can be prepared from autologous or allogeneic cells, such as from whole tumor cells or from dendritic cells that have been fused with tumor cells, transfected with DNA or RNA derived from a tumor, or loaded with lysate from tumor cells. Antigen-specific vaccines can be prepared from a single antigen, including short peptides with narrow epitope specificity or long peptides having multiple epitopes, or from a mixture of several different antigens.

The immunogenicity of antigens in a cancer vaccine can be increased in several ways, such as by combining the antigen with one or more adjuvants. Adjuvants can be selected to elicit a desired immune response for cancer immunotherapy, such as activation of type 1 T helper cells (T_{H}1) and cytotoxic T lymphocytes (CTLs). Adjuvants useful for cancer vaccines include, for example, alum (such as aluminum hydroxide or phosphate), microbes and microbial derivatives (such as the bacterium Bacillus Calmette-Guérin, CpG, Detox B, monophosphoryl lipid A, and poly I:C), keyhole limpet hemocyanin (KLH), oil emulsions or surfactants (such as AS02, AS03, MF59, Montanide ISA-51™, and QS21), particulates (such as AS04, polylactide co-glycolide, and virosomes), viral vectors (such as adenovirus, vaccinia, and fowlpox), delta innulin based synthetic polysaccharide, imidzaquinolines, saponins, flagellin, and natural or synthetic cytokines (such as IL-2, IL-12, IFN-α, and GM-CSF). See, for example, Banday, A. H. et al. (2015). Immunopharmacology and immunotoxicology, 37(1), 1-11 and Melero, I. et al., supra. Antigens and adjuvants can also be packaged in immunogenic delivery vehicles to increase cancer vaccine potency. Such delivery vehicles include, but are not limited to, liposomal microspheres, recombinant viral vectors, and cultured mature dendritic cells Immunogenicity can also be increased by using a prime/boost strategy, where the immune system is primed with a first cancer vaccine targeting an antigen then boosted with a second cancer vaccine targeting the same antigen but in a different vector.

A cancer vaccine may include any molecules and pharmaceuticals, the administration of which to a subject results in an increase in the ability of the subject's immune system to mount an immune response against at least one tumor-associated antigen. Examples of cancer vaccines include, but are not limited to, polyvalent vaccines prepared from autologous tumor cells, polyvalent vaccines prepared from allogeneic tumor cells, and antigen-specific vaccines prepared from at least one tumor-associated antigen. Antigen-specific vaccines can comprise the at least one tumor-associated antigen, fragments thereof, or nucleic acids (such as recombinant viral vectors) encoding the at least one tumor-associated antigen or fragments thereof.

In some embodiments, according to any of the methods described above, the cancer vaccine may include, but is not limited to, a vaccine prepared using autologous tumor cells, a vaccine prepared using allogeneic tumor cells, and a vaccine prepared using at least one tumor-associated antigen (TAA). In some embodiments, the TAA is selected, for example, from the group consisting of heat shock proteins, melanocyte antigen gp100, MAGE antigens, BAGE, GAGE, NY-ESO-1, Melan-A, PSA, HER2, hTERT, p53, survivin, KRAS, WT1, alphafetoprotein (AFP), carcinoembryonic antigen (CEA), CA-125, GM2, MUC-1, epithelial tumor antigen (ETA), tyrosinase, and Trp-2. In some embodiments, the TAA is a neo-antigen, such as bcr-abl or a mutated form of a protein selected from the group consisting of β-catenin, HSP70-2, CDK4, MUM1, CTNNB1, CDC27, TRAPPC1, TPI, ASCC3, HHAT, FN1, OS-9, PTPRK, CDKN2A, HLA-A11, GAS7, GAPDH, SIRT2, GPNMB, SNRP116, RBAF600, SNRPD1, Prdx5, CLPP, PPP1R3B, EF2, ACTN4, ME1, NF-YC, HLA-A2, HSP70-2, KIAA1440, and CASP8 (for examples of identifying neoantigens see Gubin, M. M. et al. (2015). The Journal of clinical investigation, 125(9), 3413-3421; Lu, Y. C., & Robbins, P. F. (2016, February). Seminars in immunology. 28(1): 22-27; and Schumacher, T. N., & Schreiber, R. D. (2015). Science, 348(6230), 69-74). In some embodiments, the TAA is a polypeptide derived from a virus implicated in human cancer, such as Human Papilloma Viruses (HPV), Hepatitis Viruses (HBV and HCV), Human T-Lymphotropic Virus (HTLV), Merkel cell polyomavirus, Epstein-Barr Virus (EBV), and Kaposi's Sarcoma-associated Herpesvirus (KSHV).

Suitable cancer vaccines include, for example, PVX-410 Multi-Peptide Vaccine.

### Articles of Manufacture and Kits

In some embodiments of the invention, there is provided an article of manufacture containing materials useful for the treatment of a hematological malignancy comprising an mTOR inhibitor nanoparticle composition (such as sirolimus/albumin nanoparticle composition) and a second therapeutic agent. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is a) a nanoparticle formulation of an mTOR inhibitor; or b) a second therapeutic agent. The label or package insert indicates that the composition is used for treating the particular condition in an individual. The label or package insert will further comprise instructions for administering the composition to the individual. Articles of manufacture and kits comprising combination therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a hematological malignancy (such as lymphoma, leukemia, and myeloma).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a hematological malignancy (such as lymphoma, leukemia, and myeloma). Kits of the invention include one or more containers comprising an mTOR inhibitor nanoparticle composition (such as sirolimus/albumin nanoparticle composition) (or unit dosage form and/or article of manufacture), and in some embodiments, further comprise a second therapeutic agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an mTOR inhibitor nanoparticle composition (such as sirolimus/albumin nanoparticle composition). In some embodiments, the kit comprises a) a composition comprising an mTOR inhibitor nanoparticle composition (such as sirolimus/albumin nanoparticle composition), and b) a second therapeutic agent. In some embodiments, the kit comprises a) a composition comprising an mTOR inhibitor nanoparticle composition (such as sirolimus/albumin nanoparticle composition), and b) instructions for administering the mTOR inhibitor nanoparticle composition in combination with a second therapeutic agent to an individual for treatment of a hematological malignancy, such as multiple myeloma, mantle cell lymphoma, T cell lymphoma, chronic myeloid leukemia, and acute myeloid leukemia. In some embodiments, the kit comprises a) a composition comprising an mTOR inhibitor nanoparticle composition (such as sirolimus/albumin nanoparticle composition), b) a second therapeutic agent, and c) instructions for administering the mTOR inhibitor nanoparticle composition and the second therapeutic agent to an individual for treatment of a hematological malignancy, such as multiple myeloma, mantle cell lymphoma, T cell lymphoma, chronic myeloid leukemia, and acute myeloid leukemia. The mTOR inhibitor nanoparticle composition (such as sirolimus/albumin nanoparticle composition) and the second therapeutic agent can be present in separate containers or in a single container. For example, the kit may comprise one distinct composition or two or more compositions wherein one composition comprises an mTOR inhibitor nanoparticle composition (such as sirolimus/albumin nanoparticle composition) and another composition comprises the second therapeutic agent.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the mTOR inhibitor nanoparticle composition (such as sirolimus/albumin nanoparticle composition) and the second therapeutic agent generally include information as to dosage, dosing schedule, and route of administration for the intended treatment. The containers may be unit doses, bulk packages (e.g., multi-dose packages) or sub-unit doses. For example, kits may be provided that contain sufficient dosages of an mTOR inhibitor nanoparticle composition (such as sirolimus/albumin nanoparticle composition) and a second therapeutic agent as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the mTOR inhibitor nanoparticle composition (such as sirolimus/albumin nanoparticle composition) and the second therapeutic agent and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

### Embodiment 1

A method of treating a hematological malignancy in an individual, comprising administering to the individual: a) an effective amount of a composition comprising nanoparticles comprising an mTOR inhibitor and an albumin, and b) an effective amount of a second therapeutic agent, wherein the second therapeutic agent is selected from the group consisting of an immunomodulator, a histone deacetylase inhibitor, a kinase inhibitor, and a cancer vaccine.

### Embodiment 2

In some further embodiments of embodiment 1, the hematological malignancy is multiple myeloma, mantle cell lymphoma, T cell lymphoma, chronic myeloid leukemia, or acute myeloid leukemia.

### Embodiment 3

In some further embodiments of embodiment 1 or 2, the hematological malignancy is relapsed or refractory to a standard therapy for the hematological malignancy.

### Embodiment 4

In some further embodiments of any one of embodiments 1-3, the amount of the mTOR inhibitor in the mTOR inhibitor nanoparticle composition is from about 10 mg/m² to about 150 mg/m².

### Embodiment 5

In some further embodiments of embodiment 4, the amount of the mTOR inhibitor in the mTOR inhibitor nanoparticle composition is about 45 mg/m² to about 100 mg/m².

### Embodiment 6

In some further embodiments of embodiment 4, the amount of the mTOR inhibitor in the mTOR inhibitor nanoparticle composition is about 75 mg/m² to about 100 mg/m².

### Embodiment 7

In some further embodiments of any one of embodiments 1-6, the mTOR inhibitor nanoparticle composition is administered weekly.

### Embodiment 8

In some further embodiments of any one of embodiments 1-6, the mTOR inhibitor nanoparticle composition is administered 3 out of every 4 weeks.

### Embodiment 9

In some further embodiments of any one of embodiments 1-8, the mTOR inhibitor nanoparticle composition and the second therapeutic agent are administered sequentially to the individual.

### Embodiment 10

In some further embodiments of any one of embodiments 1-8, the mTOR inhibitor nanoparticle composition and the second therapeutic agent are administered simultaneously to the individual.

### Embodiment 11

In some further embodiments of any one of embodiments 1-10, the mTOR inhibitor is a limus drug.

### Embodiment 12

In some further embodiments of embodiment 11, the limus drug is sirolimus.

### Embodiment 13

In some further embodiments of any one of embodiments 1-12, the average diameter of the nanoparticles in the composition is no greater than about 150 nm.

### Embodiment 14

In some further embodiments of embodiment 13, the average diameter of the nanoparticles in the composition is no greater than about 120 nm.

### Embodiment 15

In some further embodiments of any one of embodiments 1-14, the weight ratio of the albumin to the mTOR inhibitor in the nanoparticle composition is no greater than about 9:1.

### Embodiment 16

In some further embodiments of any one of embodiments 1-15, the nanoparticles comprise the mTOR inhibitor associated with the albumin.

### Embodiment 17

In some further embodiments of embodiment 16, the nanoparticles comprise the mTOR inhibitor coated with the albumin.

### Embodiment 18

In some further embodiments of any one of embodiments 1-17, the mTOR inhibitor nanoparticle composition is administered intravenously, intraarterially, intraperitoneally, intravesicularly, subcutaneously, intrathecally, intrapulmonarily, intramuscularly, intratracheally, intraocularly, transdermally, orally, or by inhalation.

### Embodiment 19

In some further embodiments of embodiment 18, the mTOR inhibitor nanoparticle composition is administered intravenously.

### Embodiment 20

In some further embodiments of any one of embodiments 1-19, the individual is human.

### Embodiment 21

In some further embodiments of any one of embodiments 1-20, the method further comprises selecting the individual for treatment based on the presence of at least one mTOR-activating aberration.

### Embodiment 22

In some further embodiments of embodiment 21, the mTOR-activating aberration comprises a mutation in an mTOR-associated gene.

### Embodiment 23

In some further embodiments of embodiment 21 or 22, the mTOR-activating aberration is in at least one mTOR-associated gene selected from the group consisting of AKT1, FLT-3, MTOR, PIK3CA, TSC1, TSC2, RHEB, STK11, NF1, NF2, KRAS, NRAS and PTEN.

### Embodiment 24

In some further embodiments of any one of embodiments 1-23, the second therapeutic agent is an immunomodulator.

### Embodiment 25

In some further embodiments of embodiment 24, the immunomodulator is an IMiDs®.

### Embodiment 26

In some further embodiments of embodiment 24, the immunomodulator is an immune checkpoint inhibitor.

### Embodiment 27

In some further embodiments of embodiment 24, the immunomodulator is selected from the group consisting of pomalidomide and lenalidomide.

### Embodiment 28

In some further embodiments of embodiment 27, the hematological malignancy is multiple myeloma and the second therapeutic agent is pomalidomide.

### Embodiment 29

In some further embodiments of embodiment 27, the hematological malignancy is mantle cell lymphoma and the second therapeutic agent is lenalidomide.

### Embodiment 30

In some further embodiments of any one of embodiments 24-29, the method further comprises selecting the individual for treatment based on the presence of at least one biomarker indicative of favorable response to treatment with an immunomodulator.

### Embodiment 31

In some further embodiments of embodiment 30, the at least one biomarker comprises a mutation in an immunomodulator-associated gene.

### Embodiment 32

In some further embodiments of any one of embodiments 1-23, the second therapeutic agent is a histone deacetylase inhibitor.

### Embodiment 33

In some further embodiments of embodiment 32, the histone deacetylase inhibitor is selected from the group consisting of romidepsin, panobinostat, ricolinostat, and belinostat.

### Embodiment 34

In some further embodiments of embodiment 33, the hematological malignancy is T cell lymphoma and the histone deacetylase inhibitor is romidepsin.

### Embodiment 35

In some further embodiments of any one of embodiments 32-34, the method further comprises selecting the individual for treatment based on the presence of at least one biomarker indicative of favorable response to treatment with a histone deacetylase inhibitor (HDACi).

### Embodiment 36

In some further embodiments of embodiment 35, the at least one biomarker comprises a mutation in an HDAC-associated gene.

### Embodiment 37

In some further embodiments of any one of embodiments 1-23, the second therapeutic agent is a kinase inhibitor.

### Embodiment 38

In some further embodiments of embodiment 37, the kinase inhibitor is selected from the group consisting of nilotinib and sorafenib.

### Embodiment 39

In some further embodiments of embodiment 38, the hematological malignancy is chronic myeloid leukemia and the kinase inhibitor is nilotinib.

### Embodiment 40

In some further embodiments of embodiment 38, the hematological malignancy is acute myeloid leukemia and the kinase inhibitor is sorafenib.

### Embodiment 41

In some further embodiments of any one of embodiments 37-40, the method further comprises selecting the individual for treatment based on the presence of at least one biomarker indicative of favorable response to treatment with a kinase inhibitor.

### Embodiment 42

In some further embodiments of any one of embodiments 1-23, the second therapeutic agent is a cancer vaccine.

### Embodiment 43

In some further embodiments of embodiment 42, the cancer vaccine is selected from the group consisting of a vaccine prepared from autologous tumor cells, a vaccine prepared from allogeneic tumor cells, and a vaccine prepared from at least one tumor-associated antigen.

### Embodiment 44

In some further embodiments of embodiment 42 or 43, the method further comprises selecting the individual for treatment based on the presence of at least one biomarker indicative of favorable response to treatment with a cancer vaccine.

### Embodiment 45

In some further embodiments of embodiment 44, the at least one biomarker comprises a mutation in a cancer vaccine-associated gene.

### Embodiment 46

In some further embodiments of any one of embodiments 42-45, the hematological malignancy is selected from the group consisting of multiple myeloma, chronic myeloid leukemia, acute myeloid leukemia, mantle cell lymphoma, and T cell lymphoma.

### EXAMPLES

### Example 1: Phase Ib/II Study with Patients Receiving ABI-009 Treatment in Combination with Standard Therapies for Relapsed/Refractory Multiple Myeloma

A multicenter, open-label phase Ib/II clinical trial is designed to evaluate the mTOR inhibitor ABI-009 (nab-sirolimus) in combination with selected anti-cancer drugs in patients with different relapsed/refractory hematological malignancies. The primary goals of the study are to evaluate the safety and tolerability of ABI-009 in different independent combinations in patients with advanced hematologic malignancies, to characterize the dose limiting toxicities (DLTs) and overall safety profile of escalated dose levels of ABI-009 and the associated dose schedule for each combination, and to determine the maximum tolerated dose (MTD) of ABI-009 for each combination. The secondary goals of the study are to investigate the efficacy of the mTOR inhibitor ABI-009 in combination with standard therapies in patients with hematologic malignancies (relapsed/refractory multiple myeloma, T-cell lymphoma, mantle cell lymphoma, chronic myeloid leukemia or acute myeloid leukemia) potentially sensitive to mTOR inhibition, and to evaluate the pharmacokinetics (PK) of ABI-009 in combination with other drugs. Exploratory objectives of the study include evaluating the pharmacodynamic effects with relation to safety and/or efficacy endpoints, exploring PK/pharmacodynamic relationships for safety and/or efficacy endpoints, exploring the predictive role of several tumor biomarkers (including, but not limited to, PI3K, mTOR, FLT-3ITD, AKT, KRAS, and NRAS) on clinical responsiveness, and investigating the effects of genetic variation in drug metabolism genes, cancer genes, and drug target genes on subject response to ABI-009.

This study is conducted in 2 parts: part 1—phase Ib dose escalation; and part 2—2 stage phase II study for each combination. Approximately 117 patients are enrolled in the study. In each part of the study, subjects are enrolled in parallel into one of six different independent arms.

In part 1, dose escalation, approximately 72 patients are enrolled into 6 independent arms with the following pre-specified nominal doses (additional doses are also evaluated if required or supported by emerging data):

| Arm | Cohort | ABI-009 Dose/m² | |
|---|---|---|---|
| 1 | 1 | 45 | mg |
| ABI-009 + pomalidomide | 2 | 75 | mg |
| (Multiple Myeloma) | 3 | 100 | mg |
| 2 | 1 | 45 | mg |
| ABI-009 + Lenalidomide | 2 | 75 | mg |
| (Mantle Cell Lymphoma) | 3 | 100 | mg |
| 3 | 1 | 45 | mg |
| ABI-009 + Romidepsin | 2 | 75 | mg |
| (Multiple Myeloma) | 3 | 100 | mg |
| 4 | 1 | 45 | mg |
| | 2 | 75 | mg |
| | 3 | 100 | mg |
| 5 | 1 | 45 | mg |
| ABI-009 + Nilotinib | 2 | 75 | mg |
| (Chronic Myeloid Leukemia) | 3 | 100 | mg |
| 6 | 1 | 45 | mg |
| ABI-009 + Sorafenib | 2 | 75 | mg |
| (Acute Myeloid Leukemia) | 3 | 100 | mg |

### Part 1—Dose Escalation

Patients in the dose escalation part of the study, aimed at determining an ABI-009 MTD when combined with selected anti-cancer drugs, receive a fixed dose of the selected combination drug(s), per standard of care. Safety, tolerability, PK and pharmacodynamics are evaluated for each combination.

ABI-009 is administered IV, weekly 3 weeks on and 1 week off, with planned nominal ABI-009 doses of 45, 75, and 100 mg/m². Additional doses may be explored. The first ABI-009 MTD to be estimated for cohort 1 of each arm is with full recommended doses of the selected anti-cancer drug(s). Other ABI-009 schedules can be explored based on emerging clinical data.

Dose escalation decisions consider the incidence of dose limiting toxicities (DLTs) among DLT-evaluable subjects that occur during cycle 1 (28-day period). A cohort of 3 to 4 DLT-evaluable subjects are enrolled per dose level.

A Toxicity Probability Interval (TPI) Bayesian model design is used to estimate the ABI-009 MTD in combination with the selected anti-cancer drug(s) for each arm where “toxicity” refers to DLT (Neuenschwander et al., 2008).

For each arm, the DLRM may consider part 1 complete if 1 of the following rules is met: i) the highest planned dose level is evaluated with no DLTs in cycle 1 at any dose level (if this occurs, the maximum administered dose may be used for part 2); ii) the Bayesian model recommends the same dose >2 times (not necessarily sequentially); or iii) a total of 12 DLT-evaluable subjects have been enrolled.

### Part 2—Phase II Study

Up to 3 arms for a total of 45 patients are enrolled in the 2 stage phase II study to confirm safety and tolerability and to assess clinical activity.

Arms are selected for phase II based on the safety and efficacy profile of the dose escalation part of the study. An arm is not selected for dose expansion unless it has an acceptable safety profile and at least 2 proven clinical efficacy events are observed (TBC). For each arm that participates in the phase II, the dose is evaluated based upon results from the dose escalation phase of the corresponding arm. Subjects that come off study prior to completing 3 months on study due to reasons other than disease progression may be replaced. On completion of the phase II, a final estimate of the MTD is determined from the Bayesian model utilizing all part 1 and 2 DLT-evaluable subjects.

Based on emerging clinical data, combination arms can be stopped. On the other hand, based on new synergy data reported in the literature and/or on current standard of care, additional or different combinations may be explored in part 1 or part 2.

### Study Population

A patient is eligible for inclusion in this study only if all of the following criteria are met: i) age>18 years old; ii) adequate organ and marrow function defined as: a) absolute neutrophil count>1.0×10⁹/L; b) platelet count>75×10⁹/L; and c) hemoglobin >9 g/dL (transfusions are permitted but the most recent transfusion must have been ≥7 days prior to obtaining the screening hemoglobin); iii) estimated glomerular filtration rate based on MDRD (Modification of Diet in Renal Disease) calculation ≥45 ml/min/1.73 m²; iv) adequate hepatic laboratory assessments, as follows: a) AST <2.5×ULN (if liver metastases are present, ≤5×ULN); b) ALT <2.5×ULN (if liver metastases are present, ≤5×ULN); c) alkaline phosphatase <2.0×ULN (if liver or bone metastases are present, <3.0×ULN); and d) total bilirubin <1.5×ULN (<2.0×ULN for subjects with documented Gilbert's syndrome or <3.0×ULN for subjects for whom the indirect bilirubin level suggests an extrahepatic source of elevation); v) Eastern Cooperative Oncology Group performance status 0-2; vi) life expectancy of at least 12 weeks; vii) disease free of prior malignancies for greater than or equal to 1 year with exception of currently treated basal cell, squamous cell carcinoma of the skin, or carcinoma “in situ” of the cervix or breast; viii) fasting serum cholesterol ≤300 mg/dL OR ≤7.75 mmol/L AND fasting triglycerides ≤2.5×ULN; ix) must agree to receive counseling related to teratogenic and other risks; x) understand and voluntarily sign an informed consent form; xi) able to adhere to the study visit schedule and other protocol requirements; xii) must agree to follow pregnancy precautions as required by the protocol; and xiii) must agree not to donate blood or semen.

A patient is eligible for inclusion in arms 1 and 3 of this study only if all of the following criteria are met: i) pathologically documented, definitively diagnosed, multiple myeloma relapsed or progressive disease after at least 1 but no more than 3 prior therapeutic treatments or regimens for multiple myeloma; ii) prior therapeutic treatment or regimens may have included bortezomib, lenalidomide, and/or thalidomide, among other agents; iii) must be willing and able to undergo bone marrow aspirate per protocol (with or without bone marrow biopsy per institutional guidelines); iv) measurable disease, as indicated by one or more of the following: a) serum M-protein ≥0.5 g/dl; b) urine M-protein ≥200 mg/24 hour or abnormal free light chain (FLC) ratio (if Serum Protein Electrophoresis is felt to be unreliable for routine M-protein measurement, particularly for patients with IgA MM, then quantitative immunoglobulin levels can be accepted); or c) serum free light chain (sFLC) assay: Involved FLC assay ≥10 mg/dL (≥100 mg/L) and an abnormal sFLC ratio (<0.26 or >1.65) as per the IMWG criteria; v) measureable plasmacytoma (prior biopsy is acceptable); vi) oligo or non-secretory myeloma subjects may be included if there is measurable plasmacytosis in the bone marrow biopsy or measurable extramedullary disease; and vii) prior to enrollment, evidence of myeloma progression/relapse must be provided, with start and stop dates of the most recent treatment regimen, as well as best tumor response to all prior treatment regimens.

A patient is eligible for inclusion in arm 2 of this study only if all of the following criteria are met: i) histopathologically confirmed MCL, relapsed and/or refractory to standard chemotherapy; and ii) two-dimensional measurable nodal lesion or ex-nodal lesion [>1.5 cm in greatest transverse diameter by computerized tomography (CT) scan].

A patient is eligible for inclusion in arm 4 of this study only if all of the following criteria are met: i) histopathologically confirmed MCL, relapsed and/or refractory to standard chemotherapy; ii) must have relapsed or progressed after at least two prior systemic cytotoxic chemotherapy; and iii) two-dimensional measurable nodal lesion or ex-nodal lesion [>1.5 cm in greatest transverse diameter by computerized tomography (CT) scan].

A patient is eligible for inclusion in arm 5 of this study only if all of the following criteria are met: i) BCR-ABL-positive CML in CP who had failed therapy with at least the standard dose imatinib (i.e., ≥400 mg daily). Imatinib failure is defined as: a) inability to achieve or loss of CHR after 3 months of imatinib; b) failure to achieve or loss of at least a minimal cytogenetic response after 6 months of imatinib; or c) failure to achieve or loss of a MCyR after 12 months of imatinib.

A patient is eligible for inclusion in arm 6 of this study only if all of the following criteria are met: i) pathologically-documented, definitively-diagnosed FLT3-ITD AML that is relapsed or refractory to standard treatment, for which no standard therapy is available or the subject refuses standard therapy; and ii) no more than 2 lines of prior therapy (a line of therapy is defined as a treatment course of therapy, which may include bone marrow transplant or successive courses of chemotherapy, that occurs without evidence of disease progression).

A patient is ineligible for inclusion in this study if any of the following criteria are met: 1) prior mTOR inhibitor; ii) prior history of cancer, other than MM, MCL, T Cell Lymphoma, CML or AML, unless the subject has been free of the disease for ≥1 year. (Basal cell carcinoma of the skin, carcinoma in situ of the cervix, or stage T1a or T1b prostate cancer is allowed); iii) renal insufficiency (CrC1<40 mL/min by Cockroft-Gault method); iv) uncontrolled hyperthyroidism or hypothyroidism; v) history of interstitial lung disease or pneumonitis; vi) grade ≥2 neuropathy; vii) history of deep venous thrombosis (DVT) or pulmonary embolus (PE) within past 3 years; viii) significant active cardiac disease within the past 6 months; ix) known HIV infection; known Hepatitis C infection or active Hepatitis B infection; x) any serious medical condition, laboratory abnormality, or psychiatric illness that would prevent the subject from signing the informed consent form; xi) any condition, including the presence of laboratory abnormalities; xii) use of any other anti-cancer drug or therapy, including experimental, within 30 days of enrollment; xiii) known positive for HIV or infectious hepatitis, type A, B or C; xiv) pregnant or breastfeeding females; or xv) concurrent use of other anti-cancer agents or treatments.

A patient is ineligible for inclusion in arms 1 or 3 this study if any of the following criteria are met: i) history of allogeneic stem cell transplant with active graft-versus-host disease requiring immunosuppressive therapy, and/or peripheral grade ≥2 are excluded from the trial; ii) prior treatment with pomalidomide (Arm1) or HDAC inhibitor (Arm3); iii) non-secretory or hyposecretory multiple myeloma, defined as <0.5 g/dL M-protein in serum, <200 mg/24 hour urine M-protein, or disease only measured by sFLC; iv) subjects who never achieved at least a durable minimal response (≥25% reduction in M-protein for at least 6 weeks) on any prior therapy; v) corticosteroid therapy in a dose equivalent to dexamethasone ≥4 mg/day or prednisone ≥30 mg/day within 3 weeks prior to study day 1; vi) use of any other experimental drug or therapy within 28 days of study day 1; vii) POEMS syndrome (polyneuropathy, organomegaly, endocrinopathy, monoclonal protein, and skin changes); or viii) plasma cell leukemia or Waldenstrom's macroglobulinemia.

A patient is ineligible for inclusion in arm 2 this study if any of the following criteria are met: i) history of allogeneic stem cell transplant with active graft-versus-host disease requiring immunosuppressive therapy, and/or peripheral grade ≥2 are excluded from the trial; or ii) prior treatment with lenalidomide.

A patient is ineligible for inclusion in arm 4 this study if any of the following criteria are met: i) patients who are candidates for high dose chemotherapy and stem cell transplantation and have not yet undergone stem cell transplantation should not be enrolled; or ii) prior treatment with HDAC inhibitor.

A patient is ineligible for inclusion in arm 5 this study if any of the following criteria are met: i) prior treatment with nilotinib.

A patient is ineligible for inclusion in arm 6 this study if any of the following criteria are met: i) acute promyelocytic leukemia or active central nervous system leukemia; ii) any prior bone marrow transplant within 8 weeks of day 1 for which the subject is receiving systemic immunosuppression or shows signs of Graft-versus-Host Disease; or iii) history risk of retinal vein occlusion (RVO).

### Treatment

The investigational product used in this study refers to: ABI-009 given intravenously (IV) on days 1, 8, and 15 of a 28 day cycle, with a starting dose of 45 mg/m² and a planned dose escalation of 45, 75, and 100 mg/m². The part 1 dose escalation is aimed at determining an ABI-009 MTD with a fixed dose, per standard of care, of the combination drug(s).

The fixed starting dose level for the combination drug(s) are as follows: i) pomalidomide, 4 mg taken orally on days 1-21 of repeated 28-day cycles+low dose dexamethasone (40 mg weekly); ii) lenalidomide, 25 mg once daily orally on days 1-21 of repeated 28-day cycles; iii) romidepsin, 14 mg/m² IV over a 4-hour period on days 1, 8 and 15 of repeated 28-day cycles; iv) nilotinib, 300 mg orally BID; and v) sorafenib, 400 mg orally BID.

Patients continue therapy until disease progression. The End of Trial is defined as either the date of the last visit of the last patient to complete the study, or the date of receipt of the last data point from the last patient that is required for primary, secondary, and/or exploratory analysis, as pre-specified in the protocol.

### Multiple Myeloma

For patients with multiple myeloma, the IMWG response criteria is used for efficacy assessment with revisions and improvements that include the addition of FLC response and progression criteria for subjects without measurement disease, modification of the definition for disease progression for subjects with CR, and addition of very good partial response (VGPR) and stringent response categories. Bone marrow confirmation is required for coding CR (Rajkumar et al, 2011; Durie et al, 2006). For subjects without a history of extramedullary disease, assessment by physical examination at screening is acceptable. Plasmacytoma evaluation is repeated during treatment only to confirm a response of PR or better, to confirm PD, or if clinically indicated. If clinically indicated, due to history of extramedullary disease, the same technique (CT scan or MRI) must be employed for each measurement. The following examinations are performed for efficacy assessment: i) serum protein electrophoresis (SPEP) and urine protein electrophoresis (UPEP) with 24-hour urine collection must be done at screening (thereafter, SPEP is done pre-dose at each cycle; UPEP at each cycle is required only if screening UPEP shows measureable M-protein in the urine); ii) quantification of serum immunoglobulins; iii) sFLC assay and ratio only required if SPEP or UPEP results are undetectable; and iv) serum β-2 microglobulin and lactate dehydrogenase done pre-dose at each cycle.

### Mantle Cell and T Cell Lymphoma

Evaluation of efficacy is based on Revised Response Criteria for Malignant Lymphoma (Cheson B D et al, 2007) and PET, CT, or MRI scans with contrast are acquired at baseline, 4 weeks after cycle 1 day 1, 8 weeks after cycle 1 day 1, and every 8 weeks thereafter until disease progression. In addition, objective responses (CR or PR by RECIST 1.1) are confirmed by consecutive repeat scan performed no less than 28 days after the criteria for response are first met. Scans are acquired with slice thickness of 5 mm or less. Baseline imaging studies are performed within 4 weeks prior to study day 1, although it is recommended that they be performed as close to the day of enrollment as possible.

### Chronic Myeloid Leukemia

Efficacy of the treatment is evaluated by complete hematological response rate, hematological response survival curve analysis, and white blood cell (WBC) count after each month of treatment.

### Acute Myeloid Leukemia

Disease response assessments are based upon review of cytogenetics, bone marrow aspirates, and peripheral blood count. Refer to revised International Working Group (IWG) response criteria. Complete response/complete recovery with incomplete count recovery (CRi) is established from bone marrow sample assessment supplemented with neutrophil, platelet, and peripheral blast counts.

In case of transplantation, a CR or CRi is confirmed within 4 weeks prior to transplantation.

### Safety

Safety and tolerability are monitored through continuous reporting of adverse events (AEs), AEs of special interest (identified based on previous experience in a similar population), laboratory abnormalities, and incidence of patients experiencing dose modifications, dose delay/dose not given, dose interruptions, and/or premature discontinuation of investigational product due to an AE. All AEs are recorded by the investigator from the time the subject signs informed consent until 28 days after the last dose of investigational product and those serious adverse events (SAEs) made known to the investigator at any time thereafter that are suspected of being related to investigational product. Toxicities are graded by National Cancer Institute (NCI) Common Terminology Criteria for Adverse Events (CTCAE) v4.0.

Physical examination (source documented only), vital sign, laboratory assessments (e.g., serum chemistry, hematology), and ECOG performance status are monitored. All SAEs (regardless of relationship to investigational product) are followed until resolution. Laboratory analysis is performed as per study schedule.

### Statistical Methods

In the phase Ib dose escalation part of the study, up to 12 patients are enrolled per arm, using the 3+3 dose escalation rule.

In the phase II study, 3 arms out of 6 are selected and explored. In each arm, the adaptive stage 2 design is used to determine efficacy of the most optimal doses of the combination regimens found in phase I. In stage 1, an initial cohort of patients are enrolled (n=24). At least 4 responses are required in stage 1 to enroll an additional 23 patient in stage 2. If the predefined futility criteria (<4 responses in stage 1) is not met, the respective arms will be expanded in Stage 2.

Sample size estimated based on a 2-stage design to test the null hypothesis of a response rate of P≤10% vs an alternative hypothesis of a response rate of P>10%. Using a 1-sided type I error rate of 0.05 and 80% power, 47 evaluable patients are required for the study. At least 4 responses are required in stage 1 (n=24) to enroll an additional 23 patients in stage 2, and at least 9 of 47 patients at the end of stage 2 are needed to reject the null hypothesis. Additional statistical tests are performed with a 2-sided significance level of 0.05. Point estimates and exact 95% confidence intervals are calculated for response rates, and Kaplan-Meier estimates are used to summarize PFS and OS.

### Example 2: Treatment of Hematological Malignancies with the Combination of nab-Sirolimus and Anti-CD38 Antibody

Mouse models of hematological malignancies are treated with the combination of ABI-009 and anti-CD38 antibody. A hematological malignancy cell line, such as human multiple myeloma cell line NCI-H929, is cultured, for example, in RPMI-1640 medium supplemented with 10% FBS, 2 mmol/L glutamine, and 1% penicillin-streptomycin at 37° C. with 5% CO₂. Mice, such as female CB.17 SCID mice, are inoculated, for example, subcutaneously with at least 1×10⁶ NCI-H929 cells (such as subcutaneously with 1×10⁷ NCI-H929 cells).

Treatment starts, for example, when tumors grow to an average volume of at least 50 mm³ (such as when tumors grow to an average volume of about 100 mm³). Mice are divided, for example, into at least one experimental group treated, for example, concurrently with the combination of ABI-009 and anti-human CD38 antibody, and one control group that receives no treatment or mock treatment. ABI-009 is administered, for example, intravenously (IV) at a dose of at least 5 mg/kg twice a week (such as IV at a dose of about 7.5 mg/kg twice a week). Anti-CD38 antibody is administered, for example, intraperitoneally (IP) at a dose of at least 5 mg/kg twice weekly for 3 weeks (such as IP at a dose of 10 mg/kg twice weekly for 3 weeks). The animals in each group are monitored, for example, for tumor volume, adverse response, histopathology of tumor, body weight and general health condition (eating, walking, daily activities).

### Example 3: Treatment of Hematological Malignancies with the Combination of nab-Sirolimus and Anti-PD-1 Antibody

Immunocompetent mice bearing syngeneic tumors are treated with the combination of ABI-009 and anti-PD-1 antibody (such as clone RMP1-14 from Bio X Cell, West Lebanon, N.H., USA). A hematological malignancy cell line, such as human multiple myeloma cell line NCI-H929, is cultured, for example, in RPMI-1640 medium supplemented with 10% FBS, 2 mmol/L glutamine, and 1% penicillin-streptomycin at 37° C. with 5% CO₂. Mice, such as female CB.17 SCID mice, are inoculated, for example, subcutaneously with at least 1×10⁶ NCI-H929 cells (such as subcutaneously with 1×10⁷ NCI-H929 cells).

Treatment starts when tumors grow, for example, to an average volume of 100 mm³. Mice are divided, for example, into at least one experimental group treated with the combination of ABI-009 and anti-PD-1 antibody, and one control group that receives no treatment or mock treatment. ABI-009 is administered, for example, intravenously (IV) at 5 mg/kg 3 times a week. Anti-PD1 antibody is administered, for example, intraperitoneally (IP) at 250 μg 3 times a week. For the combination treatment, ABI-009 is administered, for example, concurrently with, 1 week prior to, or 1 week following the administration of anti-PD-1 antibody. The animals in each group are monitored, for example, for tumor volume, adverse response, histopathology of tumor, body weight and general health condition (eating, walking, daily activities).

### Example 4: Treatment of Hematological Malignancies with the Combination of nab-Sirolimus and Cancer Vaccines

Immunocompetent mice bearing syngeneic tumors are treated with the combination of ABI-009 and a cancer vaccine. A hematological malignancy cell line, such as human multiple myeloma cell line NCI-H929, is transduced with a tumor-associated antigen, such as the human gp100 gene, to generate, for example, the NCI-H929-gp100 cell line, which is cultured, for example, in RPMI-1640 medium supplemented with 10% FBS, 2 mmol/L glutamine, and 1% penicillin-streptomycin at 37° C. with 5% CO₂. On Day 0, for example, mice, such as female CB.17 SCID mice, are inoculated, for example, subcutaneously with at least 1×10⁶ NCI-H929-gp100 cells (such as subcutaneously with 1×10⁷ NCI-H929 cells).

The cancer vaccine contains, for example, recombinant tumor-associated antigen, such as protein gp100, with an adjuvant, such as recombinant heat shock protein (HSP; hsp110). The adjuvant-based vaccine, such as HSP-based anti-tumor gp100 vaccine, is generated, for example, by incubating and non-covalently complexing gp100 and hsp110 recombinant proteins at an equal molar ratio.

Treatment starts, for example, on Day 10. Mice are divided, for example, into at least one experimental group treated, for example, on Day 10 and Day 17 with the combination of ABI-009 and cancer vaccine, such as gp100 cancer vaccine, and one control group that receives no treatment or mock treatment. ABI-009 is administered, for example, IV at 5 mg/kg. The cancer vaccine, such as gp100 vaccine, is administered, for example, intradermally at 25 μg. The animals in each group are monitored, for example, for tumor volume, adverse response, histopathology of tumor, body weight and general health condition (eating, walking, daily activities).

1. A method of treating a hematological malignancy in an individual, comprising administering to the individual: a) an effective amount of a composition comprising nanoparticles comprising an mTOR inhibitor and an albumin, and b) an effective amount of a second therapeutic agent, wherein the second therapeutic agent is selected from the group consisting of an immunomodulator, a histone deacetylase inhibitor, a kinase inhibitor, and a cancer vaccine.

2. The method of claim 1, wherein the hematological malignancy is multiple myeloma, mantle cell lymphoma, T cell lymphoma, chronic myeloid leukemia, or acute myeloid leukemia.

3. The method of claim 1, wherein the hematological malignancy is relapsed or refractory to a standard therapy for the hematological malignancy.

4. The method of claim 1, wherein the amount of the mTOR inhibitor in the mTOR inhibitor nanoparticle composition is from about 10 mg/m² to about 150 mg/m².

5-6. (canceled)

7. The method of claim 1, wherein the mTOR inhibitor nanoparticle composition is administered weekly or 3 out of every 4 weeks.

8. (canceled)

9. The method of claim 1, wherein the mTOR inhibitor nanoparticle composition and the second therapeutic agent are administered sequentially or simultaneously to the individual.

10. (canceled)

11. The method of claim 1, wherein the mTOR inhibitor is a limus drug.

12. The method of claim 11, wherein the limus drug is sirolimus.

13. The method of claim 1, wherein the average diameter of the nanoparticles in the composition is no greater than about 150 nm.

14. (canceled)

15. The method of claim 1, wherein the weight ratio of the albumin to the mTOR inhibitor in the nanoparticle composition is no greater than about 9:1.

16. The method of claim 1, wherein the nanoparticles comprise the mTOR inhibitor associated with the albumin.

17. (canceled)

18. The method of claim 1, wherein the mTOR inhibitor nanoparticle composition is administered intravenously, intraarterially, intraperitoneally, intravesicularly, subcutaneously, intrathecally, intrapulmonarily, intramuscularly, intratracheally, intraocularly, transdermally, orally, or by inhalation.

19. (canceled)

20. The method of claim 1, wherein the individual is human.

21. The method of claim 1, further comprising selecting the individual for treatment based on the presence of at least one mTOR-activating aberration.

22. The method of claim 21, wherein the mTOR-activating aberration comprises a mutation in an mTOR-associated gene.

23. The method of claim 21, wherein the mTOR-activating aberration is in at least one mTOR-associated gene selected from the group consisting of AKT1, FLT-3, MTOR, PIK3CA, TSC1, TSC2, RHEB, STK11, NF1, NF2, KRAS, NRAS and PTEN.

24. The method of claim 1, wherein the second therapeutic agent is an immunomodulator.

25-31. (canceled)

32. The method of claim 1, wherein the second therapeutic agent is a histone deacetylase inhibitor.

33-36. (canceled)

37. The method of claim 1, wherein the second therapeutic agent is a kinase inhibitor.

38-41. (canceled)

42. The method of claim 1, wherein the second therapeutic agent is a cancer vaccine.

43-46. (canceled)
